SEQUENCE LISTING (32 sequences)

SEQ ID NO: 1 - protein, 9 aa, artificial sequence
  protease site
LKTPRVVGG

SEQ ID NO: 2 - protein, 9 aa, artificial sequence
  protease cleavage site
TRQARVVGG

SEQ ID NO: 3 - protein, 9 aa, artificial sequence
  protease cleavage site
DDDDKIVGG

SEQ ID NO: 4 - protein, 6 aa, artificial sequence
  protease cleavage site
DDDDKI

SEQ ID NO: 5 - DNA, 7033 bp, artificial sequence
  plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatcgccaccatggagacagacacactcctgctatgggtactgctgctctgggttccaggttccactggtgacgctccggacaggagtgaccaggagccgctgtacccagtgcaggtcagctctgcggacgctcggctcatggtctttgacaagacggaagggacgtggcggctgctgtgctcctcgcgctctaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggacgacgacgacaagatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgatttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctcgaattcggtaagcctatccctaaccctctcctcggtctcgattctacgcgtaccggtcatcatcaccatcaccattgagtttaaagcggccgcatagataactgatccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagactcgagatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcgatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 12242 bp, artificial sequence
  plasmid 2
tcgagcggccgctttaaactcaatggtgatggtgatgatgaccggtacgcgtagaatcgagaccgaggagagggttagggataggcttaccgaattcgagctgggtcaccatgccgctggcttcggagtgagtctttatggcctggaagatccactcccggaagtcactgactttggtgtagacgcctggcttctgggccagggcacagccagtgccccaactcacaatgccacacagccgccaacgtggcgtccgagagatgctgtcctcacacacaaagggaccaccgctgtcgccctggcaggcatcaatgccaccctcggggtagccagcacagaacatcttgggcttgatctggtttccatagaaatcagcgccattgcagacatcattgctgattatggggactcgagcctcctggagtaccccggcctgttggccatagtactgcgtgttgccccagcccgtcacggtacagatcttgccatccaccagggcctggccggcagctgggaggcacacaggctggatgtattctgtgaggggcaggggactggagaggtggaccagggcaatatcgttgctgttctcctcgctgttggggtcccgaaagggaagatagcccccgtggtagaccacagcctgcacccccagctgcagaccgtggggagaggcctgggccacggcaccggcaaacactcgccatcgggacaggacccggttccgctccgggaagcagtgggcggctgtcagcacccagtccccggagagcagggatcccccacagaggtgtgctccatcatagcgaaggctgacttgccacggccaccggcccaagctggtgtcccggcctcccacgatcttgtcgtcgtcgtccacgggcagcttcctgcggccacagtcttggcagatggcggccaagaaacggcctctggggcaatcacacacggagatgacctccagcagcctctgggtgtggggcagcctcccctcgtccacacagaagaagcccgacgtgccattggcgcccgccgttcgcacgtccagctcggagtgggtcagtgccctgaggaagcccatctcctcgcagctgagtccggctaccctggcgttagagcgcgaggagcacagcagccgccacgtcccttccgtcttgtcaaagaccatgagccgagcgtccgcagagctgacctgcactgggtacagcggctcctggtcactcctatccggagcgtcaccagtggaacctggaacccagagcagcagtacccatagcaggagtgtgtctgtctccatggtggcgatctggtacccagcttctagagatctgacggttcactaaacgagctctgcttatatagacctcccaccgtacacgcctaccgcccatttgcgtcaacggggcggggttattacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtcatattggacatgagccaatataaatgtacatattatgatatagatacaacgtatgcaatggccaatagccaatattgatttatgctatataaccaatgactaatatggctaattgccaatattgattcaatgtatagatcttccatacctaccagttctgcgcctgcagcaatgcaacaacgttgcccggatctgcgatgataagctgtcaaacatgagaattggtcgactagcttggcacgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggaccagaccccacgcaacgcccaaaataataacccccacgaaccataaaccattccccatgggggaccccgtccctaacccacggggccagtggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccgggatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcatgcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgccgtagtcaggtttagttcgtccggcggcggggctctagagtcgaccggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagccggatcataatcagccataccacatttgtagaggttttacttgctttaaaaaacctccccacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccacgtgcaggcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattc

SEQ ID NO: 7 - DNA, 11924 bp, artificial sequence
  plasmid 3
tcgagcggccgctttaaactcaatggtgatggtgatgatgaccggtacgcgtagaatcgagaccgaggagagggttagggataggcttaccgaattcgagctgggtcaccatgccgctggcttcggagtgagtctttatggcctggaagatccactcccggaagtcactgactttggtgtagacgcctggcttctgggccagggcacagccagtgccccaactcacaatgccacacagccgccaacgtggcgtccgagagatgctgtcctcacacacaaagggaccaccgctgtcgccctggcaggcatcaatgccaccctcggggtagccagcacagaacatcttgggcttgatctggtttccatagaaatcagcgccattgcagacatcattgctgattatggggactcgagcctcctggagtaccccggcctgttggccatagtactgcgtgttgccccagcccgtcacggtacagatcttgccatccaccagggcctggccggcagctgggaggcacacaggctggatgtattctgtgaggggcaggggactggagaggtggaccagggcaatatcgttgctgttctcctcgctgttggggtcccgaaagggaagatagcccccgtggtagaccacagcctgcacccccagctgcagaccgtggggagaggcctgggccacggcaccggcaaacactcgccatcgggacaggacccggttccgctccgggaagcagtgggcggctgtcagcacccagtccccggagagcagggatcccccacagaggtgtgctccatcatagcgaaggctgacttgccacggccaccggcccaagctggtgtcccggcctcccacgatcttgtcgtcgtcgtccacgggcagcttcctgcggccacagtcttggtccggagcgtcaccagtggaacctggaacccagagcagcagtacccatagcaggagtgtgtctgtctccatggtggcgatctggtacccagcttctagagatctgacggttcactaaacgagctctgcttatatagacctcccaccgtacacgcctaccgcccatttgcgtcaacggggcggggttattacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtcatattggacatgagccaatataaatgtacatattatgatatagatacaacgtatgcaatggccaatagccaatattgatttatgctatataaccaatgactaatatggctaattgccaatattgattcaatgtatagatcttccatacctaccagttctgcgcctgcagcaatgcaacaacgttgcccggatctgcgatgataagctgtcaaacatgagaattggtcgactagcttggcacgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggaccagaccccacgcaacgcccaaaataataacccccacgaaccataaaccattccccatgggggaccccgtccctaacccacggggccagtggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccgggatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcatgcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgccgtagtcaggtttagttcgtccggcggcggggctctagagtcgaccggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagccggatcataatcagccataccacatttgtagaggttttacttgctttaaaaaacctccccacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccacgtgcaggcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattc

SEQ ID NO: 8 - protein, 417 aa, Homo sapiens
MAQKEGGRTVPCCSRPKVAALTAGTLLLLTAIGAASWAIVAVLLRSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDRIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQL

SEQ ID NO: 9 - protein, 425 aa, Homo sapiens
METDTLLLWVLLLWVPGSTGDAAARSDQEPLYPVQVSSADARLMVFDKTEGTWRLLCSSRSNARVAGLSCEEMGFLRALTHSELDVRTAGANGTSGFFCVDEGRLPHTQRLLEVISVCDCPRGRFLAAICQDCGRRKLPVDDDDKIVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQLEFGKPIPNPLLGLDSTRTGHHHHHH

SEQ ID NO: 10 - DNA, 30 bp, artificial sequence
  primer
agaggcagtgacatggcgcagaaggagggt

SEQ ID NO: 11 - DNA, 21 bp, artificial sequence
  primer
tggaggctgcgcagcgagaag

SEQ ID NO: 12 - DNA, 21 bp, artificial sequence
  primer
tcgagtccccataatcagcaa

SEQ ID NO: 13 - DNA, 22 bp, artificial sequence
  primer
catcttgggcttgatctggttt

SEQ ID NO: 14 - DNA, 28 bp, artificial sequence
  probe
atgtctgcaatggcgctgacttctatgg

SEQ ID NO: 15 - DNA, 22 bp, artificial sequence
  primer
aggtcatctccgtgtgtgattg

SEQ ID NO: 16 - DNA, 16 bp, artificial sequence
  primer
cccacgatgcggtcca

SEQ ID NO: 17 - DNA, 21 bp, artificial sequence
  probe
cagaggccgtttcttggccgc

SEQ ID NO: 18 - DNA, 30 bp, artificial sequence; identical to SEQ ID NO: 10
  primer

SEQ ID NO: 19 - DNA, 21 bp, artificial sequence
  primer
tggaggctgcgcagcgagaag

SEQ ID NO: 20 - DNA, 27 bp, artificial sequence
  primer
cagctcgaattcggtaagcctatccct

SEQ ID NO: 21 - DNA, 27 bp, artificial sequence
  primer
gatgcggccgctttaaactcaatggtg

SEQ ID NO: 22 - DNA, 27 bp, artificial sequence
  primer
catatgcccgggaggagtgaccaggag

SEQ ID NO: 23 - DNA, 27 bp, artificial sequence
  primer
cttaccgaattcgagctgggtcaccat

SEQ ID NO: 24 - DNA, 30 bp, artificial sequence
  primer
ctgatccggacaggagtgaccaggagccgc

SEQ ID NO: 25 - DNA, 19 bp, artificial sequence
  primer
gccgggtcccaggaaagga

SEQ ID NO: 26 - DNA, 66 bp, artificial sequence
  primer
gatcgatatcgccaccatggagacagacacactcctgctatgggtactgctgctctgggttccagg

SEQ ID NO: 27 - DNA, 46 bp, artificial sequence
  primer
atcgtccggagcgtcaccagtggaacctggaacccagagcagcagt

SEQ ID NO: 28 - DNA, 35 bp, Artificial Sequence
  primer
gagatccggaccaagactgtggccgtaggaagctg

SEQ ID NO: 29 - DNA, 19 bp, artificial sequence
  primer
gccgggtcccaggaaagga

SEQ ID NO: 30 - DNA, 32 bp, artificial sequence
  primer
tgcaggtacctaggagtgaccaggagccgctg

SEQ ID NO: 31 - DNA, 33 bp, artificial sequence
  primer
ccggggtaccagctgggtcaccatgccgctggc

SEQ ID NO: 32 - protein, 5 aa, Homo sapiens
RIVGG

(19) United States
(12) Patent Application Publication
(10) Pub. No.: US 2004/0132156 A1
(43) Pub. Date: Jul. 8, 2004

(54) Modified hepsin molecules having a substitute activation sequence and uses thereof

(75) Inventors: Gordon Parry, Oakland, CA (US); David Vogel, Richmond, CA (US); Marc Whitlow, Richmond, CA (US); Qingyu Wu, Lafayette, CA (US)

Correspondence Address: Wendy Washtien,, Berlex Biosciences, Patent Department, 2600 Hilltop Drive Avenue, P.O. Box 4099, Richmond, CA, 94804-0099, US

(73) Assignee: Schering Aktiengesellschaft, Berlin (DE)

(21) Appl. No.: 10/678,816

(22) Filed: Oct. 2, 2003

Related U.S. Application Data
(60) Provisional application No. 60/416,038, filed on Oct. 4, 2002.

(51) Int. Cl. C12N 9/64; C07H 21/04

(52) U.S. Cl. 435/226; 435/69.1; 435/320.1; 435/325; 536/23.2

(57) ABSTRACT

The present invention provides modified hepsin molecules, or fragments or derivatives thereof, including those having a substitute activation sequence. The modified hepsin molecules are cleaved at the substitute activation sequence, thereby generating activated modified hepsin molecule, or fragments or derivatives thereof, that exhibit the functional activity of naturally-occurring, wild-type hepsin molecules.

This application claims priority to U.S. Provisional Application Serial No. 60/416,038 filed Oct. 4, 2002, the entirety of which is incorporated herein by reference.

Throughout this application various publications are referenced. The disclosures of these publications are hereby incorporated by reference in their entirety into this application in order to more fully describe the state of the art to which the invention pertains.

### FIELD OF INVENTION

The present invention relates to modified hepsin molecules, or fragments or derivatives thereof, including those having a substitute activation sequence.

### BACKGROUND

Hepsin is a transmembrane serine protease that was originally identified from a human hepatoma HepG2 cell library using a homology-based cloning strategy (Leytus, S. P., K. R. Loeb, F. S. Hagen, K. Kurachi, and E. W. Davie. 1988. A novel trypsin-like serine protease (hepsin) with a putative transmembrane domain expressed by human liver and hepatoma cells. Biochemistry, 27 (3):1067-74). Like most of the trypsin-like serine proteases, wildtype hepsin is synthesized as a zymogen. Hepsin cDNA encodes a polypeptide of 417 amino acids. At its amino terminus, hepsin includes a cytoplasmic domain and an integral transmembrane domain. In the extracellular region of hepsin, there is a macrophage scavenger receptor-like domain and a trypsin-like protease domain at the carboxyl terminus. The overall topology of hepsin is similar to those of other type II transmembrane serine proteases of the trypsin superfamily (Hooper, J. D., J. A. Clements, J. P. Quigley, and T. M. Antalis. 2001. Type II transmembrane serine proteases. Insights into an emerging class of cell surface proteolytic enzymes. J Biol. Chem. 276 (2):857-60), which include corin (Yan, W., N. Sheng, M. Seto, J. Morser, and Q. Wu. 1999. Corin, a mosaic transmembrane serine protease encoded by a novel cDNA from human heart. J Biol. Chem. 274 (21):14926-35; Yan, W., F. Wu, J. Morser, and Q. Wu. 2000. Corin, a transmembrane cardiac serine protease, acts as a pro-atrial natriuretic peptide-converting enzyme. Proc Natl Acad Sci U.S.A. 97 (15):8525-9), enterokinase (Kitamoto, Y., X. Yuan, Q. Wu, D. W. McCourt, and J. E. Sadler. 1994. Enterokinase, the initiator of intestinal digestion, is a mosaic protease composed of a distinctive assortment of domains. Proc Natl Acad Sci USA. 91 (16):7588-92), MT-SP1/matryptase (Takeuchi, T., M. A. Shuman, and C. S. Craik. 1999. Reverse biochemistry: use of macromolecular protease inhibitors to dissect complex biological processes and identify a membrane-type serine protease in epithelial cancer and normal tissue. Proc Natl Acad Sci USA. 96 (20):11054-61; Lin, C. Y., J. Anders, M. Johnson, Q. A. Sang, and R. B. Dickson. 1999. Molecular cloning of cDNA for matriptase, a matrix-degrading serine protease with trypsin-like activity. J Biol. Chem. 274 (26):18231-6), human airway trypsin-like protease (Yamaoka, K., K. Masuda, H. Ogawa, K. Takagi, N. Umemoto, and S. Yasuoka. 1998. Cloning and characterization of the cDNA for human airway trypsin-like protease. J Biol. Chem. 273 (19):11895-901), TMPRSS2 (Paoloni-Giacobino, A., H. Chen, M. C. Peitsch, C. Rossier, and S. E. Antonarakis. 1997. Cloning of the TMPRSS2 gene, which encodes a novel serine protease with transmembrane, LDLRA, and SRCR domains and maps to 21q22.3. Genomics. 44 (3):309-20) and Stubble-stubbloid (Appel, L. F., M. Prout, R. Abu-Shumays, A. Hammonds, J. C. Garbe, D. Fristrom, and J. Fristrom. 1993. The Drosophila Stubble-stubbloid gene encodes an apparent transmembrane serine protease required for epithelial morphogenesis. Proc Natl Acad Sci USA. 90 (11):4937-41).

Biochemical studies indicate that activated hepsin is an enzyme with trypsin-like substrate specificity that cleaves peptide bonds after basic residues such as arginine and lysine (Kurachi, K., A. Torres-Rosado, and A. Tsuji. 1994. Hepsin. Methods Enzymol. 244:100-14; Wu, Q. 2001. Gene targeting in hemostasis. Hepsin. Front Biosci. 6:D192-200.). Expression and characterization of recombinant hepsin show that the protein is synthesized as a single-chain molecule with an apparent molecular mass of ˜51 kDa. Tryptic digestion experiments confirmed hepsin is a type II transmembrane protein (Kazama, Y., T. Hamamoto, D. C. Foster, and W. Kisiel. 1995. Hepsin, a putative membrane-associated serine protease, activates human factor VII and initiates a pathway of blood coagulation on the cell surface leading to thrombin formation. J Biol. Chem. 270 (1):66-72; Tsuji, A., A. Torres-Rosado, T. Arai, M. M. Le Beau, R. S. Lemons, S. H. Chou, and K. Kurachi. 1991. Hepsin, a cell membrane-associated protease. Characterization, tissue distribution, and gene localization. J Biol. Chem. 266 (25):16948-53).

To date, the physiological function of hepsin has not been elucidated. In vitro experiments suggest that hepsin may play a role in blood coagulation (Kazama, Y., T. Hamamoto, D. C. Foster, and W. Kisiel. 1995. Hepsin, a putative membrane-associated serine protease, activates human factor VII and initiates a pathway of blood coagulation on the cell surface leading to thrombin formation. J Biol. Chem. 270 (1):66-72), hepatocyte growth (Torres-Rosado, A., O. S. KS, A. Tsuji, S. H. Chou, and K. Kurachi. 1993. Hepsin, a putative cell-surface serine protease, is required for mammalian cell growth. Proc Natl Acad Sci USA. 90 (15):7181-5) and fertilization (Vu, T. K. H., R. W. Liu, C. J. Haaksma, J. J. Tomasek, and E. W. Howard. 1997. Identification and cloning of the membrane-associated serine protease, hepsin, from mouse preimplantation embryos. J Biol. Chem. 272 (50):31315-20). Two independent knock-out experiments, however, showed that hepsin-deficient mice were viable, fertile and grew normally (Wu, Q., D. Yu, J. Post, M. Halks-Miller, J. E. Sadler, and J. Morser. 1998. Generation and characterization of mice deficient in hepsin, a hepatic transmembrane serine protease. J Clin Invest. 101 (2):321-6; Yu, I. S., H. J. Chen, Y. S. Lee, P. H. Huang, S. R. Lin, T. W. Tsai, and S. W. Lin. 2000. Mice deficient in hepsin, a serine protease, exhibit normal embryogenesis and unchanged hepatocyte regeneration. Thromb Haemost. 84:865-70). No apparent defects in hemostasis or liver function were identified in hepsin-deficient mice. These results indicate that hepsin is not essential for either embryonic development or maintenance of normal liver and hemostatic functions in mice.

Hepsin is expressed in several human tissues including liver, kidney and prostate (Kurachi, K., A. Torres-Rosado, and A. Tsuji. 1994. Hepsin. Methods Enzymol. 244:100-14; Wu, Q. 2001. Gene targeting in hemostasis. Hepsin. Front Biosci. 6:D192-200). Hepsin mRNA is most abundantly expressed in the liver. Low levels of hepsin mRNA expression were also detected in other tissues including kidney, thyroid, pancreas, testis and prostate.

In addition to its expression in normal tissues, hepsin mRNA expression was also reported in several types of cancer such as hepatoma, ovarian cancer and kidney carcinoma (Torres-Rosado, A., O'Shea, KS, A., Tsuji, S. H. Shou, and K. Kurachi. 1993. Hepsin, a putative cell-surface serine protease, is required for mammalian cell growth. Proc Natl Acad Sci USA. 90 (15):7181-5; Leytus, S. P., K. R. Loeb, F. S. Hagen, K. Kurachi, and E. W. Davie. 1988. A novel trypsin-like serine protease (hepsin) with a putative transmembrane domain expressed by human liver and hepatoma cells. Biochemistry. 27 (3):1067-74; Tanimoto, H., Y. Yan, J. Clarke, S. Korourian, K. Shigemasa, T. H. Parmley, G. P. Parham, and O. B. T J. 1997. Hepsin, a cell surface serine protease identified in hepatoma cells, is overexpressed in ovarian cancer. Cancer Res. 57 (14):2884-7; Zacharski, L. R., D. L. Ornstein, V. A. Memoli, S. M. Rousseau, and W. Kisiel. 1998. Expression of the factor VII activating protease, hepsin, in situ in renal cell carcinoma [letter]. Thromb Haemost. 79 (4):876-7.)

Recently, several independent investigators found that hepsin mRNA was highly upregulated in advanced prostate cancer compared to that in normal prostate tissues or benign prostatic hyperplasia (BPH) (Luo, J., D. J. Duggan, Y. Chen, J. Sauvageot, C. M. Ewing, M. L. Bittner, J. M. Trent, and W. B. Isaacs. 2001. Human prostate cancer and benign prostatic hyperplasia: molecular dissection by gene expression profiling. Cancer Res. 61 (12):4683-8; Magee, J. A., T. Araki, S. Patil, T. Ehrig, L. True, P. A. Humphrey, W. J. Catalona, M. A. Watson, and J. Milbrandt. 2001. Expression profiling reveals hepsin overexpression in prostate cancer. Cancer Res. 61:5692-5696; Welsh, J. B., L. M. Sapinoso, A. I. Su, S. G. Kern, J. Wang-Rodriguez, C. A. Moskaluk, H. F. Frierson, and G. M. Hampton. 2001. Analysis of gene expression identifies candidate markers and pharmacological targets in prostate cancer. Cancer Res. 61:5974-5978; Dhanasekaran, S. M., T. R. Barrette, D. Ghosh, R. Shah, S. Varambally, K. Kurachi, K. J. Pienta, M. A. Rubin, and A. M. Chinnaiyan. 2001. Delineation of prognostic biomarkers in prostate cancer. Nature. 412 (6849):822-6; Stamey, T. A., J. A. Warrington, M. C. Caldwell, Z. Chen, Z. Fan, M. Mahadevappa, J. E. McNeal, R. Nolley and Z. Zhang. 2001. Molecular genetic profiling of Gleason grade 4/5 prostate cancers compared to benign prostatic hyperplasia. J Urol. 166(6):2171-7; Ernst, T., M. Hergenhahn, M. Kenzelmann, C. D. Cohen, M. Bonrouhi, A. Weninger, R. Klaren, E. F. Grone, M. Wiesel, C. Gudemann, J. Kuster, W. Schott, G. Staehler, M. Kretzler, M. Hollstein and H. J. Grone. 2002. Decrease and gain of gene expression are equally discriminatory markers for prostate carcinoma: a gene expression analysis on total and microdissected prostate tissue. Am J Pathol. 160(6):2169-80). In these studies, using Affymetrix chips that covered over 9,900 genes, hepsin mRNA expression was found to be 30- to 42-fold higher in advanced prostate cancer tissues than in normal prostate tissues or BPH. The overexpression of hepsin mRNA appeared to correlate inversely with measures of patient prognosis.

The finding of hepsin mRNA overexpression in cancers suggests that hepsin may contribute to tumor-related angiogenesis or cancer invasion and metastasis. Serine proteases are known to have growth factor-like activities. Thrombin, for example, is a potent mitogen for vascular fibroblasts and smooth muscle cells (Fenton, J. W. d. 1986. Thrombin. Ann NY Acad. Sci. 485:5-15). Furthermore, several growth factors, such as hepatocyte growth factor (HGF) and the product of growth arrest-specific gene 6 (Gas6), share striking sequence and structural similarities with blood coagulation proteases (Nakamura, T., T. Nishizawa, M. Hagiya, T. Seki, M. Shimonishi, A. Sugimura, K. Tashiro, and S. Shimizu. 1989. Molecular cloning and expression of human hepatocyte growth factor. Nature, 342 (6248):440-3; Manfioletti, G., C. Brancolini, G. Avanzi, and C. Schneider. 1993. The protein encoded by a growth arrest-specific gene (gas6) is a new member of the vitamin K-dependent proteins related to protein S, a negative coregulator in the blood coagulation cascade. Mol Cell Biol. 13 (8):4976-85). HGF is critical for the development of several epithelial organs including liver and placenta (Schmidt, C., F. Bladt, S. Goedecke, V. Brinkmann, W. Zschiesche, M. Sharpe, E. Gherardi, and C. Birchmeier. 1995. Scatter factor/hepatocyte growth factor is essential for liver development. Nature. 373 (6516):699-702; Uehara, Y., O. Minowa, C. Mori, K. Shiota, J. Kuno, T. Noda, and N. Kitamura. 1995. Placental defect and embryonic lethality in mice lacking hepatocyte growth factor/scatter factor. Nature. 373 (6516):702-5).

As a serine protease, hepsin may act as a growth factor to stimulate proliferation of cancer cells. In fact, hepsin was reported to be essential for hepatocyte growth in culture (Torres-Rosado, A., O. S. KS, A. Tsuji, S. H. Chou, and K. Kurachi. 1993. Hepsin, a putative cell-surface serine protease, is required for mammalian cell growth. Proc Natl Acad Sci USA. 90 (15):7181-5). Alternatively, hepsin may act as a processing enzyme for the activation of polypeptide growth hormones. Our recent discovery of corin as a pro-atrial natriuretic peptide convertase suggests that type II transmembrane serine proteases are important in the activation of peptide hormones (Yan, W., N. Sheng, M. Seto, J. Morser, and Q. Wu. 1999. Corin, a mosaic transmembrane serine protease encoded by a novel cDNA from human heart. J Biol. Chem. 274 (21):14926-35; Yan, W., F. Wu, J. Morser, and Q. Wu. 2000. Corin, a transmembrane cardiac serine protease, acts as a pro-atrial natriuretic peptide-converting enzyme. Proc Natl Acad Sci U.S.A. 97 (15):8525-9). In addition, hepsin may also contribute to the degradation of extracellular matrix proteins, either directly, by its proteolytic activity, or indirectly, by activating other matrix metalloproteinases. Proteolytic digestion of extracellular matrix proteins is a critical step in tumor progression and metastasis (Mignatti, P., and D. B. Rifkin. 1993. Biology and biochemistry of proteinases in tumor invasion. Physiol Rev. 73 (1):161-95).

If hepsin is indeed critical in cancer progression, it represents an excellent target for drug development for the following reasons. First, hepsin is a trypsin-like serine protease. Trypsin-like serine proteases are pharmaceutical drug targets (Drews, J., 2000, Drug Discovery: a historical perspective. Science 287:1960-4). Second, hepsin is a cell surface protein that should be readily accessible to therapeutic agents in the circulation. Third, inhibition of hepsin is expected to have minimal side effects, because hepsin-deficient mice are viable, fertile and grow normally.

Naturally-occurring i.e. wildtype, hepsin molecules include an activation sequence which is recognized and cleaved by an active trypsin-like protease, which has not yet been identified, and by other type II transmembrane serine proteases. Therefore, the zymogen form of the naturally-occurring hepsin molecules are activated by other proteases. Additionally, once activated, the activated form of the naturally-occurring hepsin molecules are short-lived, making it difficult to produce anti-hepsin antibodies. Thus, there is a need for stable hepsin molecules.

### SUMMARY

The present invention provides modified hepsin molecules, or fragments or derivatives thereof, comprising a substituted activation sequence differing from the wildtype hepsin activation sequence, and methods to encode, express and use stable modified hepsin molecules.

In one embodiment, the modified hepsin molecule is a modified hepsin zymogen, or fragments or derivatives thereof, comprising a substituted activation sequence differing from the wildtype hepsin activation sequence.

Additionally, in another embodiment, the modified hepsin molecule is an activated, modified hepsin, or fragments or derivatives thereof, wherein a substituted activation sequence, differing from the wildtype hepsin activation sequence, is cleaved, rendering the hepsin active.

The present invention provides fusion molecules comprising a modified hepsin molecule, or fragments or derivatives thereof, fused to a non-hepsin molecule. In one embodiment, the non-hepsin molecule is an epitope tag or a reporter molecule. The invention further provides methods to encode, express and use modified hepsin fusion molecules.

The present invention provides chimeric molecules comprising a portion of a hepsin molecule, isolated from a first source, fused to a portion of a hepsin molecule, isolated from a second, different source. The invention further provides methods to encode, express and use chimeric hepsin molecules.

The present invention also provides anti-hepsin antibodies, or fragments or derivatives thereof. The antibodies can be polyclonal, monoclonal, chimeric, humanized, human, internalizing, neuralizing, anti-idiotypic antibodies, recombinant proteins having immunologically-activity, or immunoconjugates which bind a target polypeptide. The invention further provides methods to generate and use anti-hepsin antibodies.

The present invention provides a host-vector system comprising a vector comprising a nucleotide sequence encoding a modified hepsin molecule, or a fragment or derivative thereof, introduced into a suitable host cell. The invention further provides methods to make and use the host-vector system.

The present invention provides a pharmaceutical composition comprising a pharmaceutically acceptable carrier and a composition of the invention. In one embodiment the pharmaceutical composition comprises a pharmaceutically acceptable carrier admixed with a hepsin molecule of the invention. In another embodiment, the pharmaceutical composition comprises a pharmaceutically acceptable carrier admixed with an anti-hepsin antibody.

The present invention provides assays to: 1) identify molecules that interact with, compete with and/or inhibit the compositions of the invention, 2) characterize the compositions of the invention, 3) localize hepsin expression in a cell and/or tissue, 4) detect the presence of hepsin in a cell and/or tissue sample; and 5) quantitate the amount of hepsin in a cell and/or tissue sample.

Kits comprising compositions, as described infra, are also encompassed by the invention. In one embodiment, a kit comprising one or more of the compositions of the invention is used in a screening assay to identify hepsin inhibitors. In another embodiment, a kit comprising one or more of the compositions of the invention is used in a screening assay to identify activated hepsin molecules.

Methods for using the compositions of the invention are provided. The hepsin compositions can be used to identify and isolate molecules that interact, inhibit and/or compete with hepsin. The compositions of the invention can be used to localize and/or characterize hepsin. The compositions can also be used to treat diseases associated with over-, under- and/or aberrant-expression of hepsin.

Methods for using the antibodies of the invention include: methods for treating a hepsin associated disease such as cancer, where the antibodies inhibit the growth of, or kill, cancer cells expressing or over-expressing hepsin; methods for purifying hepsin; methods to isolate and/or enrich for cells expressing hepsin; and immunohistochemical staining methods.

### BRIEF DESCRIPTION OF FIGURES

FIG. 1: A Northern blot analysis of hepsin mRNA levels in normal human tissues, as described in Example 1, infra.

FIG. 2: A graph showing the results of Taqman PCR analysis of hepsin mRNA levels in normal human tissues, as described in Example 1, infra.

FIG. 3: Hepsin mRNA levels in prostate cancer; a comparison of hepsin mRNA levels in normal and prostate cancer, as described in Example 1, infra. A) A Northern blot analysis of hepsin mRNA levels in normal, benign prostate hyperplasia, primary prostate cancer, and advanced prostate cancer samples. B) A graph showing the fold-increase of hepsin mRNA levels in normal, benign prostate hyperplasia, primary prostate cancer, and advanced prostate cancer samples.

FIG. 4: A graph showing the results of Taqman PCR analysis of hepsin mRNA levels in a prostate cancer cell line (LNCaP), prostate benign hyperplasia, and advanced prostate cancer samples, as described in Example 1, infra.

FIG. 5: Northern analysis of hepsin mRNA levels in prostate-derived cell lines. A) A Northern blot analysis of hepsin mRNA levels in various prostate-derived cell lines, as described in Example 1, infra. B) A photograph of the agarose gel used to generate the Northern blot, shown in FIG. 5A, stained with ethidium bromide to show equal sample loading.

FIG. 6: A graph showing the results of Taqman PCR analysis of hepsin mRNA levels in various prostate-derived cell lines, as described in Example 1, infra.

FIG. 7: Up-regulation of hepsin mRNA levels in LNCaP cells by dihydrotestosterone (DHT). A) A Northern blot analysis of hepsin mRNA levels in LNCaP cells treated with dihydrotestosterone, as described in Example 1, infra. B) A graph showing the fold-increase in hepsin mRNA levels in cells treated with dihydrotestosterone, as described in Example 1, infra.

FIG. 8: A schematic representation of human hepsin molecule and modified hepsin molecules. Top) Human hepsin molecule, including the cytoplasmic, transmembrane, and extracellular domains. A) Modified hepsin molecule, including a signal peptide sequence, the extracellular domain, and epitope tags. B) Modified hepsin molecule, including a signal peptide sequence, the extracellular domain having a substitute activation sequence, and epitope tags.

FIG. 9: The nucleotide sequence of a plasmid pIRESpuro2W/hepEK (SEQ ID NO: 5) encoding the extracellular domain of a modified human hepsin molecule with an enterokinase cleavage site, as described in Example 2, infra. Underlying brackets delineate codons (three nucleotides), each encoding an amino acid. Incomplete codons wrap around to the start of the next line. The amino acid represented by the incomplete codon is designated by a dot next to it so that the same amino acid is identified a second time at the start of the next line. Those skilled in the art will recognize that an amino acid is encoded by three nucleotides and accordingly realize that the amino acid encoded by the wrap-around codon is named twice.

FIG. 10: The nucleotide sequence of a plasmid pCEP4W/hepEK (SEQ ID NO: 6) encoding the extracellular domain of a modified human hepsin molecule with an enterokinase cleavage site, as described in Example 2, infra. Like FIG. 9, incomplete codons wrap around to the start of the next line. Therefore, the amino acid represented by the incomplete codon is designated by a dot next to it so that the same amino acid is identified a second time at the start of the next line. Accordingly, the amino acid encoded by the wrap-around codon is named twice.

FIG. 11: The nucleotide sequence of a plasmid pCEP4W/hep36, which is also known as pCEP4W/hepEK36 (SEQ ID NO: 7), encoding a fragment of the extracellular domain of a modified human hepsin molecule with an enterokinase cleavage site, as described in Example 2, infra. Like FIG. 9, incomplete codons wrap around to the start of the next line. Therefore, the amino acid represented by the incomplete codon is designated by a dot next to it so that the same amino acid is identified a second time at the start of the next line. Accordingly, the amino acid encoded by the wrap-around codon is named twice.

FIG. 12: A Western blot of modified hepsin molecule expressed in 293EBNA cells, as described in Example 3, infra.

FIG. 13: A Western blot showing an isolated modified hepsin molecule expressed in baculovirus infected insect cells, as described in Example 3, infra.

FIG. 14: A Western blot showing enterokinase (EKMax) processing of hepsinEDEK protein to generate active hepsin enzyme, as described in Example 4, infra.

FIG. 15A: Western Blots showing that monoclonal antibodies 11C1 and 47A5 bind to hepsin as described in Example 6, infra.

FIG. 15B: Western Blots showing that monoclonal antibodies 38E2 and 31C1 bind to hepsin as described in Example 6, infra.

FIG. 15C: Western Blots showing that monoclonal antibodies 46D12, 37G10 and 14C7 bind to hepsin as described in Example 6, infra.

FIG. 15D: Western Blots showing that monoclonal antibodies 72H6 and 14C7 bind to hepsin as described in Example 6, infra.

FIG. 16A: Immunohistochemical staining of human prostate tumor tissue as described in Example 9, infra. Left Panel: Control staining using preimmunized mouse serum from a hepsinknock-out mouse to stain prostate tumor tissue. Right Panel: Staining prostate tumor tissue with anti-Hepsin mouse polyclonal antibody serum generated from a hepsin knock-out mouse immunized with a modified hepsin molecule.

FIG. 16B: Immunohistochemical staining of human prostate tumor tissue as described in Example 9, infra. Left Panel: Staining prostate tumor tissue with culture medium only (control). Right Panel: Staining tissue with culture medium from anti-Hepsin mouse hybridoma 11C1.

FIG. 16C: Immunohistochemical staining of human prostate tumor tissue, as described in Example 9, infra, with anti-Hepsin monoclonal antibody 11C1.

FIG. 17: Amino acid sequence of wildtype Hepsin (SEQ ID NO: 8). The hepsin ectodomain begins with arginine at position 45 and ends with leucine at position 417. The transmembrane domain starts at valine at position 18 and ends at leucine at position 44. The cytoplasmic domain starts at methionine at position 1 and ends at lysine at position 17.

FIG. 18: Amino acid sequence of soluble modified Hepsin (Hep-ED-EK) i.e, the hepsin ectodomain with the enterokinase substitute activation sequence and V5 and 6 His tag (SEQ ID NO: 9). The hepsin ectodomain starts at arginine at position 1 and ends at leucine at position 376. The V5 and 6 His tag sequence starts at glutamic acid at position 377 and ends at histidine at position 401.

FIG. 19: A bar graph demonstrating neutralization of hepsin activity with purified monoclonal antibodies generated as described in Example 5, infra.

### DETAILED DESCRIPTION OF THE INVENTION

Definitions

All scientific and technical terms used in this application have meanings commonly used in the art unless otherwise specified. As used in this application, the following words or phrases have the meanings specified.

As used herein, “hepsin” refers to a transmembrane polypeptide molecule having a cytoplasmic, transmembrane and extracellular domain (also refered to herein as the ectodomain) with an activation site, or fragments or derivatives thereof. The term “hepsin” includes: wildtype hepsin including hepsin zymogens, activated hepsin molecules and fragments or derivatives thereof; and modified hepsin molecules, including modified hepsin zymogens, modified activated hepsin molecules and fragments or derivatives thereof.

As used herein, “wild type” refers to a nucleic acid or polypeptide molecule having the same nucleotide and/or amino acid sequence as a naturally-occurring molecule, respectively. A wildtype hepsin polypeptide molecule has the amino acid sequence of naturally occurring hepsin as shown in FIG. 17 or in Leytus et al., (1988 Biochemistry 27:1067-1074), or any fragment or portion thereof. Wildtype hepsin is synthesized as a zymogen, i.e., an enzyme precursor, which is activated upon cleavage of an activation site in its extracellular domain.

As used herein, the term “activity” refers to a molecule having a function or action. Activity includes enzymatic activity, wherein the molecule is an enzyme e.g., protease, that recognizes, binds, cleaves and/or modifies a substrate.

As used herein, the term “zymogen” refers to a precursor polypeptide molecule having an activation sequence which, upon cleavage by a cognate protease, yields an activated molecule.

An example of a zymogen is a modified hepsin zymogen comprising an enterokinase substitute activation sequence, which becomes activated upon cleavage by enterokinase.

As used herein, the term “activation sequence” refers to an amino acid sequence in a molecule which is cleaved by a cognate protease, and which, when cleaved, renders the molecule biologically active e.g. capable of protease activity. In an activated molecule, the activation sequence is cleaved. An example of an activation sequence in the hepsin molecule is RIVGG.

As used herein, the term “substitute activation sequence” refers to an amino acid sequence that replaces an activation sequence found in a wild type molecule. An example of a substitute activation sequence is DDDDK-IVGG, which is substituted for the naturally occurring activation sequence, RIVGG, in hepsin.

As used herein, the term “modified” refers to molecule with an amino acid or nucleotide sequence differing from a naturally-occurring i.e., wildtype, amino acid or nucleotide, sequence. For example, a modified hepsin molecule can include a substitute activation sequence. A modified molecule can retain the function or activity of a wildtype molecule.

As used herein, the term “derivative” means any modification or alteration of a wildtype molecule. Derivatives include, but are not limited to: a substitution, conservative or non-conservative, in a amino acid and/or nucleotide sequence including substitutions by other amino acids, nucleotides, amino acid analogs or nucleotide analogs; a deletion of one or more amino acids and/or nucleotides; an insertion of one or more amino acids and/or nucleotides; and pre- and/or post-translational modifications. A derivative molecule can share sequence similarity and/or activity with its parent molecule.

As used herein, the term “protease” refers to a class of enzymes which recognizes a molecule and cleaves an activation sequence in the molecule. The protease can be an endopeptidase which cleaves internal peptide bonds. Alternatively, the protease can be an exopeptidase which hydrolyze the peptide bonds from the N-terminal end or the C-terminal end of the polypeptide or protein molecule. The protease folds into a conformation to form a catalytic site which receives and cleaves the activation sequence.

As used herein, the term “catalytic site” refers to a region in a folded protease which receives and cleaves the activation sequence.

As used herein, the term “ligand” refers to any molecule that interacts with hepsin. A ligand can be a molecule that recognizes and binds to hepsin. Alternatively, a ligand can be a molecule recognized and bound by hepsin. For example, a substrate that hepsin binds to and cleaves can be a ligand. In another example, a molecule that binds to and cleaves hepsin can be a ligand.

An anti-hepsin antibody can also be a ligand. As used herein, the term “serine protease” refers to a class of protease enzymes characterized by the presence of a unique serine residue that forms part of the catalytic site in the enzyme. In general, each serine protease member has a different substrate specificity.

As used herein, a first nucleotide or amino acid sequence is said to have sequence “identity” to a second nucleotide or amino acid sequence, respectively, when a comparison of the first and the second sequences shows that they are exactly alike.

As used herein, a first nucleotide or amino acid sequence is said to be “similar” to a second sequence when a comparison of the two sequences shows that they have few sequence differences (i.e., the first and second sequences are nearly identical). For example, two sequences are considered to be similar to each other when the percentage of nucleotides or amino acids that differ between the two sequences can be between about 60% to 99.99%.

As used herein, the term “complementary” refers to nucleic acid molecules having purine and pyrimidine nucleotide bases which have the capacity to associate through hydrogen bonding to form base pairs thereby mediating formation of double stranded nucleic acid molecules. The following base pairs are related by complementarity: guanine and cytosine; adenine and thymine; and adenine and uracil. Complementary applies to all base pairs comprising two single-stranded nucleic acid molecules, or to all base pairs comprising a single-stranded nucleic acid molecule folded upon itself.

As used herein, the term “conservative” refers to substituting an amino acid residue for a different amino acid residue that has similar chemical properties. A conservative amino acid substitution includes: substituting any hydrophobic (e.g., nonpolar) amino acid for any other hydrophobic amino acid; or substituting any hydrophilic (polar, uncharged) amino acid for any other hydrophilic amino acid; or substituting any positively charged amino acid for any other positively charge amino acid; or substituting any negatively charge amino acid for any other negatively charged amino acid (TE Creighton, “Proteins” WH Freeman and Company, New York). The amino acid substitutions include, but are not limited to, substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A), or glycine (G) and serine (S) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered conservative in particular environments.

As used herein, the term “nonconservative” refers to substituting an amino acid residue for a different amino acid residue that has different chemical properties. The nonconservative substitutions include, but are not limited to aspartic acid (D) being replaced with glycine (G); asparagine (N) being replaced with lysine (K); or alanine (A) being replaced with arginine (R).

As used herein, the term “soluble” refers to any molecule, or fragments and derivatives thereof, not bound or attached to a cell. A soluble molecule can be circulating. A soluble molecule typically lacks a transmembrane domain. A soluble molecule typically includes an extracellular domain.

The single-letter codes for amino acid residues include the following: A=alanine, R arginine, N=asparagine, D=aspartic acid, C=cysteine, Q=Glutamine, E=Glutamic acid, G=glycine, H=histidine, I=isoleucine, L=leucine, K=lysine, M=methionine, F=phenylalanine, P=proline, S=serine, T=threonine, W=tryptophan, Y=tyrosine, V=valine.

In order that the invention herein described can be more fully understood, the following description is set forth.

Molecules of the Invention

The modified hepsin molecules of the present invention include modified hepsin zymogens and activated modified hepsin molecules, or fragments or derivatives thereof. These modified hepsin molecules are useful because they comprise a substituted activation sequence that, when cleaved, activates the modified hepsin molecule. The modified hepsin molecules of the invention are stable and can be used to produce anti-hepsin antibodies.

In its various aspects, the present invention provides: modified hepsin molecules, including modified hepsin zymogens, activated modified hepsins, or fragments or derivatives thereof; nucleic acid molecules encoding the modified hepsin molecules, or fragments or derivatives thereof; recombinant DNA molecules; transformed host cells; host-vector systems; anti-hepsin antibodies; methods for using the compositions of the invention; methods for generating the compositions of the invention; assays; immunotherapeutic methods; transgenic animals; inhibitors of the modified hepsin; and immunohistochemical, immunological and nucleic acid-based assays.

Modified Hepsin Molecules

Activation of a hepsin molecule can proceed by cleavage of the peptide bond between Arg162-Ile163 in a naturally-occurring activation sequence, R-IVGG, to generate a catalytically active enzyme i.e. an active hepsin.

The modified hepsin molecules of the invention, including modified hepsin zymogens, activated modified hepsin, or fragments or derivatives thereof, comprise a substitute activation sequence.

The substitute activation sequence provides a known activation sequence in the modified hepsin molecule, which will permit cleavage of the modified hepsin molecule by a protease, e.g., a cognate protease, producing an modified activated hepsin molecule. In one embodiment, the modified hepsin molecule comprises a substitute activation sequence specifically recognized by enterokinase comprising an amino acid sequence, DDDDK, replacing amino acid Arg162. Contacting a modified hepsin molecule in its zymogen form with an enterokinase e.g. a recombinant enterokinase, cleaves the substitute activation sequence and produces a modified hepsin molecule in its activated form.

Examples of substitute activation sequences are described, infra. The sequence and length of the substitute activation sequence are selected to permit the modified hepsin zymogen to be cleaved by a desired cognate protease, thereby generating an activated modified hepsin.

The activated modified hepsin molecules of the invention exhibit the functional activity of a naturally-occurring, wild-type activated hepsin. The functional activity of a naturally-occurring wildtype activated hepsin is recognizing and cleaving the sequence, Arg-Ile, on a protein substrate, such as factor VII, to produce factor VIIa (Y Kazama, et al., 1995 J Biol Chem 270:66-72). In a similar manner, the activated modified hepsin can function as a protease and can recognize and cleave the same substrate as wildtype activated hepsin.

In accordance with the practice of the invention, modified hepsin molecules of the invention can have a folded structure which is the same, or similar to, that of naturally-occurring, wild-type hepsin molecules. For example, an activated modified hepsin a naturally-occurring, wild-type hepsin protease can be folded into a conformation that permits the catalytic site to receive and cleave a substrate recognized by wildtype activated hepsin.

A full-length, naturally-occurring, human hepsin molecule (FIG. 17) (S P Leytus, et al., 1988 Biochemistry 27:1067-1074; K Kurachi, et al., 1994 Methods Enzymol 244:100-114) includes the following: 1) a cytoplasmic domain encompassing amino acid residues 1-17; 2) a transmembrane domain encompassing amino acid residues 18-44; and 3) an extracellular domain encompassing 45-417 and comprising an activation sequence and a catalytic site.

The present invention provides modified hepsin molecules, comprising fragments or derivatives of the naturally-occurring hepsin molecules. Fragments or derivatives of the modified hepsin molecules can include any portion of the domains, described above, associated and/or linked in any combination or order.

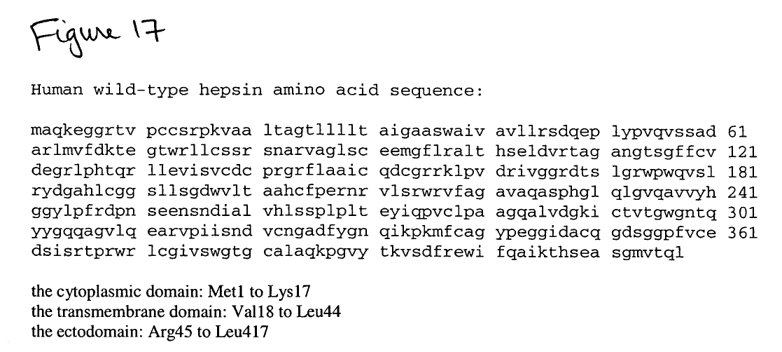

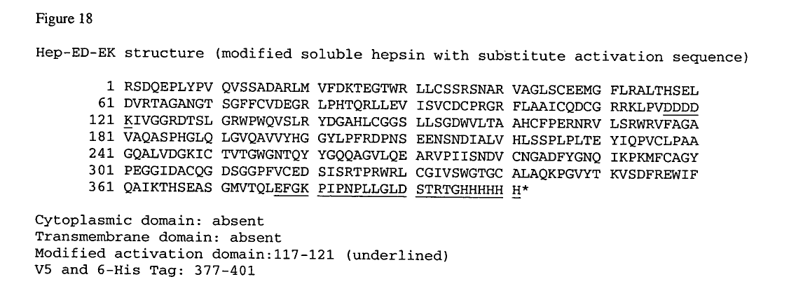

In one embodiment, a modified hepsin molecule comprises the extracellular domain of a naturally-occurring human hepsin molecule, encompassing amino acid residues 45-417 of the sequence shown in FIG. 17. In another embodiment, a modified hepsin molecule comprises the extracellular domain of a naturally-occurring hepsin molecule modified to include an enterokinase, or other protease recognition sequence, e.g., an enterokinase recognition sequence. Such embodiments are typically soluble molecules because they lack a transmembrane domain (FIG. 18).

The present invention provides modified hepsin molecules, or fragments or derivatives thereof, derived or isolated from any source whether natural, synthetic, semi-synthetic, or recombinant.

Sources include prokaryotes or eukaryotes. Eukarotic sources include animal, plants, fungi or protista. Animal sources include mammalian such bovine, porcine, murine (S Kawamura, et al., 1999 Eur J Biochem 262:755-764; D Farley, et al., 1993 Biochem Biophys Acta 1173:350-352), equine, canine, feline, simian, human (SP Leytus, et al., 1988 Biochemistry 27:1067-1074), ovine, piscine, avian or insects.

The modified hepsin molecules of the invention, or fragments or derivatives thereof, can be expressed as recombinant molecules produced in prokaryote or eukaryote host cells, or generated as synthetic molecules. In one embodiment, a recombinant modified hepsin molecule can be isolated from bacterial host cells, which produce inclusion bodies including the modified hepsin molecule (N Yamaguchi, et al. 2002, The J of Biol Chem 277:6806-6812; Takeuchi et al, 1999, PNAS 96:11054-11061). Alternative methods to isolate hepsin molecules can also be used (Wu et al, 1991, PNAS 88:6775-6779). In another embodiment, modified hepsin molecules can be isolated from baculovirus infected insect cells (Smith et al 1983 J Virol 46:584; EK Engelhard, et al, 1994 Proc Nat Acad Sci 91:3224-7).

Purification of Modified Hepsins

Modified hepsin molecules of the invention, or fragments or derivatives thereof, can be purified by methods well known in the art. These purification methods include: affinity chromatography using antibodies that selectively bind the modified hepsin molecules; affinity chromatography using antibodies that selectively bind an epitope tag linked to the modified hepsin molecules, such as His tags, V5 tags or other well known tags (Marchak, D. R., et al., 1996 in: “Strategies for Protein Purification and Characterization”, Cold Spring Harbor Press, Plainview, N.Y.); ion exchange chromatography; and gel filtration chromatography. The nature and degree of isolation and purification will depend on the intended use. For example, purified, modified hepsin molecules will be substantially free of other proteins or molecules that impair the binding of ligands or antibodies to the modified hepsin molecules.

Fusion Molecules

The present invention provides fusion molecules, or fragments or derivatives thereof, comprising a modified hepsin molecule fused to a non-hepsin molecule encoding sequence.

The fusion molecules of the invention include a modified hepsin molecule fused to an epitope tag, such as histidine (His) tags, or V5 tags, FLAG tags, influenza hemagglutinin (HA) tags, Myc tags, VSV-G tags, or thioredoxin (Trx) tags. The tagged-fusion molecules are useful for fascilitating isolation and/or purification the modified hepsin molecule (Marshak, D. R., et al., 1996 in: “Strategies for Protein Purification and Characterization” pp 396).

The fusion molecules of the invention include a modified hepsin molecule fused to a reporter molecule. The reporter molecule can be a full-length protein, or a fragment or derivative thereof. Reporter molecules commonly used include glutathione-S-transferase (GST), horseradish peroxidase (HRP), chloramphenicol acetyltransferase (CAT) beta-galactosidase, beta-glucuronidase, luciferase, green fluorescent protein (GFP), and autofluorescent proteins including blue fluorescent protein (BFP).

Other fusion molecule constructions can include maltose binding protein (MBP), S-tag, Lex A DNA binding domain (DBD) fusions, GAL4 DNA binding domain fusions, and herpes simplex virus (HSV) BP16 protein fusions.

A fusion molecule also can be engineered to include a cleavage site located between the modified hepsin molecule and the non-hepsin molecule, so that the modified hepsin molecule can be cleaved and purified away from the non-hepsin molecule. The cleavage site can include recognition sequences for the following enzymes: enterokinase, corin, MT-SP/matryptase, trypsin, chymotrypsin, human airway trypsin-like protease (HAT), mast cell tryptase, elastase, plasmin, kallikrein, TMPRSS2, MBL-associated serine proteases (MASP-1 and MASP-2), Stubble-stubbloid, furin, thrombin or factor Xa.

Chimeric Polypeptides

The present invention provides chimeric molecules, or fragments or derivatives thereof, which include a fragment of a hepsin molecule isolated from a first source fused to a fragment of a hepsin molecule isolated from a second, different source. The first and second source can be from any source including mammalian such as bovine, porcine, murine, equine, canine, feline, monkey, ape, ovine or human, or other sources such as piscine, avian or insect.

One or more of the hepsin fragments used to form a chimeric modified hepsin molecule can be modified e.g. to include an enterokinase activation sequence. In one embodiment, a chimeric, modified hepsin molecule comprises the extracellular domain of a hepsin molecule from a first source, fused to the cytoplasmic domain of a hepsin molecule from a second source, where the extracellular domain includes a substitute activation sequence. In another embodiment, a chimeric, modified hepsin molecule comprises a fragment of the extracellular domain of a hepsin molecule from a first source, fused to another fragment of the extracellular domain of a hepsin molecule from a second source, where the chimeric hepsin molecule thus formed includes a substitute activation sequence.

Amino Acid Analogs and Altered Polypeptides

The present invention further provides modified hepsin molecules, or fragments or derivatives thereof, comprising amino acid analogs. The amino acid analogs can be chemically synthesized, and include dextro or levo forms, or peptidomimetics.

The present invention also provides modified hepsin molecules which are altered, for example, by post-translational pathways or by chemical synthesis, including N- or O-glycosylated amino acid residues. The N-terminal end of the polypeptides can be altered to include acylated or alkylated residues. The C-terminal end of the polypeptides can be altered to include esterified or amidated residues. The non-terminal amino acid residues can be altered, including but not limited to, alterations of the amino acids, arginine, aspartic acid, asparagine, proline, glutamic acid, lysine, serine, threonine, tyrosine, histidine, and cysteine.

Sequence Variants

The present invention provides modified hepsin molecules, or fragments or derivatives thereof, comprising sequence variations in the extracellular domain of a naturally-occurring hepsin molecule. As persons skilled in the art understand, any number of amino acids can be varied alone, or in combination with other amino acids and yet the modified hepsin molecules will retain their functional activity (e.g., to be cleaved by a cognate protease and/or to cleave its substrates). Sequence variants of the extracellular domain of the modified hepsin molecules include: amino acid substitutions, amino acid insertions, amino acid deletions, mutant forms, allelic forms, homologs and orthologs.

Amino Acid Substitutions

The modified hepsin molecules, or fragments or derivatives thereof, can include amino acid substitutions. The extracellular domain of a modified hepsin molecule can have conservative or non-conservative amino acid substitutions. Guidance in determining which and how many amino acid residues can be substituted in the extracellular domain of the modified hepsin molecule can be found in the properties of a naturally-occuring, hepsin molecule. These properties include the amino acid length, the physical length, or in the folded conformation.

These properties can be derived by prediction (e.g., based on amino acid sequence) and/or experiment (e.g., based on X-ray crystallography). The substituted amino acids are selected so that the properties of the variant, modified hepsin molecule is identical or similar to that of a naturally-occurring hepsin molecule.

Mutant Forms

The present invention also provides modified hepsin molecules, or fragments or derivatives thereof, having a mutant form of an extracellular domain of a hepsin molecule. The mutant variant has an amino sequence that differs from that of the extracellular domain of a wild-type hepsin molecule. The mutation includes amino acid substitutions, deletions, insertions, additions, truncations, or processing or cleavage errors of the protein. The mutant variant can have the same or similar functional activity of a wild-type, hepsin molecule.

Allelic Variants

The present invention provides modified hepsin molecules, or fragments or derivatives thereof, comprising allelic variants of a naturally-occurring hepsin molecule. Allelic variants are molecules encoded by different genes residing at the same chromosomal locus.

Homologs

The present invention provides modified hepsin molecules, or fragments or derivatives thereof, comprising homologs of a naturally-occurring hepsin molecule. Homologs are molecules encoded by nucleotide sequences from the same loci but on different chromosomes. The homologs can have the same or similar functional activity.

Orthologs

The present invention provides modified hepsin molecules, or fragments or derivatives thereof, comprising orthologs of a naturally-occurring hepsin molecule. An ortholog is a hepsin molecule encoded by a nucleotide sequence from a different species. The ortholog can have the same or similar functional activity of a wild-type, hepsin molecule.

The Substitute Activation Sequence

The present invention provides modified hepsin molecules, or fragments or derivatives thereof, each including a substitute activation sequence which replaces the naturally-occurring activation sequence. The substitute activation sequence differs from the naturally-occurring activation sequence of a hepsin molecule. For example, the naturally-occurring activation sequence of a human, hepsin molecule comprises the amino acid sequence R-IVGG (A Torres-Rosado, et al., 1993 Proc Natl Acad Sci USA 90:7181-7185; K Kurachi, et al., 1994 Methods Enzymol 244:100-114). The substitute activation sequence is recognized and cleaved by a cognate protease.

The substitute activation sequence can be an amino acid sequence recognized and cleaved by a protease from any species, particularly mammalian sources, including bovine, porcine, murine, equine, canine, feline, simian, ovine or human, or other sources such as piscine, avian or insect.

The substitute activation sequence can be an amino acid sequence recognized and cleaved by a protease, including any serine protease, any member of the trypsin family, any trypsin-like protease, and any type II transmembrane protease.

The activation sequence can be an amino acid sequence recognized and cleaved by the following enzymes (A J Barrett, N D Rawlings, J F Woessner (eds), 1998, Handbook of Proteolytic Enzymes, Academic Press, London): enterokinase; thrombin; clotting factor Xa; furin; trypsin; chymotrypsin; elastase; thrombin; plasmin; kallikrein; aerosin; human airway trypsin-like protease (HAT) (K Yamaoka, et al. 1998 J Biol Chem 273:11895-11901); mast cell tryptase; MBL-associated serine proteases (MASP-1 and MASP-2) (Matsushita, et al. 2000 The Journal of Immunology 164:2281-2284); corin (W Yan, et al., 1999 J Biol Chem 274:14926-14935, W Yan, et al., 2000 Proc Natl Acad Sci USA 97:8525-8529); MT-SP1/matryptase (T Takeuchi, et al., 1999 Proc Natl Acad Sci 96:11054-11061; C Y Lin, et al., 1999 J Biol Chem 274:18231-18236); TMPRSS2 (A Paoloni-Giacobina, et al., 1997 Genomics 44:309-320); Stubble-stubbloid (L F Appel, et al., 1993 Proc Natl Acad Sci USA 90:4937-4941).

In one embodiment, the substitute activation sequence can be an amino acid sequence recognized and cleaved by a TMPRSS3 protease or an epitheliasin protease, such as the sequence LKTPR-VVGG (SEQ ID NO: 1) (A Paoloni-Giacobino, et al., 1997 Genomics 44:309-320; B Lin, et al., 1999 Cancer Res 59:4180-4184); or recognized and cleaved by an MT-SP1 protease or an epithin protease, such as the sequence TRQAR-VVGG (SEQ ID NO: 2) (M Kim, et al., 1999 Immunogenetics 49:420-428).

In another embodiment, the substitute activation sequence can be an amino acid sequence recognized and cleaved by an enterokinase protease, such as the sequence DDDDK-IVGG (SEQ ID NO: 3) (Y Kitamoto, et al., 1994 Proc Natl Acad Sci USA 91:7588-7592; ER La Vallie, et al., 1993 J Biol Chem 268:23311-23317). In another embodiment, the substitute activation sequence is recognized and cleaved by human or bovine enterokinase, comprising the amino acid sequence DDDDK-I (SEQ ID NO: 4).

Variant Substitute Activation Sequences

The present invention also provides modified hepsin molecules, or fragments or derivative thereof, comprising substitute activation sequences having sequence variations of the substitute activation sequences described above and in FIG. 18. The substitute activation sequence can have conservative amino acid substitutions, where a substituted amino acid has similar structural or chemical properties. Variants can have non-conservative changes. The variant substitute activation sequences are selected to permit the folded modified hepsin molecule to be cleaved by a cognate protease, thereby generating an activated modified hepsin molecule.

Length of the Substitute Activation Sequence

The present invention provides modified hepsin molecules comprising a substitute activation sequence ranging in size between about 2 to about 10 amino acid residues in length. In one embodiment, the substitute activation sequence is about 2 to about 6 amino acids in length.

The substitute activation sequence can be selected to span the same or similar distance of the activation sequence in a folded wild-type hepsin molecule. In one embodiment, the substitute activation sequence will not affect the functional activity of the modified hepsin molecule.

Guidance in determining which and how many amino acid residues can be varied in the substitute activation sequence can be found in the distance spanned by the activation sequence in a folded modified hepsin molecule. The distance that spans the activation sequence in a folded wild-type hepsin molecule can be predicted from the amino acid sequence of a wild-type hepsin molecule and/or obtained experimentally from X-ray crystal structures of a wild-type hepsin molecule.

For example, the amino acid sequence of wild-type, human hepsin (A Torres-Rosado, et al., 1993 Proc Natl Acad Sci USA 90:7181-7185; K Kurachi, et al., 1994 Methods Enzymol 244:100-114) can be used as a basis to predict the distance that spans the activation sequence in a folded human wild type hepsin molecule. The activation sequence of wild-type human hepsin molecule, encompassing residues RIVGG, spans a linear length of five amino acid residues (FIG. 17).

Nucleic Acid Molecules of the Invention

Nucleic Acid Molecules Encoding Modified Hepsin Molecules

The present invention provides various isolated, and recombinant nucleic acid molecules, or fragments or derivatives thereof, comprising polynucleotide sequences encoding the modified hepsin molecules of the invention, are herein referred to as “modified hepsin polynucleotide sequences,” “hepsin sequences”, “hepsin molecule sequences” or “nucleic acid molecules of the invention”. The present invention also provides polynucleotide sequences that encode a fragment or derivative of the modified hepsin molecules. The present invention further provides related polynucleotide molecules, such as complementary modified hepsin polynucleotide sequences, or a part thereof, and those that hybridize to the nucleic acid molecules of the invention.

The modified hepsin polynucleotide sequences, are preferably in isolated form, and include, but are not limited to, DNA, RNA, DNA/RNA hybrids, and related molecules, and fragments thereof. Specifically contemplated are genomic DNA, cDNA, ribozymes, and antisense RNA or DNA molecules, as well as nucleic acid molecules based on an alternative backbone or including alternative bases, whether derived from natural sources or synthesized.

The nucleic acid molecules of the invention encode the modified hepsin molecules of the invention and/or fragments or derivatives thereof, where the encoded modified hepsin molecule exhibits similar or identical functional activity of a naturally-occurring hepsin molecule.

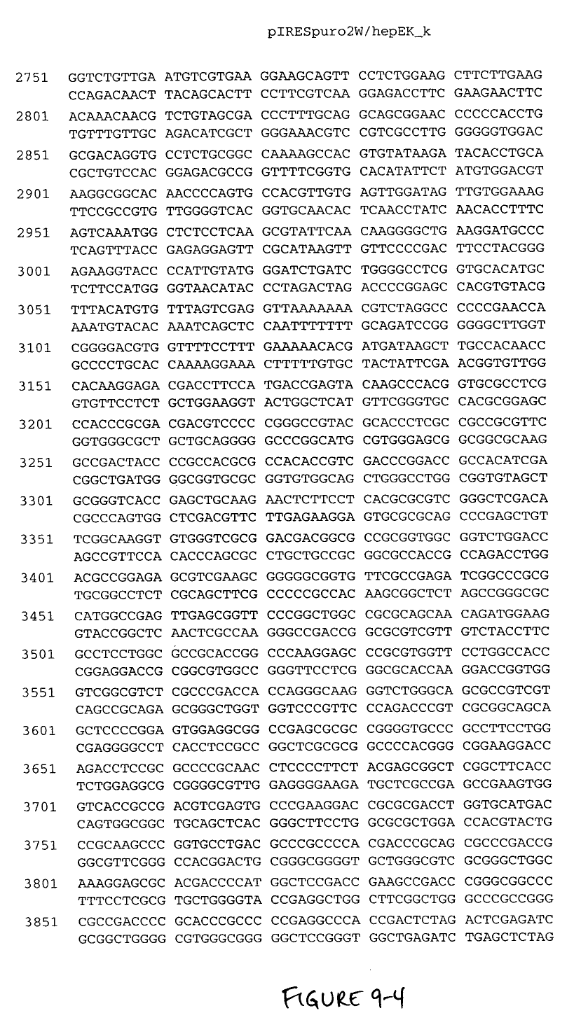

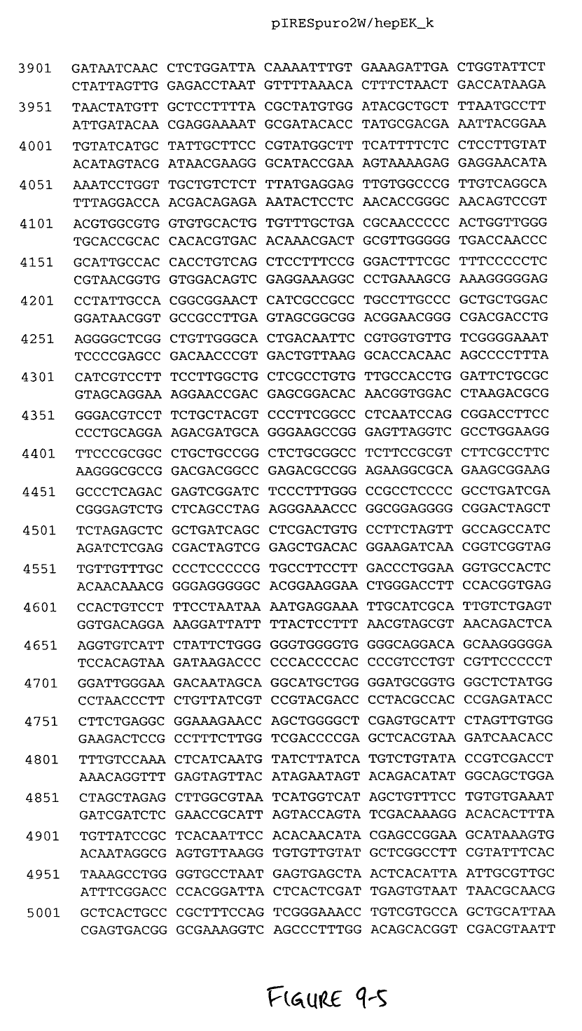

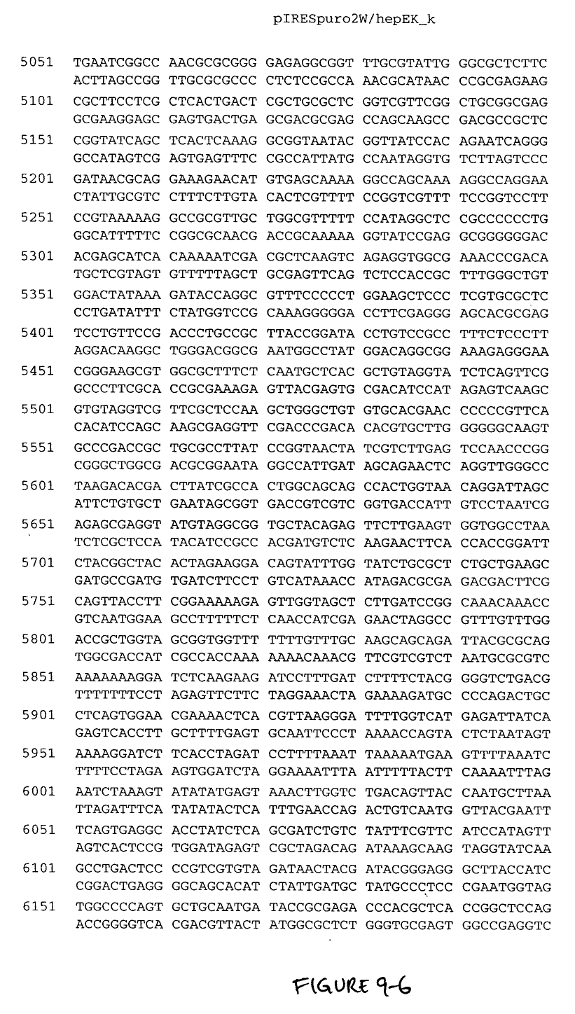

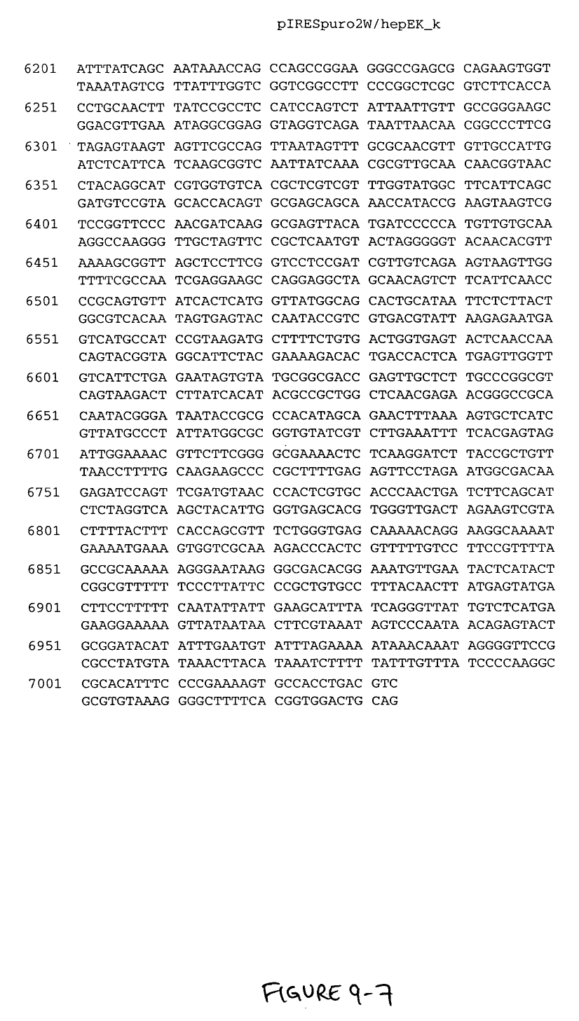

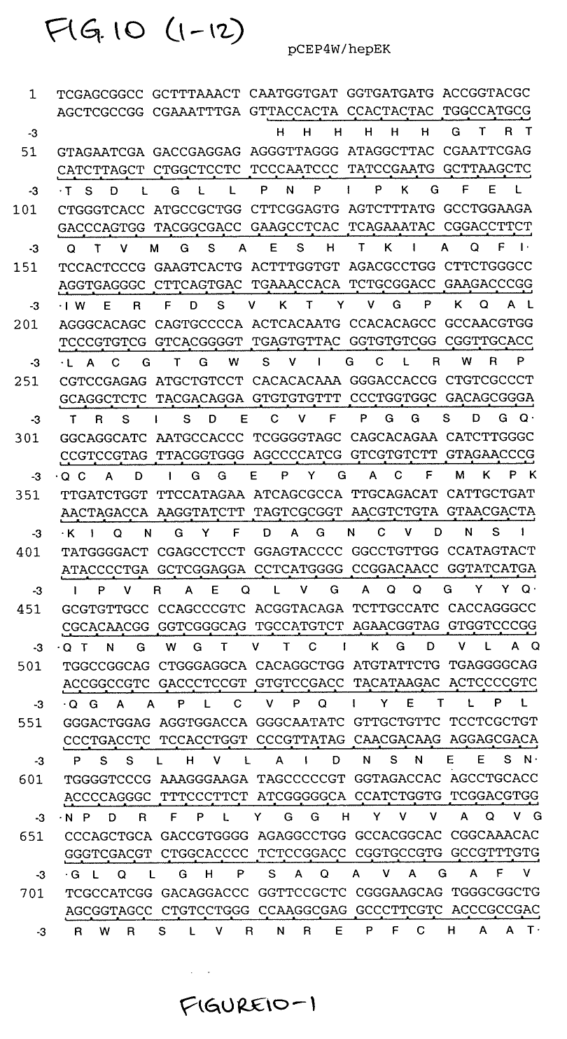

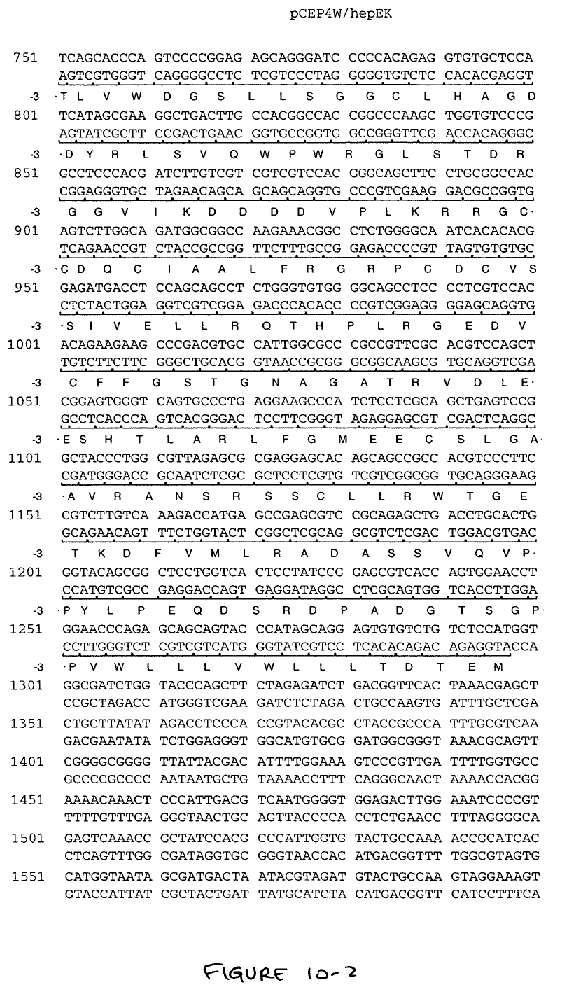

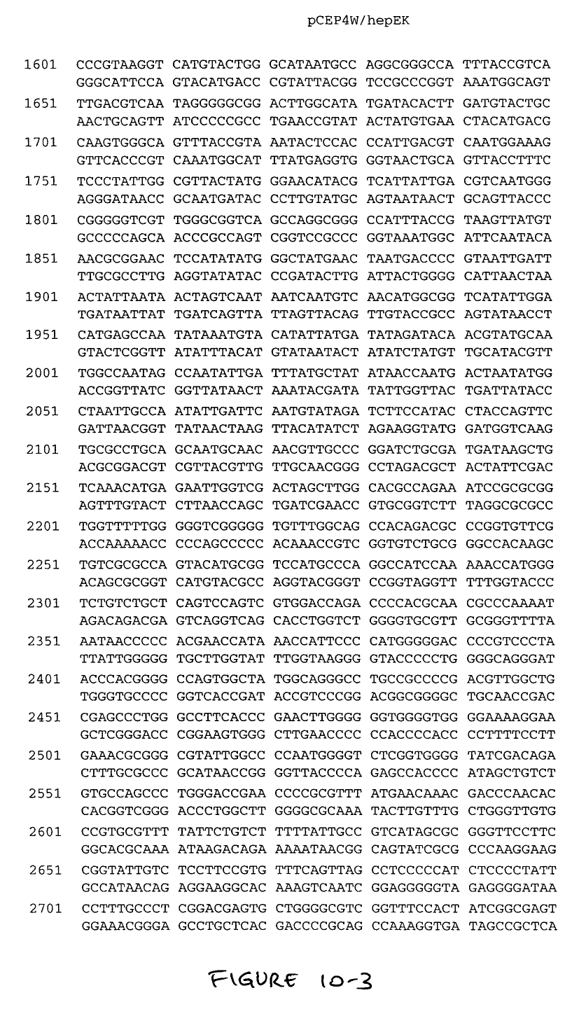

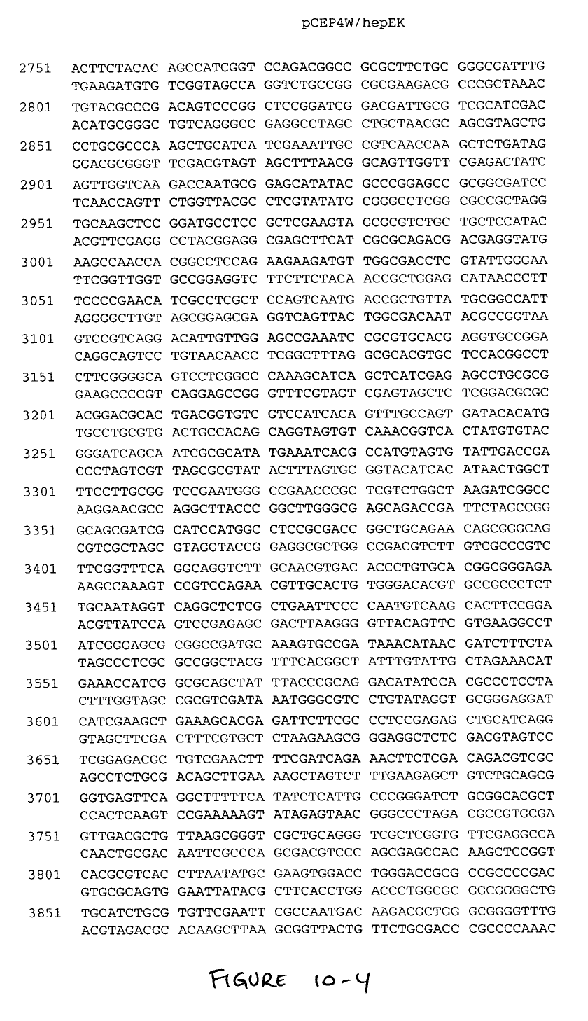

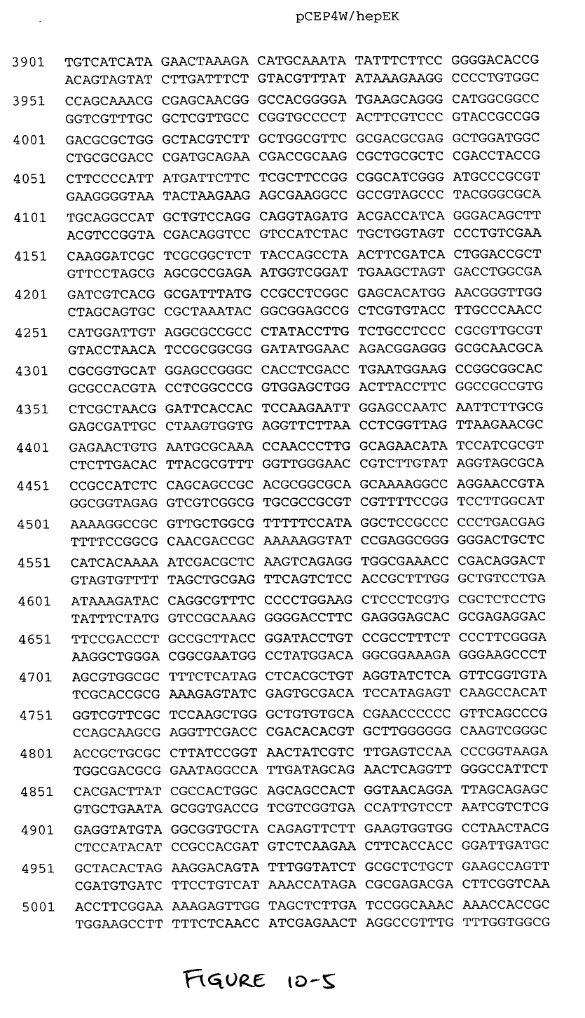

In one embodiment, an isolated nucleotide sequence encoding a modified hepsin molecule is shown in FIG. 9, beginning at codon agg at position 996 and ending at codon ctc at position 2123. Additionally, the nucleic acid sequence of FIG. 9 encodes a signal sequence for protein secretion at position 924-995 and encodes a V5 and 6-His tag sequence at position 2124-2198.

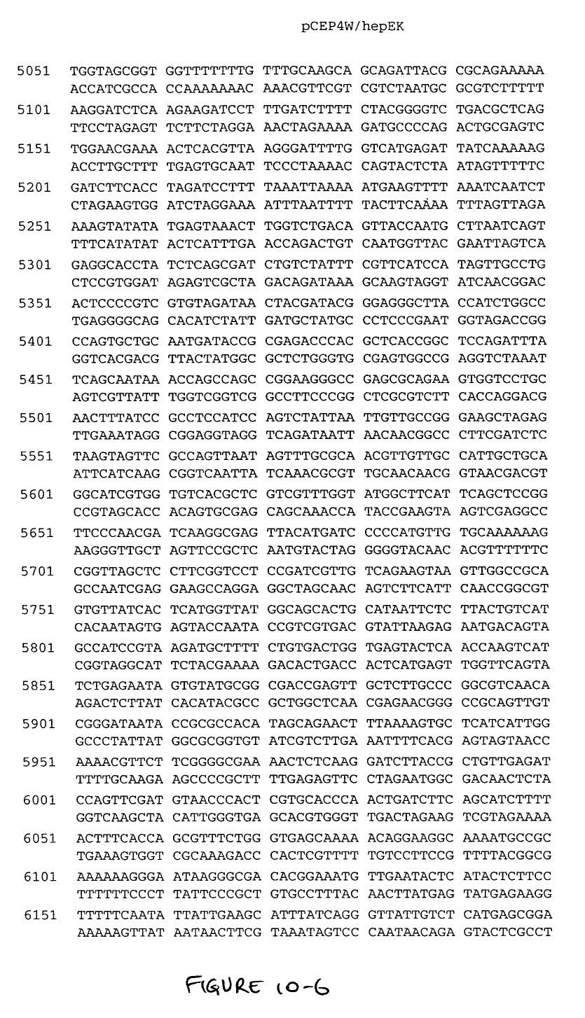

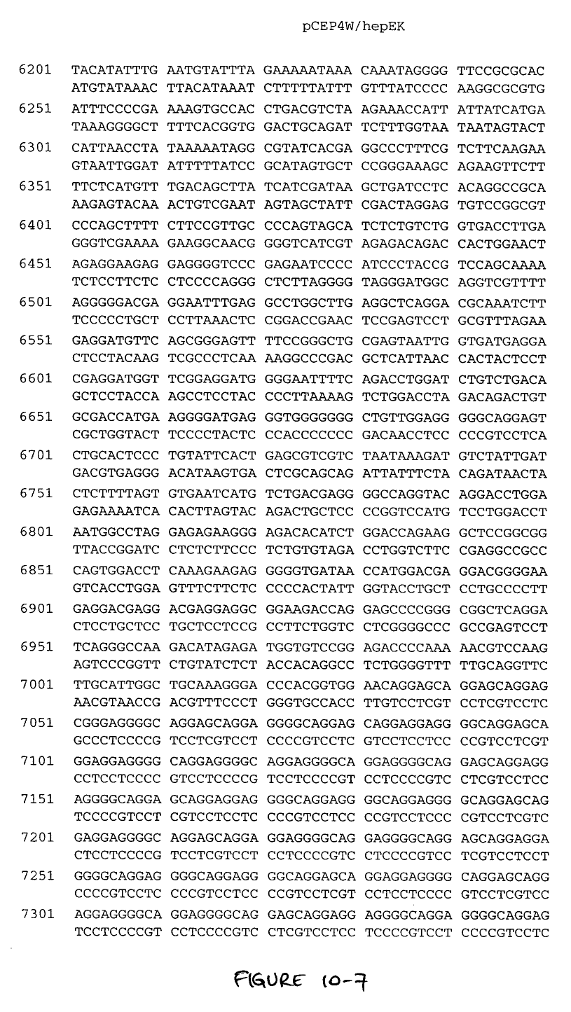

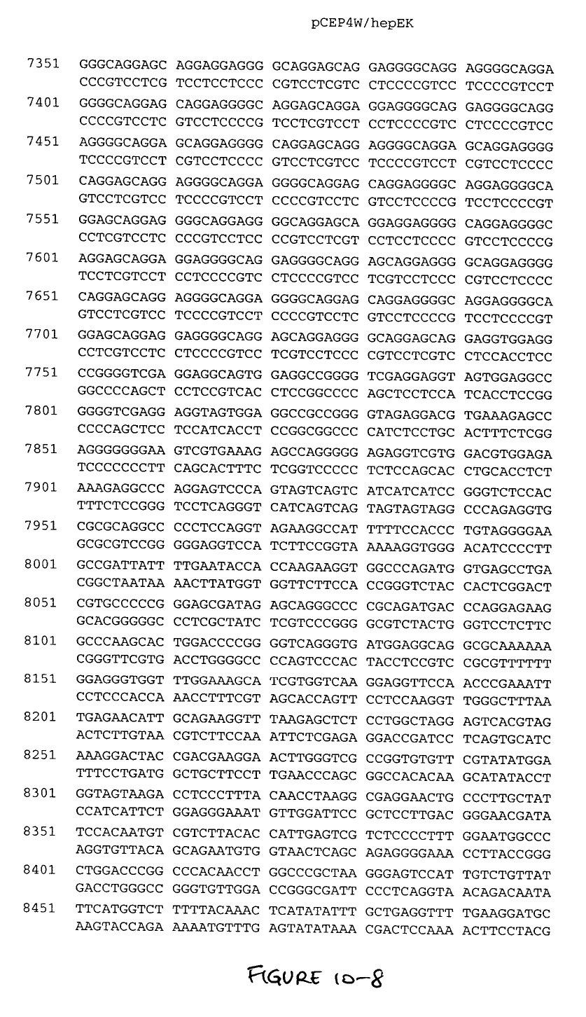

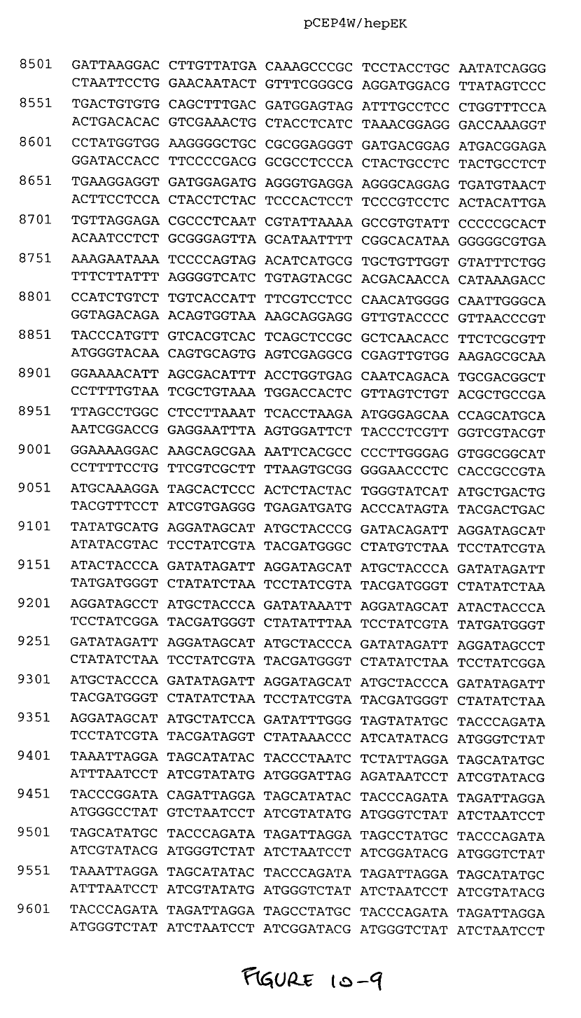

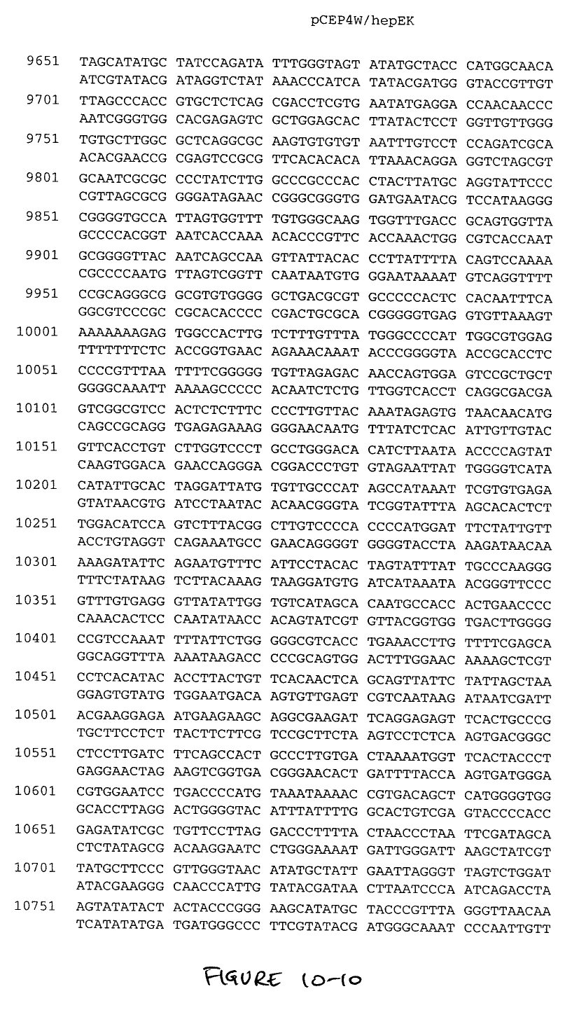

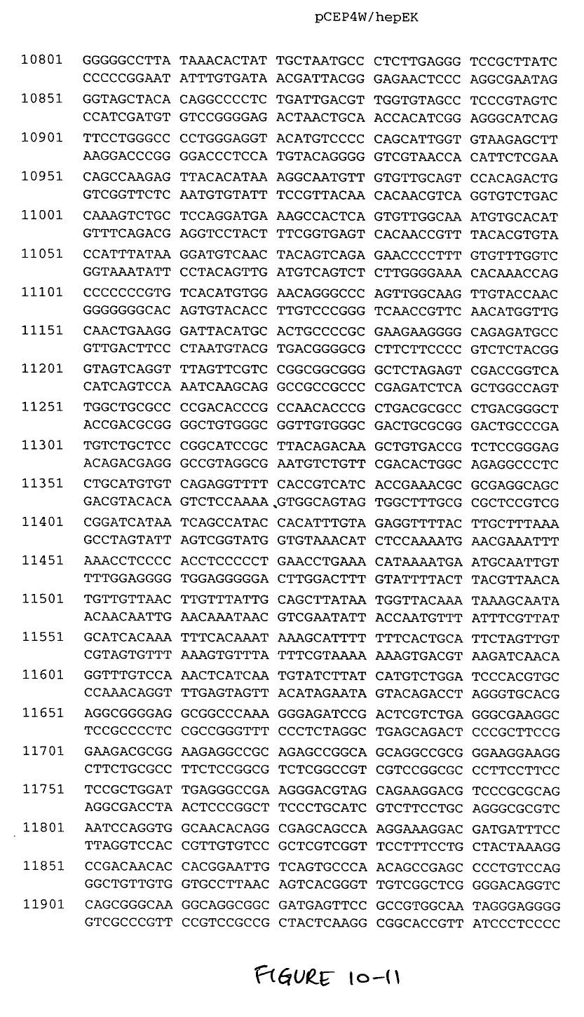

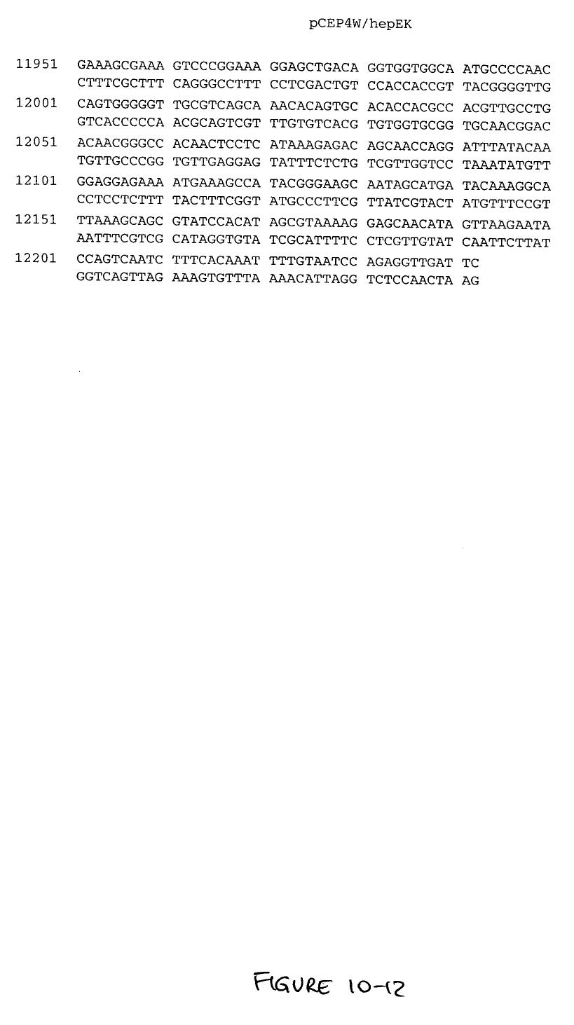

In another embodiment, the isolated hepsin sequence shown in FIG. 10 encodes a modified hepsin molecule, beginning with a agg at position 1225 and ending with ctc at position 98. Nucleic acid sequence 1297-1226 of FIG. 10 encodes a signal sequence and sequence 97-23 encodes a V5 and 6-His Tag sequence.

The isolated hepsin sequence shown in FIG. 11 encodes a modified hepsin molecule, beginning with caa at position 907 and ending with ctc at position 98. Nucleic acid sequence 979-908 encodes a signal sequence and sequence 97-23 encodes a V5 and 6-His Tag sequence.

A biological sample of the nucleotide sequence shown in FIG. 10, and designated pCEP4W/hepEK was deposited on Sep. 30, 2002, with the American Type Culture Collection (ATCC), 10801 University Blvd., Manassas, Va. 20110-2209 under the provisions of the Budapest Treaty, and has been accorded ATCC accession number (PTA-4733).

In accordance with the practice of the invention, the nucleic acid molecules of the invention can be isolated full-length or partial length molecules or oligomers of the modified hepsinnucleotide sequences. The hepsin sequence of the invention can encode all or portions of the modified hepsin molecules of the invention, including the cytoplasmic domain, transmembrane domain, and/or the extracellular domain. The extracellular domain comprises a substitute activation sequence.

Isolated Nucleic Acid Molecules

The nucleic acid molecules of the invention are preferably in isolated form, where the nucleic acid molecules are substantially separated from contaminant nucleic acid molecules having sequences other than modified hepsin molecule sequences. A skilled artisan can readily employ nucleic acid isolation procedures to obtain isolated, modified hepsin molecule sequences, see for example Sambrook et al., in: “Molecular Cloning” (1989). The present invention also provides for isolated modified hepsin molecule sequences generated by recombinant DNA technology or chemical synthesis methods. The present invention also provides nucleotide sequences isolated from various mammalian species including, bovine, porcine, murine, equine, canine, feline, simian, ovine or human, or other sources such as piscine, avian or insect.

The isolated nucleic acid molecules include DNA, RNA, DNA/RNA hybrids, and related molecules, nucleic acid molecules complementary to the modified hepsin molecule nucleotide sequence encoding a modified hepsin molecule, or a fragment or derivative thereof, and those which hybridize to the nucleic acid molecules that encode the modified hepsin molecules. The preferred nucleic acid molecules have nucleotide sequences identical to or similar to the nucleotide sequences disclosed herein. Specifically contemplated are genomic DNA, RNA e.g., small interfering RNA, cDNA, ribozymes and antisense molecules.

Identical and Similar Nucleotide Sequences

The present invention provides isolated nucleic acid molecules having a polynucleotide sequence identical or similar to the modified hepsin molecule sequences disclosed herein. Accordingly, the polynucleotide sequences can be identical to a particular modified hepsin molecule sequence, as described in FIGS. 9-11. Alternatively, the polynucleotide sequences can be similar to the disclosed sequences.

One embodiment of the invention provides nucleic acid molecules that exhibit sequence identity or similarity with the modified hepsin molecule nucleotide sequences, such as molecules that have at least 60% to 99.9% sequence similarity and up to 100% sequence identity with the sequences of the invention as shown in FIGS. 9-11. Another embodiment provides nucleic acid molecules that exhibit between about 75% to 99.9% sequence similarity, and another embodiment provides molecules that have between about 86% to 99.9% sequence similarity. Yet another embodiment provides molecules that have 100% sequence identity with the modified hepsin molecule sequences of the invention as shown in FIGS. 9-11.

Complementary Nucleotide Sequences

The present invention also provides nucleic acid molecules that are complementary to the sequences as described in FIGS. 9-11, 17-18. Complementarity can be full or partial. A nucleotide sequence that is fully complementary is complementary to the entire hepsin sequence as described in any one of FIGS. 9-11 and 17-18. A nucleotide sequence that is partially complementary is complementary to only a portion of sequences as described in any one of FIGS. 9-11 and 17-18. The complementary molecules include anti-sense nucleic acid molecules. The anti-sense molecules are useful for RNA interference (RNAi), DNA interference, inhibiting growth of a cell or killing a cell expressing a naturally-occurring hepsin molecule or expressing a modified hepsin molecule (A Torres-Rosado, et al., 1993 Proc Natl Acad Sci USA 90:7181-7185). The complementary molecules also include small interfering RNA (siRNA) (Elbashir et al, 2001, Nature 411:494-498; Hammond et al, 2001, Nature Review 2:110-119).

Hybridizing Nucleic Acid Molecules

The present invention further provides nucleic acid molecules having polynucleotide sequences that selectively hybridize to the modified hepsin molecule nucleotide sequence of the invention as shown in any one of FIGS. 9-11 and 17-18. The nucleic acid molecules that hybridize can hybridize under high stringency hybridization conditions. Typically, hybridization under standard high stringency conditions will occur between two complementary nucleic acid molecules that differ in sequence complementarity by about 70% to about 100%. It is readily apparent to one skilled in the art that the high stringency hybridization between nucleic acid molecules depends upon, for example, the degree of identity, the stringency of hybridization, and the length of hybridizing strands. The methods and formulas for conducting high stringency hybridizations are well known in the art, and can be found in, for example, Sambrook, et al., in: “Molecular Cloning” (1989).

In general, stringent hybridization conditions are those that: (1) employ low ionic strength and high temperature for washing, for example, 0.015M NaCl/0.0015M sodium titrate/0.1% SDS at 50 degrees C.; or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42 degrees C.

Another example of stringent conditions include the use of 50% formamide, 5×SSC (0.75M NaCl, 0.075 M sodium citrate), 50 mM sodium phosphate (pH 6.8), 0.1% sodium pyrophosphate, 5× Denhardt's solution, sonicated salmon sperm DNA (50 mg/ml), 0.1% SDS, and 10% dextran sulfate at 42 degrees C., with washes at 42 degrees C. in 0.2×SSC and 0.1% SDS. A skilled artisan can readily determine and vary the stringency conditions appropriately to obtain a clear and detectable hybridization signal.

Nucleic Acid Fragments

The present invention further provides nucleic acid molecules having fragments of the modified hepsin molecule sequences of the invention, such as a portion of the modified hepsin molecule sequences disclosed herein and as shown in any one of FIGS. 9-11 and 18. The size of the fragment will be determined by its intended use. For example, if the fragment is chosen to encode a modified hepsin molecule comprising the extracellular domain of a naturally-occurring, wild-type hepsin molecule comprising a substitute activation sequence, then the skilled artisan shall select the polynucleotide fragment that is large enough to encode this domain(s). If the fragment is to be used as a nucleic acid probe or PCR primer, then the fragment length is chosen to obtain a relatively small number of false positives during a probing or priming procedure.

The nucleic acid molecules, fragments thereof, and probes and primers of the present invention are useful for a variety of molecular biology techniques including, for example, hybridization screens of libraries, or detection and quantification of mRNA transcripts as a means for analysis of gene transcription and/or expression. The probes and primers can be DNA, RNA or derivatives of DNA or RNA molecules. A probe or primer length of at least 15 base pairs is suggested by theoretical and practical considerations (Wallace, B. and Miyada, G. 1987 in: “Oligonucleotide Probes for the Screening of Recombinant DNA Libraries” in: Methods in Enzymology, 152:432-442, Academic Press).

Fragments of the modified hepsin molecule nucleotide sequences that are particularly useful as selective hybridization probes or PCR primers can be readily identified from the modified hepsin molecule nucleotide sequences, using art-known methods. For example, sets of PCR primers that bind and/or detect a portion of modified hepsin molecule transcripts can be made by the PCR method described in U.S. Pat. No. 4,965,188. The probes and primers of this invention can be prepared by methods well known to those skilled in the art (Sambrook, et al. supra). The probes and primers can be synthesized by chemical synthesis methods (ed: Gait, M. J. 1984 in: “Oligonucleotide Synthesis”, IRL Press, Oxford, England).

One embodiment of the present invention provides nucleic acid primers that are complementary to the modified hepsin molecule sequences, which allow specific amplification of nucleic acid molecules of the invention or of any specific portions thereof. Another embodiment provides nucleic acid probes that are complementary for selectively or specifically hybridizing to the modified hepsin molecule sequences or to any portion thereof.

Alternatively, a fragment of the modified hepsin molecule sequence can be used to construct a recombinant fusion gene having a modified hepsin molecule sequence fused to a non-hepsin molecule sequence.

Fusion Gene Sequences

The present invention provides fusion gene sequences, which include a modified hepsin molecule sequence fused (e.g., linked or joined) to a non-hepsin molecule sequence. The modified hepsin molecule sequence is operatively fused, in-frame, to a non-hepsin molecule sequence.

The fusion gene sequences of the invention include a nucleotide sequence encoding modified hepsin molecule fused to an epitope tag, including but not limited to, histidine (His) tags, V5 tags, FLAG tags, influenza hemagglutinin (HA) tags, Myc tags, VSV-G tags, and thioredoxin (Trx) tags.

The fusion gene sequences of the invention include a nucleotide sequence encoding modified hepsin molecule fused to a full-length or partial-length reporter gene sequence, including but not limited to glutathione-S-transferase (GST), horseradish peroxidase (HRP), chloramphenicol acetyltransferase (CAT) beta-galactosidase, beta-glucuronidase, luciferase, green fluorescent protein (GFP), and autofluorescent proteins including blue fluorescent protein (BFP).

The fusion gene sequences of the invention include a nucleotide sequence encoding modified hepsin molecule fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions.

The fusion gene sequences of the invention include a nucleotide sequence encoding modified hepsin molecule fused to a gene sequence encoding a cleavage site moiety. The cleavage site can be located between the modified hepsin molecule-encoding sequence and the cleavage sequence. The cleavage site moiety includes, but is not limited to hepsin, thrombin, and factor Xa recognition sequences.

Chimeric Nucleotide Sequences

The present invention provides chimeric gene sequences encoding recombinant, chimeric modified hepsin molecules. The chimeric nucleotide molecules encode a portion of a hepsin molecule isolated from a first source fused to a portion of a hepsin molecule isolated from a second, different source. The chimeric molecules encode chimeric polypeptides operatively fused, in-frame.

In one example, a chimeric nucleotide molecule encodes the extracellular domain of a hepsin molecule from a first source, fused to the cytoplasmic domain of a hepsin molecule from a second source, where the extracellular domain includes a substitute activation sequence. In another example, a chimeric nucleotide molecule encodes a portion of the extracellular domain of a hepsin molecule from a first source, fused to the remaining portion of the extracellular domain of a hepsin molecule from a second source, where the chimeric molecule includes an extracellular domain having a substitute activation sequence.

Codon Usage Variants

The present invention provides isolated codon-usage variants that differ from the disclosed modified hepsin molecule nucleotide sequences, yet do not alter the predicted polypeptide sequence or biological activity of the encoded modified hepsin molecule. For example, a number of amino acids are designated by more than one triplet codon. Codons that specify the same amino acid can occur due to degeneracy in the genetic code. Examples include nucleotide codons CGT, CGG, CGC, and CGA encoding the amino acid, arginine (R); or codons GAT, and GAC encoding the amino acid, aspartic acid (D). Thus, a protein can be encoded by one or more nucleic acid molecules that differ in their specific nucleotide sequence, but still encode protein molecules having identical sequences. The amino acid coding sequence is as follows:

| | | One | | |
|---|---|---|---|---|
| Amino Acid | Symbol | Symbol | Letter | Codons |
| Alanine | Ala | A | | GCU, GCC, GCA, GCG |
| Cysteine | Cys | C | | UGU, UGC |
| Aspartic Acid | Asp | D | | GAU, GAC |
| Glutamic Acid | Glu | E | | GAA, GAG |
| Phenylalanine | Phe | F | | UUU, UUC |
| Glycine | Gly | G | | GGU, GGC, GGA, GGG |
| Histidine | His | H | | CAU, CAC |
| Isoleucine | Ile | I | | AUU, AUC, AUA |
| Lysine | Lys | K | | AAA, AAG |
| Leucine | Leu | L | | UUA, UUG, CUU, CUC, |
| | | | | CUA, CUG |
| Methionine | Met | M | | AUG |
| Asparagine | Asn | N | | AAU, AAC |
| Proline | Pro | P | | CCU, CCC, CCA, CCG |
| Glutamine | Gln | Q | | CAA, CAG |
| Arginine | Arg | R | | CGU, CGC, CGA, CGG, |
| | | | | AGA, AGG |
| Serine | Ser | S | | UCU, UCC, UCA, UCG, |
| | | | | AGU, AGC |
| Threonine | Thr | T | | ACU, ACC, ACA, ACG |
| Valine | Val | V | | GUU, GUC, GUA, GUG |
| Tryptophan | Trp | W | | UGG |
| Tyrosine | Tyr | Y | | UAU, UAC |

The codon-usage variants can be generated by recombinant DNA technology. Codons can be selected to optimize the level of production of the modified hepsin molecule transcript or the modified hepsin molecule in a particular prokaryotic or eukaryotic expression host, in accordance with the frequency of codon utilized by the host cell. Alternative reasons for altering the nucleotide sequence encoding a modified hepsin molecule include the production of RNA transcripts having more desirable properties, such as an extended half-life or increased stability. A multitude of variant modified hepsin molecule nucleotide sequences that encode the respective modified hepsin molecule can be isolated, as a result of the degeneracy of the genetic code. Accordingly, the present invention provides selecting every possible triplet codon to generate every possible combination of nucleotide sequences that encode the disclosed modified hepsin molecule, or that encode molecules having the biological activity of the modified hepsin molecule. This particular embodiment provides isolated nucleotide sequences that vary from the sequences as described in described in any one of FIGS. 9-11 and 17-18, such that each variant nucleotide sequence encodes a molecule having sequence identity with the amino acid sequence described in FIGS. 9-11 and 17-18.

Variant Nucleotide Sequences

The present invention provides nucleic acid molecules comprising polynucleotide sequences encoding variant forms of any of the modified hepsin molecules of the invention. The variant nucleotide sequences encode variant forms of the extracellular domain of the modified hepsin molecule. The variant nucleotide sequences encode variant forms of the substitute activation sequence within the modified hepsin molecules of the invention. In one embodiment, the variant nucleotide sequence encodes a variant modified hepsin molecule having the same or similar functional activity of a naturally-occurring, wild-type hepsin molecule.

The variant nucleotide sequences of the present invention include conservative or non-conservative amino acid substitutions. The variant nucleotide sequences include mutations such as amino acid substitutions, deletions, insertions, additions, truncations, or processing or cleavage errors of the protein. The variant nucleotide sequences include allelic, homolog, or ortholog variants of the naturally-occurring hepsin molecule.

Derivative Nucleic Acid Molecules

The nucleic acid molecules of the invention also include derivative nucleic acid molecules which differ from DNA or RNA molecules, and anti-sense molecules. Derivative molecules include peptide nucleic acids (PNAs), and non-nucleic acid molecules including phosphorothioate, phosphotriester, phosphoramidate, and methylphosphonate molecules, that bind to single-stranded DNA or RNA in a base pair-dependent manner (P C Zamecnik, et al., 1978 Proc. Natl. Acad. Sci. 75:280284; P C Goodchild, et al., 1986 Proc. Natl. Acad. Sci. 83:4143-4146). Peptide nucleic acid molecules comprise a nucleic acid oligomer to which an amino acid residue, such as lysine, and an amino group have been added. These small molecules, also designated anti-gene agents, stop transcript elongation by binding to their complementary (template) strand of nucleic acid (P E Nielsen, et al., 1993 Anticancer Drug Des 8:53-63). Reviews of methods for synthesis of DNA, RNA, and their analogues can be found in: Oligonucleotides and Analogues, eds. F Eckstein, 1991, IRL Press, New York; Oligonucleotide Synthesis, ed. M J Gait, 1984, IRL Press, Oxford, England. Additionally, methods for antisense RNA technology are described in U.S. Pat. Nos. 5,194,428 and 5,110,802. A skilled artisan can readily obtain these classes of derivative nucleic acid molecules using the herein described modified hepsin molecule polynucleotide sequences, see for example “Innovative and Perspectives in Solid Phase Synthesis” (1992) Egholm, et al. pp 325-328 or U.S. Pat. No. 5,539,082.

Labeled Nucleic Acid Molecules

The present invention provides nucleic acid molecules of the invention linked or labeled with a detectable marker. Examples of a detectable marker include, but are not limited to, a radioisotope, a fluorescent compound, a bioluminescent compound, a chemiluminescent compound, a metal chelator or an enzyme. Technologies for generating labeled nucleic acid molecules are well known, see, for example, Sambrook et al., in Molecular Cloning (1989).

Recombinant Nucleic Acid Molecules

The present invention provides recombinant DNA molecules (rDNAs) that include nucleotide sequences encoding modified hepsin molecules, or a fragment or derivative thereof, as described herein. As used herein, a rDNA molecule is a DNA molecule that has been subjected to molecular manipulation in vitro. Methods for generating recombinant DNA molecules are well known in the art, for example, see Sambrook et al., Molecular Cloning (1989). In one embodiment, the recombinant DNA molecules of the present invention are operably linked to one or more expression control sequences and/or vector sequences.

Vectors

The nucleic acid molecules of the invention can be recombinant molecules each comprising the polynucleotide sequence, or fragments or derivatives thereof, encoding a modified hepsin molecule linked to a vector to generate a recombinant vector molecule.

The term vector includes, but is not limited to, plasmids, cosmids, BACs, YACs PACs and phagemids. The vector can be an autonomously replicating vector comprising a replicon that directs the replication of the rDNA within the appropriate host cell. Alternatively, the vector directs integration of the recombinant vector into the host cell. Various viral vectors can also be used, such as, for example, a number of well known retroviral and adenoviral vectors (Berkner 1988 Biotechniques 6:616-629).

The vectors of the invention permit expression of the modified hepsin molecule, or fragments or derivatives thereof, in prokaryotic or eukaryotic host cells. The vectors can be expression vectors, comprising an expression control element, such as a promoter sequence, which enables transcription of the inserted modified hepsin molecule nucleotide sequence and can be used for regulating the expression (e.g., transcription and/or translation) of a linked modified hepsin molecule sequence in an appropriate host cell.

The expression control elements can be of various origins, including naturally-occurring and synthetic. The naturally-occurring elements can be cellular or viral in origin. Expression control elements are known in the art and include, but are not limited to, inducible promoters, constitutive promoters, secretion signals, enhancers, transcription terminators, and other transcriptional regulatory elements.

Other expression control elements that are involved in translation are known in the art, and include the Shine-Dalgarno sequence (e.g., prokaryotic host cells), and initiation and termination codons. Exogenous transcriptional elements and initiation codons can be of various origins, both natural and synthetic.

The promoters can be inducible which are regulated by environmental stimuli or the growth medium of the cells, including those from the genes for heat shock proteins, alcohol dehydrogenase 2, isocytochrome C, acid phosphatase, enzymes associated with nitrogen catabolism, and enzymes responsible for maltose and galactose utilization.

The promoters can be constitutive including yeast beta-factor, alcohol oxidase, cytomegalovirus, and PGH. For reviews, see Ausubel et al (1987 Current Protocols in Molecular Biology, John Wiley & Sons, New York N.Y.) and Grant et al (1987 Methods in Enzymology 153:516-544).

The efficiency of transcription can be augmented by the inclusion of enhancers appropriate to the cell system in use (Scharf, D., et al, 1994 Results Probl. Cell. Differ. 20:125-62; Bittner, et al., 1987 Methods in Enzymol. 153:516-544). Viral promoters include SV40 early promoter or the promoter included within the LTR of a retroviral vector. Other viral promoters include the cytomegalovirus promoter (M Boshart, et al., 1985 Cell 41:521-530).

Commonly used eukaryotic control sequences for use in expression vectors include promoters and control sequences compatible with mammalian cells such as, for example, CMV promoter and avian sarcoma virus (ASV) (πLN vector). Other commonly used promoters include the early and late promoters from Simian Virus 40 (SV40) (Fiers, et al., 1973 Nature 273:113), or other viral promoters such as those derived from polyoma, Adenovirus 2, and bovine papilloma virus. An inducible promoter, such as hMTII (Karin, et al., 1982 Nature 299:797-802) can also be used.

Transcriptional control sequences for yeast vectors include promoters for the synthesis of glycolytic enzymes (Hess et al., 1968) J Adv Enzyme Reg. 7:149; Holland et al., 1978 Biochemistry 17:4900). Additional promoters known in the art include the CMV promoter provided in the CDM8 vector (Toyama and Okayama 1990 FEBS 268:217-221); the promoter for 3-phosphoglycerate kinase (Hitzeman et al., 1980 J Biol Chem 255:2073), and those for other glycolytic enzymes.

Specific translation initiation signals can also be required for efficient translation of a modified hepsin molecule sequence. These signals include the ATG-initiation codon and adjacent sequences. The ATG-initiation sequences or upstream sequences of a naturally-occurring hepsin molecule can be inserted into the appropriate expression vector. Alternatively, a synthetic ATG-initiation codon and other sequences can be used. The ATG-initiation codon must be in the correct reading-frame to ensure translation of the insert sequence.

The expression control elements can be placed at the 3′ end of the coding sequences. These sequences can act to stabilize messenger RNA. Such terminators are found in the 3′ untranslated region following the coding sequences in several yeast-derived and mammalian genes.

The expression vector can include at least one selectable marker gene encoding a gene product that confers drug resistance such as resistance to kanamycin, ampicillin or tetracyline.

The expression vector can include any marker gene. These include, but are not limited to, the herpes simplex virus thymidine kinase (M Wigler et al., 1977 Cell 11:223-32) and adenine phosphoribosyltransferase (I Lowy et al., 1980 Cell 22:817-23) genes which can be employed in tk-minus or aprt-minus cells, respectively. Also, antimetabolite, antibiotic or herbicide resistance can be used as the basis for selection; for example, dhfr which confers resistance to methotrexate (M Wigler et al., 1980 Proc Natl Acad Sci 77:3567-70); npt, which confers resistance to the aminoglycosides neomycin and G-418 (F Colbere-Garapin et al., 1981 J. Mol. Biol. 150:1-14) and als or pat, which confer resistance to chlorsulfuron and phosphinotricin acetyltransferase, respectively (L E Murry, in: McGraw Yearbook of Science and Technology (1992) McGraw Hill New York N.Y., pp 191-196). Additional selectable genes have been described, for example, trpB, which allows cells to utilize indole in place of tryptophan, or hisD, which allows cells to utilize histinol in place of histidine (Hartman, and Mulligan 1988 Proc. Natl. Acad. Sci. 85:8047-51). Recently, the use of visible markers has gained popularity with such markers as anthocyanins, β-glucuronidase and its substrate, GUS, and luciferase and its substrate, luciferin, being widely used not only to identify transformants, but also to quantify the amount of transient or stable protein expression attributable to a specific vector system (C A Rhodes et al., 1995 Methods Mol. Biol. 55:121-131).

The vector also comprises multiple endonuclease restriction sites that enable convenient insertion of exogenous DNA sequences. Methods for generating a recombinant expression vector encoding the modified hepsin molecules of the invention are well known in the art (T Maniatis, et al., 1989 Molecular Cloning, A Laboratory Manual, Cold Spring Harbor Laboratory, Cold Spring Harbor, N.Y.; F Ausubel, et al. 1989 Current Protocols in Molecular Biology, John Wiley & Sons, New York N.Y.).

The expression vectors used for generating modified hepsin molecules are compatible with eukaryotic host cells. The vectors can be compatible with vertebrate cells. These vectors can include expression control elements such as promoters and/or enhancers from mammalian genes or mammalian viruses. Other expression vectors can include tissue- or cell-specific promoters and/or enhancers from mammalian genes or mammalian viruses.

The expression vectors can be compatible with other eukaryotic host cells, including insect, plant, or yeast cells. The expression vectors can include expression control elements, such as the baculovirus polyhedrin promoter for expression in insect cells. The promoters and/or enhancers derived from plant cells (e.g., heat shock, RUBISCO, storage protein genes), viral promoters or leader sequences or from plant viruses can also be used.

Eukaryotic cell expression vectors are well known in the art and are available from several commercial sources, including PSVL and pKSV-10 (Pharmacia), pBPV-1/pML2d (International Biotechnologies, Inc.), pTDT1 (ATCC, #31255), and similar eukaryotic expression vectors. Examples of expression vectors for eukaryotic host cells include, but are not limited to, vectors for mammalian host cells including: BPV-1; pHyg; pRSV; pSV2; pTK2 (Maniatis); pIRES (Clontech); pRc/CMV2; pRc/RSV; pSFV1 (Life Technologies); pVPakc Vectors; pCMV vectors; pSG5 vectors (Stratagene); retroviral vectors (e.g., pFB vectors (Stratagene)); pCDNA-3 (Invitrogen) or modified forms thereof; adenoviral vectors; Adeno-associated virus vectors; baculovirus vectors. Other expression vectors for eukaryotic host cells include pESC vectors (Stratagene) for yeast and pFastBac for expression in insect cells (Gibco/BRL, Rockville, Md.).

The expression vectors can include expression control elements for expression in bacterial host cells. These expression control elements can be induced by environmental conditions such as heat-shock, or by addition of agents such as isopropyl-β-D-thiogalactopyranoside (e.g., IPTG) (N Yamaguchi, et al. 2002 The J of Biol Chem 277:6806-6812). Prokaryotic cell expression vectors are well known in the art and are available from several commercial sources. For example, pGEX vector (Promega, Madison, Wis.), pTrcHisB vector (Invitrogen), pET vector (e.g., pET-21, Novagen Corp.), BLUESCRIPT phagemid (Stratagene, LaJolla, Calif.), pSPORT (Gibco BRL, Rockville, Md.), or ptrp-lac hybrids can be used to express the modified hepsin molecules in bacterial host cells.

Host-Vector Systems

The present invention further provides a host-vector system comprising a vector, plasmid, phagemid, BAC, PAC, YAC or cosmid comprising a modified hepsin molecule nucleotide sequence, or a fragment or derivative thereof, introduced into a suitable host cell.

The host-vector system can be used to transcribe and/or express (e.g., produce) the modified hepsin molecules of the invention. A variety of expression vector/host systems can be utilized to carry and express the modified hepsin molecule sequences. The host cell can be either prokaryotic or eukaryotic.

Eukaryotic Host Cells

Methods for Introducing the Modified Hepsin Nucleic Acid Sequence into Cells

Examples of suitable eukaryotic host cells include insect cells, yeast cells, plant cells, or animal cells such as mammalian cells.

An expression system that can be used to express modified hepsin molecule is an insect system. In one such system, Autographa californica nuclear polyhedrosis virus (AcNPV) can be used as a vector to express foreign genes in Spodoptera frugiperda insect cells or in Trichoplusia larvae. The sequence encoding a modified hepsin molecule can be cloned into a nonessential region of the virus, such as the polyhedrin gene, and placed under control of the polyhedrin promoter. Successful insertion of a modified hepsin molecule ucleotide sequence will render the polyhedrin gene inactive and produce recombinant virus lacking coat protein. The recombinant viruses can then used to infect S frugiperda cells or Trichoplusia larvae in which the modified hepsin molecule can be expressed (Smith et al 1983 J Virol 46:584; E K Engelhard, et al, 1994 Proc Nat Acad Sci 91:3224-3227).

In mammalian host cells, a number of viral-based expression systems can be utilized. In cases where an adenovirus is used as an expression vector, a modified hepsin molecule nucleotide sequence can be ligated into an adenovirus transcription/translation vector having the late promoter and tripartite leader sequence. Insertion in a nonessential E1 or E3 region of the viral genome results in a viable virus capable of expressing a modified hepsin molecule in infected host cells (Logan and Shenk 1984 Proc Natl Acad Sci 81:3655-59). In addition, transcription enhancers, such as the rous sarcoma virus (RSV) enhancer, can be used to increase expression in mammalian host cells. In a previous study, baby hamster kidney (BHK) cells were transfected with a plasmid comprising human hepsin cDNA using a calcium phosphate-mediated transfection procedure. The transfected BHK cells expressing human hepsin which activated factor VII (Y Kazama, et al., 1995 J Biol Chem 270:66-72)

In yeast, Saccharomyces cerevisiae, a number of vectors including constitutive or inducible promoters such as beta-factor, alcohol oxidase and PGH can be used. For reviews, see Ausubel et al (Current Protocols in Molecular Biology, John Wiley & Sons, New York N.Y.) and Grant et al (1987 Methods in Enzymology 153:516-544).

In cases where plant expression vectors are used, the expression of a sequence encoding a modified hepsin molecule can be driven by any of a number of promoters. For example, viral promoters such as the 35S and 19S promoters of CaMV (Brisson, et al., 1984 Nature 310:511-514) can be used alone or in combination with the omega leader sequence from TMV (Takamatsu, et al., 1987 EMBO J. 6:307-311). Alternatively, plant promoters such as the small subunit of RUBISCO (Coruzzi et al 1984 EMBO J. 3:1671-1680; Broglie et al 1984 Science 224:838-843); or heat shock promoters (J Winter and R M Sinibaldi 1991 Results Probl Cell Differ 17:85-105) can be used.

In addition, a host cell strain can be chosen for its ability to modulate the expression of the inserted modified hepsin molecule nucleotide sequences or to process the expressed protein in the desired fashion. Such modifications of the expressed modified hepsin molecule include, but are not limited to, acetylation, carboxylation, glycosylation, phosphorylation, lipidation and acylation. Post-translational processing which cleaves a precursor form of the protein (e.g., a prepro protein) can also be important for correct insertion, folding and/or function. Different host cells such as CHO, HeLa, MDCK, 293, W138, etc. have specific cellular machinery and characteristic mechanisms for such post-translational activities and can be chosen to ensure the correct modification and processing of the introduced, foreign protein.

Prokaryotic Host Cells

Examples of suitable prokaryotic host cells include bacteria strains from genera such as Escherichia, Bacillus, Pseudomonas, Streptococcus, and Streptomyces. For example, bacterial cells, such as Epicurian coli XL-1 Blue cells (Stratagene) which have been previously used to produce a naturally-occurring hepsin (Y Kazama, et al., 1995 J Biol Chem 270:66-72) can also be used to produce the modified hepsin molecule.

In bacterial systems, a number of expression vectors can be selected depending upon the use intended for the modified hepsin molecules. For example, when large quantities of the modified hepsin molecules are needed for the induction of antibodies, vectors that direct high level expression of fusion proteins that are soluble and readily purified can be desirable. Such vectors include, but are not limited to, the multifunctional E. coli cloning and expression vectors such as BLUESCRIPT (Stratagene), in which the modified hepsin molecule nucleotide sequence can be ligated into the vector in-frame with sequences for the amino-terminal Met and the subsequent 7 residues of galactosidase so that a hybrid protein is produced. Other vectors include the pIN vectors (Van Heeke & Schuster 1989 J Biol Chem 264:5503-5509), and the like. The pGEX vectors (Promega, Madison Wis.) can also be used to express foreign proteins as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin or factor Xa protease cleavage sites so that the cloned protein of interest can be released from the GST moiety at will.

The methods for introducing the modified hepsin molecule nucleotide sequences into the host cells are well-known methods that depend on the type of vector used and host system employed.

For example, in vertebrate cells, the nucleic acid sequences are introduced with vectors using various methods, including calcium phosphate-mediated DNA transfection (Graham and Van der Eb 1973 Virology 52:456-467; M Wigler, et al 1977 Cell 11:223-232) or other cationic-mediated transfection methods, electroporation (E Neuman, et al 1982 EMBO J. 1:841-845), microinjection (W F Anderson, et al 1980 Proc Natl Acad Sci USA 77:5399-5403; M R Cappechi 1980 Cell 22:479-488; A Graessman, et al 1979 J Virology 32:989-994), or lipid methods including encapsulation of DNA in lipid vesicles (M Schaefer-Ridder 1982 Science 215:166-168). Other methods include the particle gun method. Still other methods include using an adenovirus transcription/translation vector comprising the late promoter and tripartite leader sequence. A nucleic acid sequence can be inserted in a nonessential E1 or E3 region of the adenoviral genome to create a viable virus capable of expressing the protein encoded by the nucleic acid sequence (Logan and Shenk 1984 Proc Natl Acad Sci 81:3655-59). Alternatively, retroviral transfer methods can be used (E Gibloa, et al 1986 BioTechniques 4:504-512).

Plant cells can be introduced by direct DNA transformation or pathogen-mediated transfection. For reviews of such techniques, see Hobbs, S. in: McGraw Yearbook of Science and Technology (1992) McGraw Hill New York N.Y., pp 191-196; or Weissbach and Weissbach (1988) in: Methods for Plant Molecular Biology, Academic Press, New York N.Y., pp 421-463. Alternatively, plant cells can be introduced via a particle-gun method using metal particles.

Prokaryotic host cells are introduced (e.g., transformed) with nucleic acid molecules by electroporation or salt treatment methods (Cohen et al., 1972 Proc Acad Sci USA 69:2110; Maniatis, T., et al., 1989 in: Molecular Cloning, A Laboratory Manual, Cold Spring Harbor Laboratory, Cold Spring Harbor, N.Y.

Selection of Transformed Cells

The cells introduced with the modified hepsin molecule nucleotide sequences can be identified by techniques well known in the art. The cells can be selected, lysed and their DNA content examined for the presence of the introduced sequences using a DNA gel blot method or similar method (Southern 1975 J Mol Biol 98:503; Berent et al., 1985 Biotech 3:208). Alternatively, the proteins produced from the cells of the invention can be assayed via a biochemical assay or immunological method.

Any number of selection systems can be used to recover the introduced (e.g, transformed or transfected) cells. The introduced cells can be selected based on expression of herpes simplex virus thymidine kinase (Wigler, M., et al., 1977 Cell 11:223-32), or adenine phosphoribosyltransferase (Lowy, I. et al., 1980 Cell 22:817-23) genes which can be employed in tk-minus or aprt-minus cells, respectively. Also, antimetabolite, antibiotic or herbicide resistance can be used as a basis for selection; for example, dhfr which confers resistance to methotrexate (Wigler, M., et al., 1980 Proc Natl Acad Sci 77:3567-70); npt, which confers resistance to the aminoglycosides neomycin and G-418 (Colbere-Garapin, F., et al., 1981 J. Mol. Biol. 150:1-14) and als or pat, which confer resistance to chlorsulfuron and phosphinotricin acetyltransferase, respectively. Additional selectable genes have been described, for example, trpB, which allows cells to utilize indole in place of tryptophan, or hisD, which allows cells to utilize histinol in place of histidine (S C Hartman and R C Mulligan 1988 Proc. Natl. Acad. Sci. 85:8047-51). Recently, the use of visible markers has gained popularity with such markers as anthocyanins, beta-glucuronidase and its substrate, GUS, and luciferase and its substrate, luciferin, being widely used not only to identify transformants, but also to quantify the amount of transient or stable protein expression attributable to a specific vector system (C Rhodes, et al., 1995 Methods Mol. Biol. 55:121-131).

Antibodies

The present invention further provides antibodies, such as polyclonal, monoclonal, chimeric, humanized, human, internalizing, neutralizing, anti-idiotypic antibodies, immunologically-active fragments or derivatives thereof, recombinant proteins having immunologically-activity, and immunoconjugates which bind a target polypeptide. The term “target polypeptide” as used herein refers to any of the modified hepsin molecules of the invention, any naturally-occurring hepsin molecule, or fragments or derivatives thereof.

The antibodies of the invention can bind selectively to any of their target polypeptides and will not bind (or will bind weakly) to a non-target polypeptide. The antibodies of the invention can bind to a naturally-occurring target polypeptide or to a recombinant target polypeptides. The antibodies of the invention can bind a target polypeptide expressed by a cell. The target polypeptides expressed by a cell include cell-surface, membrane-bound, or a secreted forms. The antibodies of the invention can bind one or more domains on the target polypeptides, including the cytoplasmic, transmembrane, and/or extracellular domain(s). The antibodies can bind to any of the target polypeptides in their native and/or denatured forms.

The antibodies of the invention can bind a cell or a tissue sample, from a subject, expressing or producing the target polypeptide. Such cells or tissues include prostate, liver, kidney, pancreatic, stomach, thyroid, testicular or ovarian cells or tissues, respectively. The antibodies can bind a cell or tissue sample, from a subject, over-expressing the target polypeptide, including prostate, liver, kidney, pancreatic, stomach, thyroid, testicular or ovarian cells. The antibodies of the invention can bind a cancer cell or tissue sample, from a subject, that is expressing or over-expressing the target polypeptide, including cancer cells from prostate, liver, kidney, pancreas, stomach, thyroid, testes, ovary, or a metastasized cancer cell thereof.

It is understood by those skilled in the art, that the regions or epitopes of the target polypeptide to which an antibody is directed can vary with the intended application. For example, antibodies used for detecting a cell-surface or membrane-bound target polypeptide as expressed on a cell should be directed to an accessible epitope on cell-surface or membrane-bound target polypeptide. Such antibodies can also be useful for detecting a secreted form of the target polypeptide, including target polypeptides that occur in blood serum of a subject. Antibodies that recognize other epitopes, such as the cytoplasmic domain, can be useful for detecting the target polypeptide within a cell.

The antibody of the invention can recognize and bind any portion of the target polypeptide, including the cytoplasmic domain, transmembrane domain, and/or the extracellular domain, or any portion thereof. The target polypeptide is any of the modified hepsin molecules of the invention, or fragments or derivatives thereof.

In one embodiment, the antibody of the invention can recognize and bind the modified hepsin molecule comprising an amino acid sequence beginning with arginine position 1 and ending with leucine at position 376 as shown in FIG. 18, or a fragment or derivative thereof.

In another embodiment, the monoclonal antibodies of the invention are those produced by a hybridoma cell line which is designated 14C7, deposited on Jul. 25, 2002, with the American Type Culture Collection (ATCC), 10801 University Blvd., Manassas, Va. 20110-2209 under the provisions of the Budapest Treaty, and accorded ATCC accession number (PTA-4561). The monoclonal antibody, 14C7, recognizes and binds the modified hepsin molecule of the invention sequences as described in FIG. 18, starting from arginine at position land ending at leucine at position 376, and is an IgG1-kappa isotype.

In another embodiment, the monoclonal antibodies of the invention are those produced by a hybridoma cell line which is designated 94A7, deposited on Sep. 30, 2003, with the American Type Culture Collection (ATCC), 10801 University Blvd., Manassas, Va. 20110-2209 under the provisions of the Budapest Treaty, and accorded ATCC accession number (______). The monoclonal antibody, 94A7, recognizes and binds the hepsin molecule as described in FIG. 17, starting from arginine at position 45 and ending at leucine at position 417, as well as the modified hepsin molecule of the invention sequences as described in FIG. 18, starting from arginine at position 1 and ending at leucine at position 376, and is an IgG2A-kappa isotype.

The present invention provides isolated antibodies. The isolated antibodies are separated from contaminant components that would interfere with bind, detecting, diagnosing, imaging and/or monitoring methodologies. A preferred antibody is purified using any method known in the art. The antibodies can be from any source, including rabbit, sheep, goat, rat, mouse, dog, cat, pig, horse, monkey, ape and human.

In a further embodiment, the antibodies of the invention are made by immunizing a hepsin knock-out animal e.g., a hepsin knock-out mouse (U.S. Pat. No. 5,981,830). In another embodiment, the animal comprises a functional or modified hepsin gene.

Polyclonal Antibodies

The antibodies of the invention can be polyclonal preparations which include a population of different antibodies directed against a different epitope on the immunogen, such as a target polypeptide used as an immunogen.

Polyclonal antibodies can be produced by methods well-known in the art. Polyclonal antibodies can be produced by immunizing animals, usually a mammal, by multiple injections of an immunogen (antigen) and an adjuvant as appropriate (Harlow and Lane, 1988, in: Antibodies: A Laboratory Manual. Cold Spring Harbor Press). The injections can be intradermal, subcutaneous or intraperitoneal. Administration of the immunogen is conducted generally by injection into an animal over a suitable time period and with use of a suitable adjuvant, as is generally understood in the art. During the immunization schedule, titers of antibodies can be taken to determine adequacy of antibody formation. The methods of Dunbar can be used to produce polyclonal antibodies (B S Dunbar and E D Schwoebel 1990 Methods Enzymol 182:663-670).

In general, any antibody (e.g., monoclonal, polyclonal, and the like) can be raised using an isolated target polypeptide, or a fragment as the immunogen. In addition, the immunogen can be a fusion protein including all or a portion of the target polypeptides fused to V5, His, maltose-binding protein, GST, or human Ig. For example, polyclonal antibodies have been previously raised using a fusion protein having the extracellular domain of human hepsin fused to maltose-binding protein (Y Kazama, et al., 1995 J Biol Chem 270:66-72). Cells expressing or overexpressing the target polypeptide can also be used for immunizations. Similarly, any cell engineered to express a target polypeptide can be used.

The full-length target polypeptide can be used as an immunogen to produce the polyclonal antibodies. Alternatively, the amino acid sequence of any of the target polypeptides can be used to select specific regions of these polypeptides for generating antibodies. For example, hydrophobicity and hydrophilicity analyses of these amino acid sequences can be used to identify hydrophilic regions. These amino acid sequences that show immunogenic structure, as well as other regions and domains, can readily be identified using various other methods known in the art (Rost, B., and Sander, C. 1994 Protein 19:55-72), such as Chou-Fasman, Gamier-Robson, Kyte-Doolittle, Eisenberg, Karplus-Schultz or Jameson-Wolf analysis. Fragments including these residues are particularly suited in generating antibodies that bind a modified hepsin molecule. Polyclonal antibodies can be produced using one or more synthetic peptides having the sequence of the cytoplasmic, transmembrane, and/or extracellular domains of the target polypeptide. Polyclonal antibodies have been previously produced using three synthetic peptides having the sequence of the catalytic domain (A Torres-Rosado, et al., 1993 Proc Natl Acad Sci USA 90:7181-7185).

Methods for preparing an immunogen and for preparing immunogenic conjugates of a protein with a carrier such as BSA, KLH, or other carrier proteins are well known in the art.

The animals are typically immunized with about 1 micro gram to about 1 mg immunogen capable of eliciting an immune response, along with an enhancing carrier preparation, such as Freund's complete adjuvant, or an aggregating agent such as alum to produce an immunogen mixture. The immunogen mixture can be injected into the animal at multiple sites. The animals can be boosted with at least one subsequent administration of a lower amount of the immunogen mixture which include about ⅕ to 1/10 the original amount of the immunogen in Freund's complete adjuvant (or other suitable adjuvant). Typically, the animals are bled, the serum is assayed to determine the specific antibody titer, and the animals can be boosted again and assayed until the titer of antibody no longer increases.

In one embodiment, the polyclonal antibodies of the invention are made by immunizing a hepsin knock-out animal e.g., a hepsin knock-out mouse (U.S. Pat. No. 5,981,830). In another embodiment, the animal comprises a functional or modified hepsin gene. The animal can include, but is not limited to any of the following: rabbit, sheep, goat, rat, mouse, dog, cat, pig, horse, monkey, ape or human.

The polyclonal antibody serum can be collected using well known methods or the antibody fraction can be enriched by chromatography with an affinity matrix that selectively binds immunoglobulin molecules such as protein A, to obtain the IgG fraction. The enriched polyclonal antibody can be further enriched using immunoaffinity chromatography such as solid phase-affixed immunogen. For example, the enriched polyclonal antibody fraction is contacted with the solid phase-affixed immunogen for a period of time sufficient for the immunogen to immunoreact with the antibody molecules to form a solid phase-affixed immunocomplex. The bound antibodies are eluted from the solid phase by standard techniques, using of buffers of decreasing pH or increasing ionic strength. The eluted fractions are assayed, and those including the specific antibodies are combined.

Monoclonal Antibodies

The antibodies of the invention can be monoclonal antibodies that bind a specific antigenic site. Examples of the monoclonal antibodies of this invention include, but are not limited to, the antibodies described herein as 47A5, 14C7, 46D12, 38E2, 37G10, 31C1, 11C1, 72H6, 90C1, 85B11, 40F1, 7H3, 27E3, 1A12, and 94A7.

Methods for preparing an immunogen and immunizing an animal are described above. In one embodiment, the monoclonal antibodies of the invention are made by immunizing a hepsin knock-out animal e.g., a hepsin knock-out mouse (U.S. Pat. No. 5,981,830). In another embodiment, the animal comprises a functional or modified hepsin gene. The animal can include, but is not limited to any of the following: rabbit, sheep, goat, rat, mouse, dog, cat, pig, horse, monkey, ape or human.

The monoclonal antibodies can be produced by hybridoma technology first described by Kohler and Milstein (1975 Nature 256:495-497; Brown et al. 1981 J Immunol 127:539-46; Brown et al., 1980 J Biol Chem 255:4980-83; Yeh et al., 1976 Proc Natl Acad Sci USA 76:2927-31; Yeh et al., 1982 Int J Cancer 29:269-75), or human B cell hybridoma techniques (Kozbor et al., 1983 Immunol Today 4:72), or EBV-hybridoma techniques (Cole et al., 1985 Monoclonal Antibodies and Cancer Therapy, Alan R. Liss, Inc., pp. 77-96), or recombinant DNA methods in bacteria, animal cells or plant cells (U.S. Pat. No. 4,816,567), or phage antibody libraries (Clackson, et al., 1991 Nature 352:624-628; Marks, et al., 1991 J Mol Biol 222:581-597). The monoclonal antibodies can be made using a repetitive, multiple site immunization strategy termed RIMMS (K E Kilpatrick, et al., 1997 Hybridoma 16:381-389). An alternative method includes producing affinity matured monoclonal antibodies by fusing a myloma cell line stably transfected with Bcl-2 and immune lymphocytes (K E Kilpatrick, et al., 1997 Hybridoma 16:381-389).

The hybridoma cell secreting the desired antibodies can be screened by immunoassay in which the antigen is the target polypeptide. When the appropriate hybridoma cells secreting the desired antibody are identified, the cells can be cultured either in vitro or by production in ascites fluid. The desired monoclonal antibodies are then recovered from the culture supernatant or from the ascites supernatant.

Chimeric

The chimeric antibodies of the invention comprise an antibody portion (e.g, immunoglobulin portion) from one species or a particular antibody class or subclass, joined to an antibody portion from a different species or antibody class or subclass. The chimeric antibodies can be produced as CDR grafted antibodies of multiple species origin. The portions of the chimeric antibodies can be from any source, including bovine, porcine, murine, equine, canine, feline, monkey, ape, piscine, ovine, avian or human. In particular, the portions of the chimeric antibodies can be from rabbit, sheep, goat, rat, mouse, dog, cat, pig, horse, monkey, ape and human.

For example, one portion of the chimeric antibody can include a constant immunoglobulin portion from one species, and another portion includes a variable region (e.g., antigen combining region). The chimeric antibody comprises a human portion and a non-human portion. The constant region can be derived from human and the variable region can be derived from a non-human species, such as a murine species. The chimeric antibodies can be produced by methods known in the art (Morrison et al., 1985 Proc Natl Acad Sci USA 81:6851; Takeda et al., 1985 Nature 314:452; Cabilly et al., U.S. Pat. No. 4,816,567; Boss et al., U.S. Pat. No. 4,816,397). The chimeric antibody comprises hypervariable loop regions from one species and invariant framework regions from another species. Chimeric antibodies comprising human regions are useful, as they are less likely to be antigenic to a human subject than antibodies with non-human constant regions and variable regions.

The chimeric antibodies of the present invention also comprise antibodies which are chimeric proteins, having several distinct antigen binding specificities (e.g. anti-TNP: Boulianne et al., 1984 Nature 312:643; and anti-tumor antigens: Sahagan et al., 1986 J Immunol 137:1066). The invention also provides chimeric proteins having different effector functions (Neuberger et al., 1984 Nature 312:604), immunoglobulin constant regions from another species and constant regions of another immunoglobulin chain (Sharon et al., 1984 Nature 309:364); Tan et al., 1985 J Immunol 135:3565-3567). Additional procedures for modifying antibody molecules and for producing chimeric antibody molecules using homologous recombination to target gene modification have been described (Fell et al., 1989 Proc Natl Acad Sci USA 86:8507-8511).

In general, the procedures used to produce chimeric antibodies can involve the following steps:

a) identifying and cloning the correct gene segment encoding the antigen binding portion of the antibody molecule; this gene segment (known as the VDJ, variable, diversity and joining regions for heavy chains or VJ, variable, joining regions for light chains or simply as the V or variable region) can be in either the cDNA or genomic form;

b) cloning the gene segments encoding the constant region or desired part thereof;

c) ligating the variable region with the constant region so that the complete chimeric antibody is encoded in a form that can be transcribed and translated;

d) ligating this construct into a vector comprising a selectable marker and gene control regions such as promoters, enhancers and poly(A) addition signals;

e) amplifying this construct in bacteria;

f) introducing this DNA into eukaryotic cells (transfection) most often mammalian lymphocytes;

g) selecting for cells expressing the selectable marker;

h) screening for cells expressing the desired chimeric antibody; and

i) testing the antibody for appropriate binding specificity and effector functions.

Humanized Antibodies

The antibodies of the invention include humanized antibodies, which comprise antibody portions from a human immunoglobulin. In one embodiment, a humanized antibody comprises hypervariable loop regions and/or invariant framework regions from human. In one embodiment, a humanized antibody comprises hypervariable loop regions from non-human species and invariant framework regions from human. A humanized antibody can comprise at least a portion of an immunoglobulin constant region from human. Humanized antibodies can be made according to any known method, including substituting one or more of the non-human antibody CDRs for corresponding human antibody sequences (Teng et al., 1983 Proc Natl Acad Sci USA 80:7308-7312; Kozbor et al., 1983 Immunology Today 4:7279; Olsson et al., 1982 Meth Enzymol 92:3-16; Jones 1986 Nature 321-522-525; Riechmann, et al., 1988 Nature 332:323-329; Verhoeyen et al., 1988 Science 239: 1534-1536; Presta 1992 Curr Op Struct Biol 2:593-596; Carter et al., 1993 Proc Natl Acad Sci USA 89: 4285; Sims et al., 1993 J Immunol 151: 2296).

The present invention also provides antibodies that are more fully-humanized or are fully humanized. These antibodies can be produced using methods known in the art (Vaughan et al., 1998 Nature Biotechnology 16: 535-539; Griffiths and Hoogenboom, “Building an in vitro immune system: human antibodies from phage display libraries”, in: Protein Engineering of Antibody Molecules for Prophylactic and Therapeutic Applications in Man. Clark, M. (Ed.), Nottingham Academic, pp 45-64 (1993); Burton and Barbas, Human Antibodies from Combinatorial Libraries Id., pp 65-82; PCT Patent Application WO98/24893, Jakobovits et al., published Dec. 3, 1997; Jakobovits, 1998, Exp. Opin. Invest. Drugs 7(4): 607-614).

Other methods for producing human antibodies include using the modified hepsin molecule zymogen or modified hepsin protease, or a fragment or derivative thereof, as an antigen to sensitize human lymphocytes to the antigen in vitro, followed by EBV-transformation or hybridization of the antigen-sensitized lymphocytes with mouse or human lymphocytes (Borrebaeck et al., 1988 Proc Natl Acad Sci USA 85:3995-99).

Alternatively, human antibodies can be produced using transgenic animals such as mice which are incapable of expressing endogenous immunoglobulin heavy and light chain genes, but which can express human heavy and light chain genes. The transgenic mice are immunized in the normal fashion with a selected antigen, such as the modified hepsin molecule, or a fragment or derivative thereof. The human immunoglobulin transgenes harbored by the transgenic mice rearrange during B cell differentiation, and subsequently undergo class switching and somatic mutations. Thus, using this technology, it is possible to produce therapeutically useful IgG, IgA, and IgB antibodies. For an overview of this technology to produce human antibodies, see Lonberg and Haszar (1995 Int Rev Immunol 13:65-93). A detailed discussion of this technology for producing human antibodies and human monoclonal antibodies can be found in U.S. Pat. Nos. 5,625,126; 5,633,425; 5,569,825; 5,661,016; and 5,545,806.

Internalizing Antibodies

The antibodies of the invention can be internalizing antibodies which enter (e.g., internalize) a cell upon bind to the target polypeptide on the cell. An internalizing antibody that enters into a cell can inhibit growth of the cell or kill the cell. Thus, internalizing antibodies are useful for therapeutic methods such as inhibiting cell growth and/or inducing cell death. The internalization of the antibody can be analyzed using I¹²⁵ labeled antibodies (Wolff et al., 1993 Cancer Res. 53: 2560-2565).

The internalizing antibodies of the invention exhibit a rate of entering the cell. The rate can be measured starting from the time the cell is contacted with the internalizing antibody, or starting from the time a subject is administered the internalizing antibody. In one embodiment, the internalizing antibodies exhibit a rate of entering the cell within about 24 hours, or within about 12 hours, or within about 1 hour. A preferred internalizing antibody enters a cell, after contacting the cell, within about 30 to 60 minutes, or more preferably in less than about 30 minutes. In these embodiment, the rate of internalizing can be measured from the time the cell is contacted with the internalizing antibody, or from the time a subject is administered the internalizing antibody.

Neutralizing Antibodies

The invention provides neutalizing antibodies, or fragments or derivatives thereof, to target specific antigens. Administration of neutralizing antibodies, or fragments or derivatives thereof, to a substrate or sample having the target antigen can render the target antigen ineffective in its actions, processes and/or potentials. Neutralizing antibodies, or fragments or derivatives thereof, can render ineffective molecules, actions, processes and/or potentials associated with the target antigen. Neutralizing antibodies, or fragments or derivatives thereof, can inhibit cellular actions, processes and/or potentials, such as cell cycling, cell differentiation, cell growth. In one embodiment of the invention, the neutralizing antibodies inhibit the cleavage and/or activation of hepsin molecules (see Example 8). In another embodiment, the neutralizing antibodies of the invention inhibit activated hepsin from recognizing, binding and/or cleaving its substrate. Examples of the neutralizing antibodies of this invention include but are not limited to the monoclonal antibodies described herein as 1A12 and 94A7.

Recombinant Protein

Further, the invention provides recombinant proteins which exhibit the functional activity of an antibody of the invention (e.g, bind a target polypeptide such as wildtype or modified hepsin molecule, or fragments or derivatives thereof). The recombinant proteins of the invention can be produced by a cell engineered to express the recombinant protein. The recombinant protean be produced by methods used to produce conventional antibodies, such as polyclonal technology, hybridoma technology, and/or phage library technologies (RD Mayforth and J Quintans 1990 New Eng J Med 323:173-178; T A Waldmann 1991 Science 252:1657-1662; G Winter and C Milstein 1991 Nature 349:293-299; SL Morrison 1992 Ann Rev Immunol 10:239-266).

The recombinant proteins of the invention can be a single chain polypeptide molecule that bind the target polypeptides. The heavy (H) and light (L) chains of an Fv portion of an antibody can be encoded by a single nucleotide sequence and include a linker sequence (Bird et al. 1988 Science 242:423-426; Huston et al. 1988 Proc Natl Acad Sci USA 85:5879-5883).

The recombinant proteins can be mono-specific or bispecific. The bi-specific proteins will have one portion that binds the target polypeptide and another portion will bind a different target polypeptide. The mono-specific proteins have one portion that binds the target polypeptide.

Antibodies that Competitively Inhibits

The invention provides antibodies which competitively inhibit the immunospecific binding of any of the antibodies of the invention to the target polypeptide. The competitive inhibiting antibody can bind to the same epitope as the epitope bound by the antibodies of the invention. These antibodies can be identified by routine competition assays using, for example, any of the antibodies of the invention (Harlow, E. and Lane, D. 1988 Antibodies, A Laboratory Manual, Cold Spring Harbor Laboratory, Cold Spring Harbor, N.Y.).

As an example, the competition assays can be a competitive ELISA assay. The competitive ELISA assay can include coating the wells of a microtiter plate with a target polypeptide (e.g, a wiletype or modified hepsin, or fragments or derivatives thereof), an optional step includes pre-incubating with a candidate antibody, contacting the microtiter plate with a labeled antibody of the invention. The labeled antibody can be, for example, an antibody of the invention labeled with a detectable and/or measurable label, such as biotin. The amount of labeled antibody of the invention which is bound to the target polypeptide is indirectly correlated with the ability of the candidate antibody to compete for binding to the same epitope (e.g., to block the labeled antibody of the invention from binding the same epitope). The amount of bound labeled antibody of the invention can be measured. The candidate antibody is considered to be a competitive inhibiting antibody if it can block binding of at least about 20%, or at least about 20 to 50%, or at least 50% or more of the labeled antibody of the invention. It is appreciated by those in the art that other competition assays can be performed.

Anti-Idiotypic Antibodies

The present invention provides anti-idiotypic antibodies that mimic the target polypeptides. The anti-idiotypic antibodies bind an idiotype on any of the antibodies of the invention.

Methods for producing anti-idiotypic antibodies are well known in the art (Wagner et al., 1997 Hybridoma 16: 33-40; Foon et al., 1995 J Clin Invest 96: 334-342; Herlyn et al., 1996, Cancer Immunol Immunother 43: 65-76). Such anti-idiotypic antibodies can be used in anti-idiotypic therapy as presently practiced with other anti-idiotypic antibodies directed against tumor antigens.

Antibody Fragments

The invention also encompasses antibody fragments that recognize and bind a target polypeptide. Use of immunologically reactive fragments, such as the Fab, Fab′, or F(ab′)₂ fragments is often preferable, especially in a therapeutic context, as these fragments are generally less immunogenic than the whole immunoglobulin. An antibody fragment comprises a portion of an intact antibody, such as, for example, the antigen-binding or variable region of the intact antibody. The antibody fragment can comprise the constant region of the intact antibody. Antibody fragments can include Fab, F(ab′)₂, or Fv fragments (U.S. Pat. No. 5,641,870; Zapata, et al. 1995 Protein Eng 8:1057-1062), also single-chain antibodies and recombinant proteins which bind the target polypeptides. The antibody fragments can be generated by papain digestion of intact antibodies to produce Fab and Fc fragments, or by pepsin digestion to produce F(ab′)2 fragments.

Further, antibody effector functions can be modified so as to enhance the therapeutic effect of the antibody on cancers. For example, cysteine residues can be engineered into the Fc region, permitting the formation of interchain disulfide bonds and the generation of homodimers which can have enhanced capacities for internalization, ADCC and/or complement-mediated cell killing (Caron et al., 1992 J Exp Med 176: 1191-1195; Shopes, 1992, J. Immunol. 148: 2918-2922). Homodimeric antibodies can also be generated by cross-linking techniques known in the art (Wolff et al., 1993 Cancer Res. 53: 2560-2565).

Labeled Antibodies

The present invention provides antibodies, such as polyclonal, monoclonal, chimeric, humanized, human, internalizing, neutralizing, anti-idiotypic antibodies, immunologically-active fragments thereof, recombinant proteins having immunologically-activity, and immunoconjugates, which are labeled with a detectable marker. The detectable markers include, but are not limited to, a radioisotope, a fluorescent compound, a bioluminescent compound, chemiluminescent compound, a chromophore, a metal chelator, biotin, or an enzyme.

The labeled antibodies of the invention can be particularly useful in various immunological assays for detecting the target polypeptides in a biological sample and/or in diagnostic imaging methodologies. Such assays generally comprise one or more labeled antibodies that recognize and bind the target polypeptides, and include various immunological assay formats well known in the art, including but not limited to various types of precipitation, agglutination, complement fixation, competition, inhibition, radioimmunoassays (RIA), enzyme-linked immunosorbent assays (ELISA), enzyme-linked immunofluorescent assays (ELIFA) (H Liu et al. 1998 Cancer Research 58: 4055-4060), immunohistochemical analyses and the like.

In addition, immunological imaging methods that detect cells expressing the target polypeptides are also provided, including but not limited to radioscintigraphic imaging methods using the labeled antibodies of the invention. Such assays can be clinically useful in the detection and monitoring the number and/or location of cells expressing the target polypeptides.

Conjugated Antibodies

The antibodies of the invention, such as polyclonal, monoclonal, chimeric, human, humanized, internalizing, neutralizing, anti-idiotypic antibodies, immunologically-active fragments thereof, recombinant proteins having immunologically-activity or fragment thereof can be conjugated to therapeutic agent, such as a cytotoxic agent, thereby resulting in an immunoconjugate. For example, the therapeutic agent includes, but is not limited to, an anti-tumor drug, a toxin, a radioactive agent, a cytokine, a lymphokine, oncostatin, a second antibody or an enzyme. Further, the invention provides an embodiment wherein the antibody of the invention is linked to an enzyme that converts a prodrug into a cytotoxic drug.

Examples of cytotoxic agents include, but are not limited to ricin, ricin A-chain, doxorubicin, daunorubicin, taxol, ethiduim bromide, mitomycin, etoposide, tenoposide, vincristine, vinblastine, colchicine, dihydroxy anthracin dione, actinomycin D, diphteria toxin, epithilones, Pseudomonas exotoxin (PE) A, PE40, abrin, arbrin A chain, modeccin A chain, alpha-sarcin, gelonin, mitogellin, retstrictocin, phenomycin, enomycin, curicin, crotin, calicheamicin, sapaonaria officinalis inhibitor, maytansinoids, and glucocorticoid and other chemotherapeutic agents, as well as radioisotopes. Suitable radioisotopes include the following: Antimony-124, Antimony-125, Arsenic-74, Barium-103, Barium-140, Beryllium-7, Bismuth-j206, Bismuth-207, Cadmium-109, Cadmium-115m, Calcium-45, Cerium-139, Cerium-141, Cerium-144, Cesium-137, Chromium-51, Cobalt-56, Cobalt-57, Cobalt-58, Cobalt-60, Cobalt-64, Erbium-169, Europium-152, Gadolinium-153, Gold-195, Gold-199, Hafnium-175, Hafnium-181, Indium-111, Iodine-123, Iodine-131, Iridium-192, Iron-55, Iron-59, Krypton-85, Lead-210, Lutetium-177, Manganese-54, Mercury-197, Mercury-203, Molybdenum-99, Neodymium-147, Neptunium-237, Nickel-63, Niobium-95, Osmium-185+191, Palladium-103, Platinum-195m, Praseodymium-143, Promethium-147, Protactinium-233, Radium-2226, Rhenium-186, Rubidium-86, Ruthenium-103, Ruthenium-106, Scandium-44, Scandium-46, Selenium-75, Silver-110m, Silver-11, Sodium-22, Strontium-85, Strontium-89, Strontium-90, Sulfur-35, Tantalum-182, Technetium-99m, Tellurium-125, Tellurium-132, Thallium-170, Thallium-204, Thorium-228, Thorium-232, Tin-113, Titanium-44, Tungsten-185, Vanadium-48, Vanadium-49, Ytterbium-169, Yttrium-88, Yttrium-90, Zinc-65, and Zirconium-95. Antibodies can also be conjugated to an anti-cancer pro-drug activating enzyme capable of converting the pro-drug to its active form.

Techniques for conjugating or joining therapeutic agents to antibodies are well known (Arnon et al., “Monoclonal Antibodies For Immunotargeting Of Drugs In Cancer Therapy”, in: Monoclonal Antibodies And Cancer Therapy, Reisfeld et al. (eds), pp 243-56 (Alan R Liss, Inc 1985); Hellstrom et al., “Antibodies For Drug Delivery”, in: Controlled Drug Delivery (2nd Ed), Robinson et al. (eds), pp 623-53 (Marcel Dekker, Inc. 1987); Thorpe, “Antibody Carriers Of Cytotoxic Agents In Cancer Therapy: A Review”, in: Monoclonal Antibodies '84: Biological And Clinical Applications, Pinchera et al. (eds), pp. 475-506 (1985); and Thorpe et al., “The Preparation And Cytotoxic Properties Of Antibody-Toxin Conjugates”, Immune. Rev, 62:119-58 (1982); Sodee et al., 1997, Clin Nuc Med 21: 759-766). In some circumstances, direct conjugation using, for example, carbodiimide reagents can be used; in other instances linking reagents such as those supplied by Pierce Chemical Co., Rockford, Ill., can be effective.

Radiolabeling of antibodies is accomplished using a chelating agent which is covalently attached to the antibody, with the radionuclide inserted into the chelating agent. Preferred chelating agents are set forth in Srivagtava et al. Nucl. Med. Bio. 18:589-603, 1991 and McMurry et al., J. Med. Chem. 41:3546-3549, 1998. or derived from the so-called NOTA chelate published in H. Chong, K. et al., J. Med. Chem. 45:3458-3464, 2002, all of which are incorporated herein in full by reference. Particularly preferred for conjugation of radioisotopes to an RG1 antibody are derivatives of the bifunctional chelator p-SCN-Benzyl-DPTA (Brechbiel et al. Inorg. Chem. 25:2772-2781, 1986); for example, cyclohexyl-DTPA (CHX-A″-DTPA, Wu et al., Bioorg. Med. Chem. 10:1925-1934, 1997) and MX-DTPA (1B4M-DTPA, McMurry et al., J. Med. Chem., 41: 3546-3549, 1998), as well as 1,4,7-triazacyclononane-N,N′,N″-triacetic acid (NOTA) (Chong et al. J. Med. Chem. 45:3458-3464, 2002). Conjugation can be accomplished by the method of Nikula et al. Nucl. Med. Biol. 3:387-390, 1995. Particularly preferred for use as a detectable marker for immunoscintigraphy are the radioisotopes ¹¹¹In or ^{99m}Tc. Preferred detectable markers for positron emitting tomography are ⁴³Sc, ⁴⁴Sc, ⁵²Fe, ⁵⁵Co, ⁶⁸Ga, ⁶⁴Cu, ⁸⁶Y and ^{94m}Tc. For immunotherapy, the beta-emitting radioisotopes ⁴⁶Sc, ⁴⁷Sc, ⁴⁸Sc, ⁷²Ga, ⁷³Ga, ⁹⁰Y, ⁶⁷Cu, ¹⁰⁹Pd, ¹¹¹Ag, ¹⁴⁹Pm, ¹⁵³Sm, ¹⁶⁶Ho, ¹⁷⁷Lu, ¹⁸⁶Re, and ¹⁸⁸Re and the alpha-emitting isotopes ²¹¹At, ²¹¹Bi, ²¹²Bi, ²¹³Bi and ²¹⁴Bi, can be used. Preferred are ⁹⁰Y, ¹⁷⁷Lu, ⁷²Ga, ¹⁵³Sm, ⁶⁷Cu and ²¹²Bi, and particularly preferred are ⁹⁰Y and ¹⁷⁷Lu.

The immunoconjugate can be used for targeting the therapeutic agent to a cell expressing the target polypeptides (ES Vitetta, et al., 1993 Immunotoxin Therapy, in: DeVita, Jr., V. T. et al., eds, Cancer: Principles and Practice of Oncology, 4th ed., JB Lippincott Co., Philadelphia, 2624-2636).

Pharmaceutical Compositions and Kits

The present invention provides pharmaceutical compositions comprising the molecules of the invention admixed with an acceptable carrier or adjuvant which is known to those of skill of the art. The pharmaceutical compositions preferably include suitable carriers and adjuvants which include any material which when combined with a molecule of the invention retains the molecule's activity and is non-reactive with the subject's immune system. These carriers and adjuvants include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, phosphate buffered saline solution, water, emulsions (e.g. oil/water emulsion), salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances and polyethylene glycol. Other carriers can also include sterile solutions; tablets, including coated tablets and capsules. Typically such carriers include excipients such as starch, milk, sugar (e.g. sucrose, glucose, maltose), certain types of clay, gelatin, stearic acid or salts thereof, magnesium or calcium stearate, talc, vegetable fats or oils, gums, glycols, or other known excipients. Such carriers can also include flavor and color additives or other ingredients. Compositions comprising such carriers are formulated by well-known conventional methods. Such compositions can also be formulated within various lipid compositions, such as, for example, liposomes as well as in various polymeric compositions, such as polymer microspheres.

Further provided are kits comprising compositions of the invention, in free form or in pharmaceutically acceptable form. The kit can comprise instructions for its administration. The kits of the invention can be used in any method of the present invention.

Crystal Structure of Modified Hepsin Molecules

The present invention provides crystals and/or molecular structures of the modified hepsin molecules of the invention. Modified hepsin molecules are expressed from the recombinant plasmids described herein e.g., SEQ ID NOs: 9-11. The expressed protein can be crystallized according to protocols and conditions known to those skilled in the art (A. McPherson, 1999, Crystallization of Biological Macromolecules, Cold Spring Harbor Laboratory Press, Cold Spring Harbor, ISBN: 0879696176; A. Ducruix and R. Giege, 1999, Crystallization of Nucleic acids and Proteins: A Practical Approach, 2^{nd} Edition, Oxford University Press; ISBN: 0199636788)

Once the modified hepsin crystals are formed, the crystals can be analyzed by X-ray crystallography according to protocols and conditions well known in the art (J Drenth, 1999, Principles of Protein X-ray Crystallography, 2^{nd} Edition, Springer Verlag; ISBN: 0387985875). Diffraction data gathered by X-ray crystallography can be analyzed and used to construct a structure of the modified hepsin molecule (G. Rhodes, 2000, Crystallography Made Crystal Clear, 2^{nd} Edition, Academic Press; ISBN: 0125870728; A. Leach, 2001, Molecular Modelling: Principles and Applications, 2^{nd} Edition, Prentice Hall; ISBN: 0582382106). The crystals and information obtained from the crystals can be used to screen for, or design, hepsin ligands as described infra. Additionally, the molecular structure of hepsin can be used to design hepsin derivatives as described infra.

Methods

The present invention also comprises methods using the compositions described supra.

Detecting Modified Hepsin Molecules

One skilled in the art can readily assay for the presence of a modified hepsin molecule by performing enzyme assays known in the art. In one embodiment, an enzyme assay comprises the following steps: 1) contacting the modified hepsin molecule, in the zymogen form, with an activating protease that recognizes and cleaves the substitute activation sequence to produce an activated modified hepsin molecule; 2) contacting the activated modified hepsin molecule with a oligo-peptide substrate to produce a cleaved substrate; and 3) detecting the presence of the cleaved substrate. The oligo-peptide substrate can be linked to a fluorescent agent that permits detection of the cleaved substrates via fluorescent emission (N Yamaguchi, et al., 2002 The J Biol Chem 277:6806-6812).

Methods for detecting the presence of a functionally active, modified hepsin molecule include detecting cleavage of gelatin or casein in a zymography gel procedure (Lin et al., 1997, JBC 272:9147-52; N Yamaguchi, et al., 2002 The J Biol Chem 277:6806-6812).

Other methods for detecting the presence of a functionally active modified hepsin molecule can be performed using the methods described in the Examples section, herein.

Any of these methods can be performed in the presence of an active site inhibitor such as p-amidinophenylmethanesulfonyl fluoride hydrocholoride, leupeptin, or antipain (J Kraut, 1977, Ann Rev Biochem 46:331-358). Other inhibitors include agents that competitively inhibit a cognate protease from recognizing and/or cleaving the substitute activation sequence in the modified hepsin molecule. Inhibitors can also include antibodies that recognize and bind the substitute activation sequence.

Detecting Hepsin Ligands

Another aspect of the invention relates to screening methods for identifying hepsin ligands i.e., agents of interest and/or cellular constituents that bind to, or are bound by, hepsin molecules and/or modulate the biological activity of hepsin molecules. One goal of such screening methods is to identify hepsin ligands that can cause a change in the biological activity of hepsin, such as activation or inhibition, thereby decreasing diseases associated with abnormal cellular expression of the ligand and/or hepsin. Another goal is to identify hepsin ligands that can be affected e.g., activation or inhibition, by hepsin modulation thereby decreasing diseases associated with abnormal cellular expression of the ligand and/or hepsin

The present invention provides modified hepsin molecules, having the same or similar functional activity of wild-type hepsin molecules, which are inhibited by compounds known to inhibit the activity of wild-type hepsin molecules. The modified hepsin molecules of the invention can be used in screening assays to identify ligands that interact with, compete with, and/or regulate, hepsin e.g., inhibitors of hepsin.

In one embodiment, a screening assay comprises the following: contacting labeled modified hepsin molecule with a test agent or cellular extract, under conditions that allow association (e.g., binding) of the modified hepsin molecule with the test agent or a component of the cellular extract; and determining if a complex comprising the agent or component associated with the modified hepsin molecule is formed. The screening methods are suitable for use in high throughput screening methods.

The binding of an agent with a modified hepsin molecule can be assayed using a shift in the molecular weight or a change in biological activity of the unbound modified hepsin molecule, or the expression of a reporter gene in a two-hybrid system (Fields, S. and Song, O., 1989, Nature 340:245-246). The method used to identify whether a agent/cellular component binds to a modified hepsin molecule will be based primarily on the nature of the modified hepsin molecule used. For example, a gel retardation assay can be used to determine whether an agent binds to a modified hepsin molecule, or a fragment or derivative thereof. Alternatively, immunodetection and biochip (e.g., U.S. Pat. No. 4,777,019) technologies can be adopted for use with the modified hepsin molecule. Another method for identifying agents that bind with a modified hepsin molecule employs TLC overlay assays using glycolipid extracts from immune-type cells (K. M. Abdullah, et al., 1992 Infect. Immunol. 60:56-62). A skilled artisan can readily employ numerous art-known techniques for determining whether a particular agent binds to a modified hepsin molecule of the invention.

In one embodiment, hepsin ligands can be detected by binding a labelled modified hepsin molecule of the invention. Labels can include those well known in the art such as radio-isotope labels, fluorescent labels and others. The hepsin ligands so detected can be soluble or attached to a cell or tissue sample from a subject. The hepsin ligands can also be present or overexpressed in tumor cells or tumor tissues from a subject.

In another embodiment, the biological activity of a modified hepsin molecule, as part of the complex, can be analyzed as a means for identifying agonists and antagonists of hepsin activity.

For example, a method used to isolate cellular components that bind CD22 (D. Sgroi, et al., 1993 J. Biol. Chem. 268:7011-7018; L. D. Powell, et al., 1993 J. Biol. Chem. 268:7019-7027) can be adapted to isolate cell-surface glycoproteins that bind to modified hepsin molecule by contacting cell extracts with an affinity column having immobilized anti-modified hepsin antibodies.

In another example, chromogenic and/or fluorogenic, substrate-based assays such as those described by Lottenberg et al. (Lottenberg, R., Christensen, U., Jackson, C., and Coleman, P. L., 1981, Methods Enzymol, Assay of coagulation proteases using peptide chromogenic and fluorogenic substrates, 80:341-361) or Phillips et al (Phillips, G., Davey, D. D., Eagen, K. A., Koovakkat, S. K., Liang, A., Ng, H. P., Pinkerton, M., Trinh, L., Whitlow, M., Beatty, A. M., and Morrissey, M. M., 1999, J Med Chem, Design, synthesis, and the activity of 2,6-diphenoxypyridine-derived factor Xa inhibitors, 42(10):1740-56) can be performed to detect the presence of activated, modified hepsin protease. Alternatively the same assays can be used for identifying a compound of interest from a library of candidate compounds, where the compound of interest inhibits the activity of the modified hepsin protease.

Hepsin inhibitory compounds that can be used in screening assays include 4-amidiophenylmethylsulfonyl fluoride, aprotinin, antithrombin III (Kazama et al, J. Biol. Chem., 1995, 270:66-72), leupeptin, antipain, Nα-tosyl-L-lysin chloromethyl ketone and soybean trypsin inhibitor (Zhukov et al., Biochim Biophys Acta, 1997, 1337:85-95).

Another embodiment of the assay includes screening agents and cellular constituents that bind to modified hepsin molecules using a yeast two-hybrid system (Fields, S. and Song, O., supra) or using a binding-capture assay (Harlow, supra). Generally, the yeast two-hybrid system is performed in a yeast host cell carrying a reporter gene, and is based on the modular nature of the GAL transcription factor that has a DNA binding domain and a transcriptional activation domain. The two-hybrid system relies on the physical interaction between a recombinant protein that comprises the DNA binding domain and another recombinant protein that comprises the transcriptional activation domain to reconstitute the transcriptional activity of the modular transcription factor, thereby causing expression of the reporter gene. Either of the recombinant proteins used in the two-hybrid system can be constructed to include the modified hepsin—encoding sequence to screen for hepsin binding partners. The yeast two-hybrid system can be used to screen cDNA expression libraries (G. J. Hannon, et al. 1993 Genes and Dev. 7: 2378-2391), random aptmer libraries (J. P. Manfredi, et al. 1996 Molec. And Cell. Biol. 16: 4700-4709) or semi-random (M. Yang, et al. 1995 Nucleic Acids Res. 23: 1152-1156) aptmer libraries for hepsin ligands.

Another embodiment of the invention involves using the crystal structure of the modified hepsin molecules of the invention to screen for hepsin ligands and/or aid in the design i.e., to rationally design, such ligands.

For example, in order to screen for agents that bind hepsin, X-ray crystallography can be employed using the modified hepsin molecules of the invention. The modified hepsin molecule can be crystallized and potential binding agents can be exposed to the crystal. Alternatively, the modified hepsin molecule of the invention can be exposed to a potential binding agent in solution and the modified hepsin moleucule can be co-crystallized with the binding agent. X-ray diffraction datais then obtained and the crystal structure solved in order to determine whether the agent binds to the modified hepsin to form a ligand/receptor complex.

Additionally, X-ray crystallography can be used to aid in the design of hepsin ligands or to modify existing ligands. Modified hepsin molecules can be crystallized and diffraction data obtained using standard X-ray crystallography. This X-ray crystallography data provides a molecular structure of the modified hepsin molecule. Once the molecular structure is known, the structure can be used to design hepsin ligands with varying properties, with each property specially designed for the future function of the ligand. Alternatively, with the hepsin structural data, known hepsin ligands can be modified to add desired traits. For example, the hepsin ligand can be modified in its binding specificity, affinity, biological activity and/or safety profile.

Modified hepsin molecules used in screening assays can include, but are not limited to: an isolated modified hepsin molecule, or a fragment or derivative thereof; nucleotide sequences encoding modified hepsin molecules, or a fragment or derivative thereof, a cell that has been altered to express a modified hepsin molecule, or a fragment or derivative thereof; a fraction of a cell that has been altered to express a modified hepsin molecule, or a fragment or derivative thereof, hepsin antibodies e.g., anti-idiotypic antibodies.

The candidate agents to be tested for binding with modified hepsin molecule and/or modulating the activity of modified hepsin molecule can be, as examples, peptides, small molecules, and vitamin derivatives, as well as carbohydrates. A skilled artisan can readily recognize that there is no limit as to the structural nature of the agents tested for binding to modified hepsin molecules. One class of agents is peptide agents whose amino acid sequences are chosen based on the amino acid sequence of the modified hepsin molecule. Small peptide agents can serve as competitive inhibitors of modified hepsin molecules.

Candidate agents that are tested for binding with modified hepsin molecules and/or modulating the activity of modified hepsin molecules can be randomly selected or rationally selected.

As used herein, an agent is randomly selected when the agent is chosen randomly without considering the specific sequences of the modified hepsin molecule. Examples of randomly selected agents are members of a chemical library, a peptide combinatorial library, a growth broth of an organism, or plant extract.

As used herein, an agent is rationally selected when the agent is chosen on a nonrandom basis that is based on the sequence of the target site and/or its conformation in connection with the agent's action. Agents can be rationally selected by utilizing the peptide sequences that make up the modified hepsin molecule. For example, a rationally selected peptide agent can be a peptide whose amino acid sequence is identical to a selected fragment of a modified hepsin molecule.

The cellular extracts to be tested for binding with modified hepsin molecules and/or modulating the activity of modified hepsin molecules can be, as examples, aqueous extracts of cells or tissues, organic extracts of cells or tissues or partially purified cellular fractions. A skilled artisan can readily recognize that there is no limit as to the source of the cellular extracts used in the screening methods of the present invention.

Designing Hepsin Derivatives

In another embodiment of the invention, the molecular structure of hepsin, derived from X-ray crystallography data, can be used to design hepsin derivatives. The modified hepsin molecules of the invention can be crystallized and diffraction data obtained using standard X-ray crystallography techniques. The structure of the hepsin molecule can be determined and a model formed. The properties of the hepsin molecule can be modified to form a hepsin derivative based on the hepsin molecular structure. In one example, introducing a mutation into a hepsin molecule can make a hepsin derivative. The hepsin derivatives so modified can have altered properties such as altered binding specificity, affinity, biological activity and/or safety profile.

Additionally, once hepsin derivatives are made, ligands and/or antibodies to the hepsin derivatives can be produced.

Uses of the Antibodies of the Invention

The reactivity of the antibodies of the invention against the target polypeptides can be established by a number of well known methods, including Western blot, immunoprecipitation, ELISA, and FACS analyses using, as appropriate, any one of the various target polypeptides, or cells expressing any one of the various target polypeptides, or extracts thereof. The various target polypeptides, include naturally-occurring hepsin molecules or any of the modified hepsin molecules of the invention. The antibodies can be characterized in various in vitro assays, including complement-mediated tumor cell lysis, antibody-dependent cell cytotoxicity (ADCC), antibody-dependent macrophage-mediated cytotoxicity (ADMMC), tumor cell proliferation, and the like.

The antibodies of the invention can be used in methods for detecting the presence of any one of the target polypeptides in a sample. The sample includes tissue and biological fluids including, but is not limited to, tissue extracts, urine, blood, serum, phlegm, and sputum. For example, immunofluorescent methods have been previously used to detect hepsin in prepared tissue sections using anti-hepsin, polyclonal antibodies (A Torres-Rosado, et al., 1993 Proc Natl Acad Sci USA 90:7181-7185).

The antibodies of the invention can also be useful for diagnosing, imaging and/or monitoring a cancer cell or a metastasized cancer cell expressing or over-expressing the target polypeptide. The methods for detecting include detecting the presence of a target polypeptide on a cell or in a tissue sample or a prepared tissue sample slice from a subject. These methods can include various immunological assay formats well known in the art, including but not limited to various types of precipitation, agglutination, complement fixation, radioimmunoassays (RIA), enzyme-linked immunosorbent assays (ELISA), enzyme-linked immunofluorescent assays (ELIFA) (H Liu et al. 1998 Cancer Research 58: 4055-4060), immunohistochemical analysis and the like. In addition, immunological imaging methods capable of detecting a cancer are also provided by the invention, including but limited to radioscintigraphic imaging methods using labeled antibodies of the invention. The imaging methods include immunoscintigraphy using Indium-11, or other isotope, such as the method used for detecting detect recurrent and metastatic prostate carcinomas (Sodee et al., 1997 Clin Nuc Med 21: 759-766).

The antibodies of the invention can be used to detect a cell expressing or producing the target polypeptide. Such cells include prostate, liver, kidney, pancreatic, stomach, thyroid, testicular or ovarian cells. The antibodies can bind a cell over-expressing the target polypeptide, including prostate, liver, kidney, pancreatic, stomach, thyroid, testicular or ovarian cells.

The antibodies of the invention can be used to detect the target polypeptide in a tissue sample expressing the target polypeptide in a subject. The antibodies can be used to detect over-expression of the target polypeptide in tissue samples. Such tissue samples include samples from prostate, liver, kidney, pancreas, stomach, thyroid, testes or ovary.

The antibodies of the invention can detect cancer cell that is expressing or over-expressing the target polypeptide, including cancer cells from prostate, liver, kidney, pancreas, stomach, thyroid, testes, ovary, or a metastasized cancer cell thereof.

The antibodies is also be useful in methods for treating a cancer, where the antibodies inhibit the growth or kill a cancer cell expressing or over-expressing the target polypeptide. It has been previously shown in an in vitro procedure that affinity-purified, anti-human hepsin polyclonal antibodies inhibit the growth of hepatoma cells that express hepsin (e.g., HepG2 cells) (A Torres-Rosado, et al., 1993 Proc Natl Acad Sci USA 90:7181-7185).

The antibodies is also be used in methods for purifying various target polypeptides, including naturally-occurring hepsin molecules or any of the modified hepsin molecules of the invention. One method for purifying a target polypeptide comprises incubating an antibody of the invention, which has been coupled to a solid matrix, with a lysate or other solution having the target polypeptides under conditions which permit the antibody to bind to the target polypeptide; washing the solid matrix to eliminate impurities; and eluting the target polypeptide from the coupled antibody.

The antibodies of the invention can be used to isolate or enrich for cells expressing any one of the target polypeptides (e.g., a hepsin-positive cell) using antibody-based cell sorting and/or affinity purification techniques. The presence of any one of the target polypeptides on a tumor cell (alone or in combination with other cell surface markers) can be used to distinguish and/or isolate tumor cells from other cells. The cells expressing or over-expressing a target polypeptide includes normal cells and cancer cells from prostate, liver, kidney, pancreas, stomach, thyroid, testes, ovary, or metastasized tumor cells thereof.

The cells expressing any one of the target polypeptides, which are isolated or enriched using an antibody of the invention, can be grown in culture or as xenograft tumors in animal models (e.g., SCID or other immune deficient mice) thereby permitting the evaluation of various transgenes or candidate therapeutic compounds on the growth or other phenotypic characteristics of a relatively homogeneous population of cells. These isolated or enriched cells can also be used for isolating preparations of nucleic acid molecules encoding gene products that have biological relevance to cancer disease progression, including cancer of the prostate, liver, kidney, pancreas, stomach, thyroid, testes, ovary, or metastasized tumors thereof.

The antibodies of the invention can be used for isolating or enriching tumor cells expressing any one of the various target polypeptides, in order to expand the number of cells from a subject having cancer. In this way, for example, a limited biopsy sample from a subject having cancer can be expanded and tested for the presence of diagnostic and prognostic genes, proteins, chromosomal aberrations, gene expression profiles, or other relevant genotypic and phenotypic characteristics, without the potentially confounding variable of contaminating cells. In addition, such cells can be evaluated for neoplastic aggressiveness and metastatic potential in animal models. Similarly, patient-specific cancer vaccines and cellular immunotherapeutics can be created from such cell preparations.

The antibodies of the invention can be used in immunological methods that stain the cell surface in a punctate manner, suggesting that any one of the various hepsin polypeptides can be localized to specific regions of the cell surface. These microdomains, which include caveolae and shingolipid-cholesterol rafts, are believed to play critical roles in signal transduction and molecular transport. For example, GPI-anchored proteins are known to cluster in detergent-insoluble glycolipid-enriched microdomains (DIGS) of the cell surface.

### ADVANTAGES OF THE INVENTION

The physiological activator of hepsin is unknown, making the production of enzymatically active hepsin a challenge. To circumvent this problem, the present invention provides modified hepsin molecules, or fragments or derivatives thereof, each comprising a substitute activation sequence which replaces the activation sequence of a naturally-occurring, wild-type hepsin molecule. Such stably expressed modified hepsin molecules allow the activation of a modified hepsin molecule after recognition and cleavage of the modified hepsin zymogen by a desired enzyme. The modified hepsin molecules of the invention can also be used for the generation of anti-hepsin antibodies that recognize both modified and wildtype hepsin molecules.

The following examples are presented to illustrate the present invention and to assist one of ordinary skill in making and using the same. The examples are not intended in any way to otherwise limit the scope of the invention.

### EXAMPLES

### Example 1

The following sections A-G provide descriptions of methods used for detecting expression of hepsin mRNA transcripts in normal (i.e. non-prostate cancer) samples from human subjects samples from subjects with benign prostate hyperplasia and prostate cancer, as well as samples from various prostate cancer cell lines.

A) Northern Blot Analysis of Hepsin mRNA in Normal Human Tissue Samples

The following provides a description of Northern blot methods used for detecting expression of hepsin molecule mRNA transcripts in normal, human tissue samples.

Northern blot analysis of the expression of levels of hepsin mRNA in normal, human tissue was performed using multiple-tissue RNA blots from Clontech (catalogue Nos.: 7760-1, 7767-1 and 7766-1) having approximately 2 μg of poly A+ RNA from various human tissues.

A full-length hepsin probe was generated by RT-PCR amplification. Oligonucleotide primers (sense 5′-AGA GGC AGT GAC ATG GCG CAG AAG GAG GGT-3′ and antisense 5′-TGG AGG CTG CGC AGC GAG AAG-3′) were designed based on the published human hepsin cDNA sequence (Leytus et al. (1988) Biochemistry. 27 (3):1067-74). A cDNA fragment spanning the entire coding region of human hepsin was amplified from total RNA derived from human hepatoma HepG2 cells using a RT-PCR-based method (cDNA Cycle Kit, Invitrogen). PCR products were subcloned into pCR vector (Invitrogen) and sequenced. The cDNA fragment was used as a template for construction of additional plasmid vectors expressing soluble forms of human hepsin.

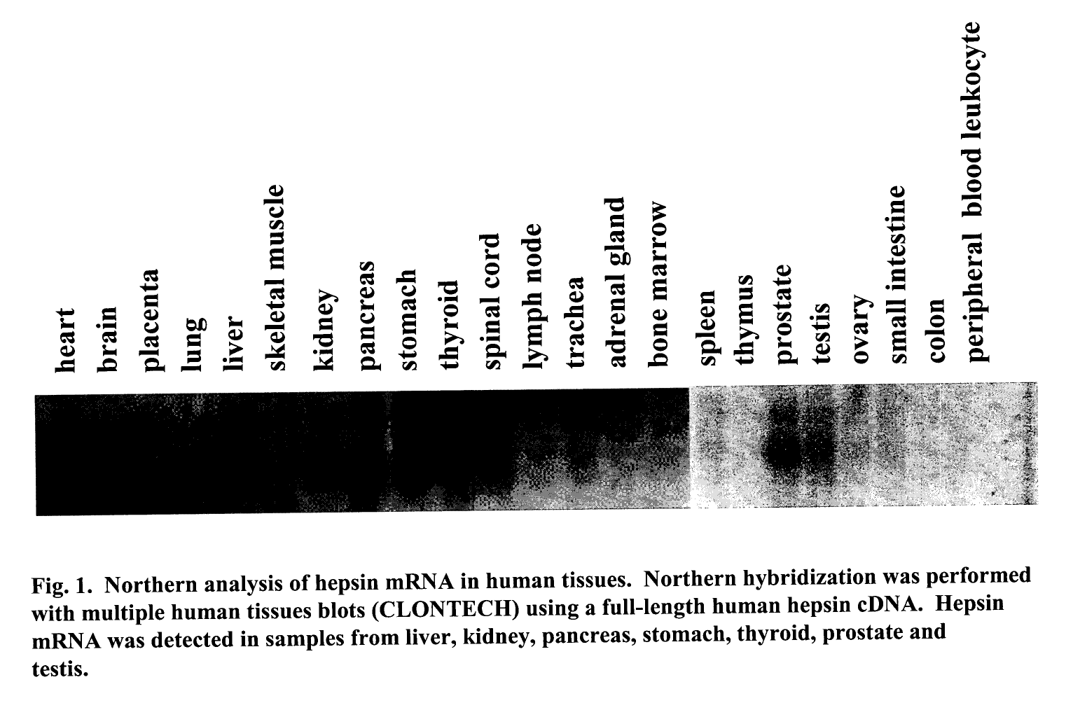

The blot was prehybridized with 10 ml Hybrisol I solution containing 50% formamide, 10% dextran sulfate, 1% SDS and blocking agents (Intergen S4040) for 2 hours at 42 degrees C. The blot was probed with a full-length human hepsin cDNA probe labeled with [α-³²P]dCTP using the Rediprime II Random Prime Labeling System (Amersham RPN1633). The blot was hybridized overnight at 42 degrees C., and washed in 2×SSC, 0.2% SDS for 30 minutes at 45 degrees C., 1×SSC, 0.2% SDS for 30 minutes at 55 degrees C., and 0.2×SSC, 0.2% SDS for 30 minutes at 60 degrees C. The blot was exposed to a phosphoimaging plate overnight, and the plate was developed in a Fuji phosphorimager (FIG. 1). As a control for sample loading, the blot was stripped and reprobed with a GAPDH cDNA probe (Clontech). The hepsin and GAPDH bands were quantitated using the Fuji MacBAS program.

The Northern blot analysis showed that hepsin mRNA transcripts were present in tissue samples derived from liver, kidney, pancreas, stomach, thyroid, prostate and testis (FIG. 1).

B) PCR Analysis of Hepsin mRNA in Normal Human Tissue Samples

The following provides a description of PCR detection methods for detecting expression of hepsin molecule mRNA transcripts in normal, human tissue samples.

RNA samples from various normal, human tissues were purchased as total RNA as indicated in the Table 1 below:

| Tissue | Source | Catalog # | Lot # |
|---|---|---|---|
| Brain | Clontech | 64020-1 | 0101041 |
| Breast | Clontech | 64037-1 | 7080713 |
| Colon | Biochain | 061003 | 8904049 |
| Heart | Clontech | 64025-1 | 607658 |
| Kidney | Clontech | 64030-1 | 7040867 |
| Liver | Clontech | 64022-1 | 7040868 |
| Lung | Clontech | 64023-1 | 7040871 |
| Lymph Node | Biochain | CDP-061035 | 8904063 |
| Pancreas | Clontech | 64031-1 | 7110156 |
| Skeletal Muscle | Clontech | 64033-1 | 7120270 |
| Spleen | Clontech | 64034-1 | |
| Stomach | Clontech | 64090-1 | 6120263 |
| Testis | Clontech | 64027-1 | |
| Uterus | Clontech | 64029-1 | |

A Taqman© (ABI Applied Biosystems) based quantitative PCR assay was used to detect the level of mRNA present in total RNA samples derived from various tissue samples. Two sets of hepsin-specific primers and probes were designed and synthesized by Atugen USA. The probes were fluorescently-labeled oligonucleotides. The 3′ ends were tamra-conjugated and the 5′ end was FAM (6-carboxy-fluorescein) labeled. Florescence was released with each PCR cycle as each hepsin amplicon was made. Quantitation was based on the increasing release of florescence with production of the amplicon at each cycle. Two sets of primer probes were used. Primer probe Set 1 was designed so the amplicon fell within an exon. Primer probe Set 2 was designed so the amplicon crossed an exon-exon boundary.

Set 1:

| | | |
|---|---|---|
| Forward primer | BLX190: 1181U21 | TCGAGTCCCCATAATCAGCAA |
| Reverse primer | BLX190: 1253L22 | CATCTTGGGCTTGATCTGG- |
| | | TTT |
| Probe | BLX190: 1204U28 | ATGTCTGCAATGGCGCTGAC- |
| | | TTCTATGG |

Set 2:

| | | |
|---|---|---|
| Forward primer | BLX190: 643U22 | AGGTCATCTCCGTGTGTGA- |
| | | TTG |
| Reverse primer | BLX190: 739L16 | CCCACGATGCGGTCCA |
| Probe | BLX190: 668U21FL | CAGAGGCCGTTTCTTGGCC- |
| | | GC |

PCR amplification was performed under the following conditions: 48° C. 30 min; 95° C. 10 min; 95° C. 15 sec and 60° C. 1 min for 40 cycles.

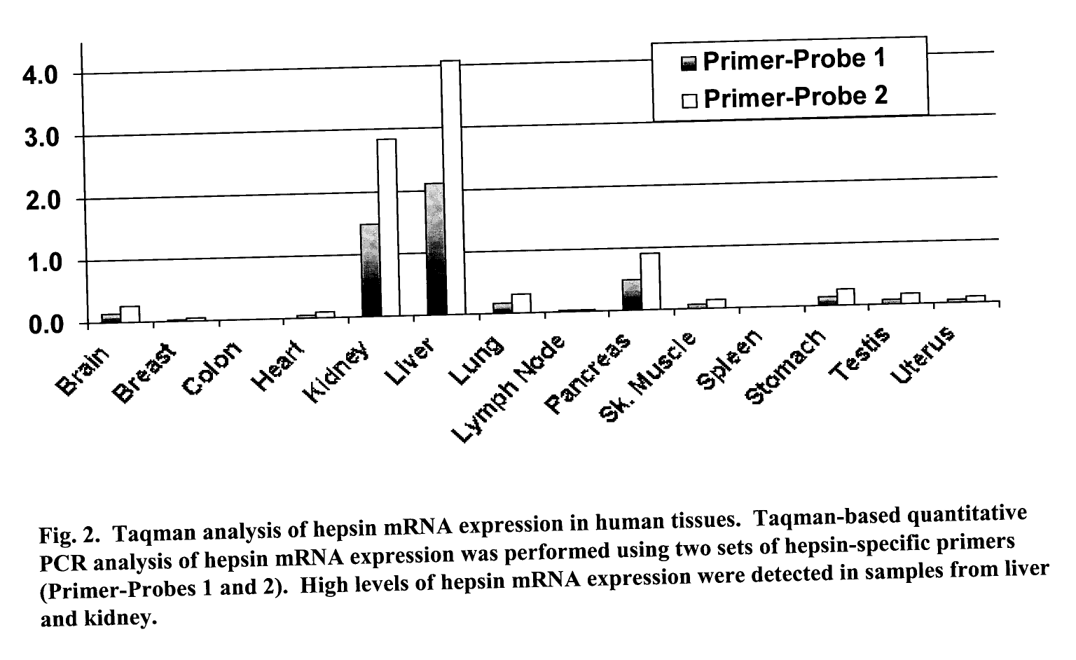

The relative PCR quantitation was performed using the ABI PRISM 7700 Detection System. The data shown in FIG. 2 is graphed as relative expression of sample hepsin transcript expression in the LNCaP sample and is based on the amount of fluorescence released per PCR cycle as the hepsin amplicon is made.

FIG. 2 showed high levels of hepsin mRNA in samples from liver and kidney as quantitated by Taqman PCR-based analysis.

C) Northern Analysis of RNA Samples from Prostate Cancer Patients

The following provides a description of Northern blot methods used for detecting expression of hepsin mRNA transcripts in normal prostate, benign prostate hyperplasia, primary prostate cancer, and advanced prostate cancer.

Total RNA samples came from prostate cancer patients. The RNA sample of normal prostate tissue were purchased from Biochain Institute Inc. The RNA samples from patients with benign prostate hyperplasia (BPH), and primary and advanced prostate cancer patients were obtained form commercial sources such as Clontech.

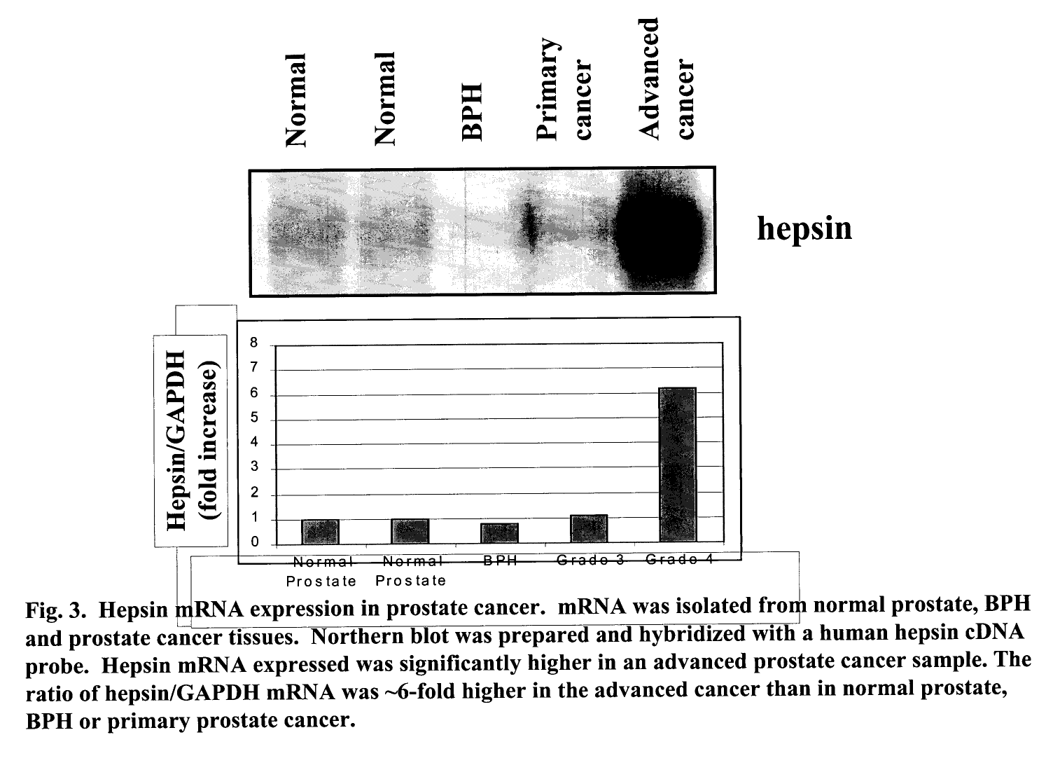

The total RNA samples were purified using the Qiagen RNAeasy method. Ten micrograms of the total RNA was denatured in formaldehyde-containing loading buffer, then separated by electrophoresis through an agarose gel. The gel was run for 4 hours at a constant 70 volts, then transferred overnight onto a nylon membrane (NEN GeneScreen Hybridization Transfer Membrane) by capillary action in 20×SSC buffer. The RNA was crosslinked onto the blot by UV exposure. The Northern blot was probed and processed as described in section A) above. The results of the Northern blot is shown in FIG. 3, upper panel.

The fold-increase of the amount of hepsin mRNA compared to GAPDH is shown in FIG. 3, lower panel.

Hepsin mRNA transcript levels were significantly higher (approximately 6-fold) in the advanced prostate cancer sample than in the normal samples, primary prostate cancer sample or benign prostate hyperplasia (BPH) sample on the Northern blot.

D) PCR Analysis of Hepsin mRNA in a Prostate Cancer Cell Line Compared to Prostate Benign Hyperplasia, and Advanced Prostate Cancer Samples

The following provides a description of quantitative PCR detection methods used for detecting hepsin mRNA transcripts in a LNCaP cell line compared to prostate benign hyperplasia, and advanced prostate cancer samples.

Total RNA samples from patients having benign prostate hyperplasia, or Gleason grade 3 or 4 prostate cancer were used. BPH=Benign prostatic hyperplaysia; GR 3=Gleason Grade 3; GR 4=Gleason Grade 4. The total RNA samples were purified using the Qiagen RNAeasy method as specified by the manufacturer.

The primer and probe sets 1 and 2, described above in section B, were used for this PCR analysis. The PCR procedure was performed according to the method described in section B above.

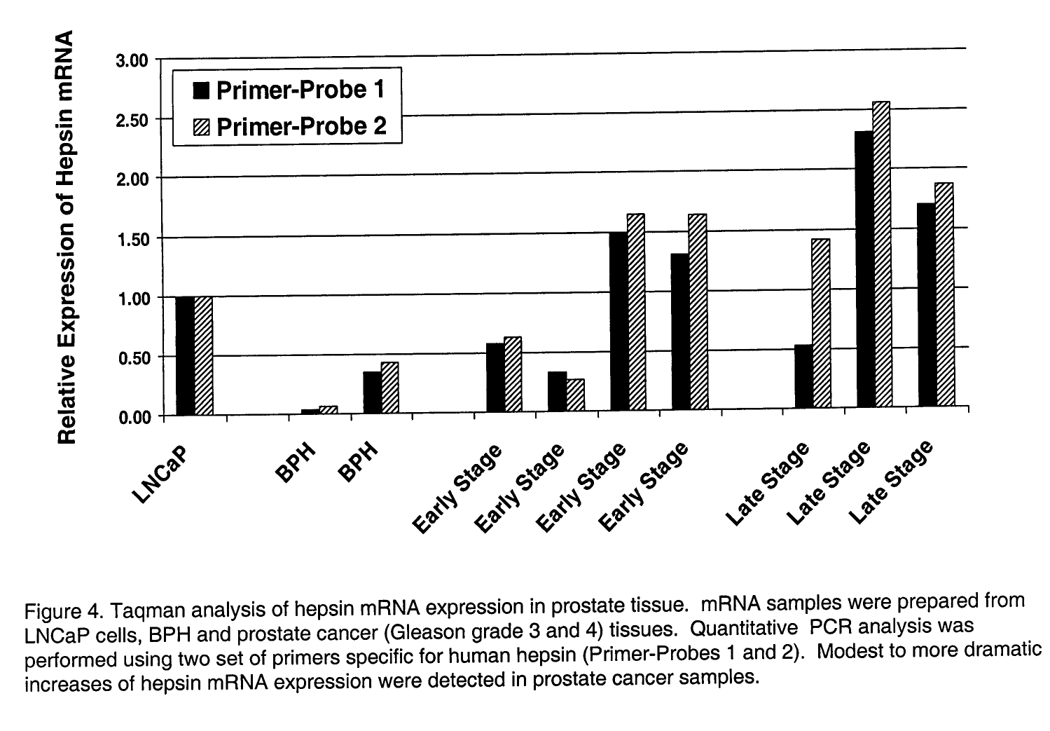

The relative PCR quantitation was performed using the ABI PRISM 7700 Detection System. The data shown in FIG. 4 is graphed as relative expression of sample hepsin transcript expression in the LNCaP sample, and is based on the amount of fluorescence released per PCR cycle as the hepsin amplicon is made.

The quantitation results showed that prostate samples from tissue staged as Gleason grade 4 tended to have higher levels of hepsin mRNA transcripts than tissue staged at a lower Gleason grade (FIG. 4).

E) Northern Analysis of RNA Samples from Prostate Cell Lines

The following provides a description of Northern blot methods used for detecting expression of hepsin mRNA transcripts in prostate cell lines.

The following cell lines were obtained from American Type Culture Collection (ATCC): PC3, DU145, HepG2, LNCaP, PZ HPV7, CA HPV10 and MDA PCa 2b. The BPH1 cells were obtained from the University of California, San Francisco.

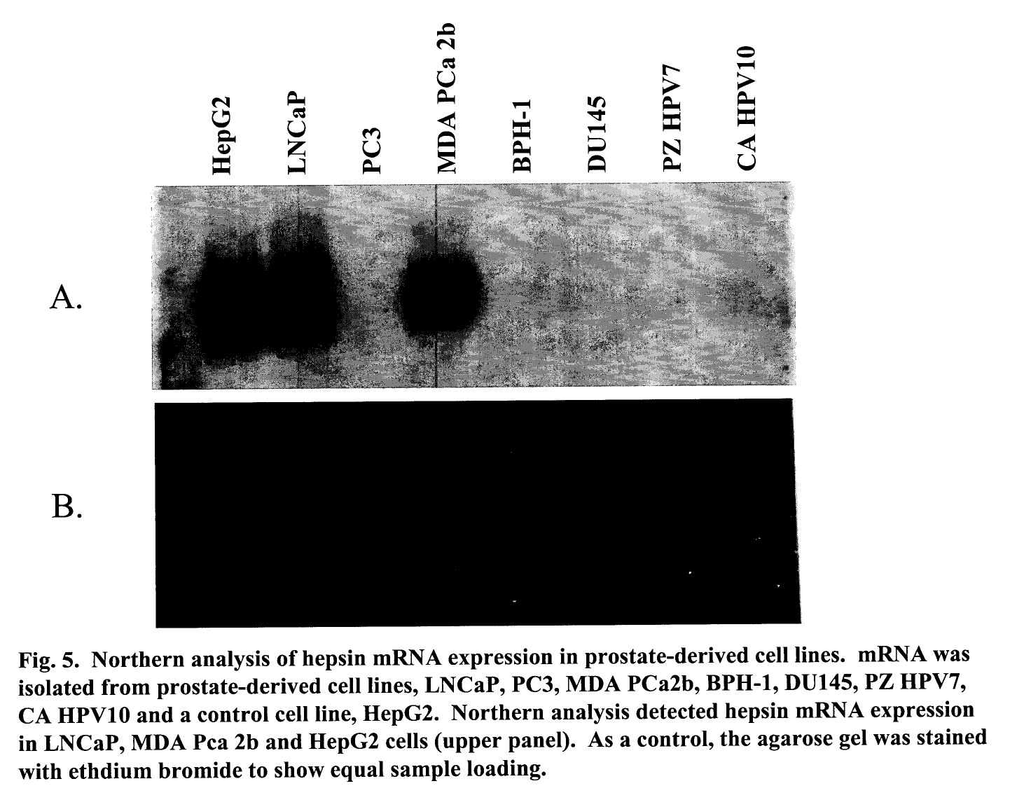

Total RNA was isolated from the cell lines using the RNeasy Maxi Kit (Qiagen). The cells were trypsinized and rinsed in PBS, then homogenized in a buffer containing guanidine isothiocyanate. An equal volume of 70% ethanol was added to the homogenate and the mixure was loaded onto a filter provided in the kit. The total RNA was immobilized on a silica gel-based membrane. This membrane was washed several times with buffer provided in the kit, then the total RNA was eluted with RNase-free water. The total RNA was precipitated with ethanol and sodium acetate and the pellet washed with 70% ethanol. The RNA pellet was resuspended in RNase-free water and quantitated. Ten micrograms of total RNA from each cell line was loaded onto an agarose gel for electrophoresis and Northern analysis as described above in section A above. The results of the Northern is shown in FIG. 5A. As a control, the agarose gel was stained with ethidium bromide to show equal sample loading (shown in FIG. 5, lower panel).

The Northern blot assay detected the presence of hepsin mRNA transcripts in LNCaP, MDA Pca 2b and the human hepatoma cell line HEPG2 cells (FIG. 5, upper panel).

F) PCR Analysis of Hepsin mRNA in Prostate Cancer Cell Lines

The following provides a description of quantitative PCR detection methods used for detecting hepsin mRNA transcripts in various prostate cancer cell lines.

The PreC cells are a normal prostate cell line purchased from Clonetics. The BPH1 cells were obtained from Dr. Cuhna at UCSF. The cell lines PC3, DU145, and MDA PCa 2b cells are from ATCC.

The PC-3 cells were initiated from a bone metastasis of a grade IV prostatic adenocarcinoma from a 62-year-old male Caucasian, and are androgen independent. MDA PCa 2b cells were established from a bone metastasis of 63-year-old Black male with androgen-independent adenocarcinoma of the prostate. LNCaP cells were established from a Lymph node metastasis of 50-year-old Caucasian male with androgen-dependent adenocarcinoma of the prostate. Total RNA was purified from the tissue using the Qiagen RNAeasy kit.

The primer and probe sets 1 and 2, described above in section B, were used for this PCR analysis. The PCR procedure was performed according to the method described in section B above.

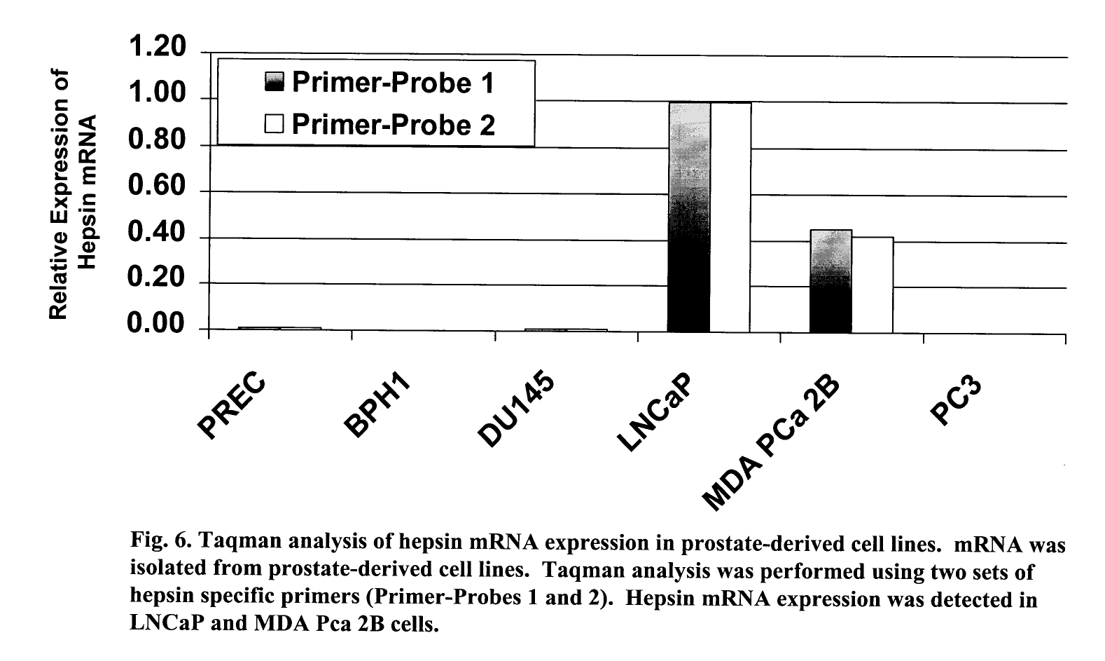

The relative PCR quantitation was performed using the ABI PRISM 7700 Detection System. The data shown in FIG. 6 is graphed as relative expression of sample hepsin transcript expression in the LNCaP sample, and is based on the amount of fluorescence released per PCR cycle as the hepsin amplicon is made.

PCR analysis with two sets of primer probes detected hepsin mRNA transcripts in LNCaP and MDA Pca 2B cells (FIG. 6).

G) Northern Analysis of RNA Samples from Cells Treated with DHT

The following provides a description of the Northern blot methods used for detecting expression of hepsin mRNA transcripts in cells treated or untreated with dihydrotestosterone (DHT).

The prostate cancer cell lines, LNCaP, were obtained from ATCC. The LNCaP cells were treated with dihydrotestosterone (Sigma A 8380) in order to study the response of hepsin mRNA expression to androgens. The confluent cells were cultured in growth medium containing charcoal-stripped FBS for 24 hours. DHT was added to each flask at 10 nM. For control cells, ethanol was added as a vehicle control. The cells were incubated for 24 or 72 hours.

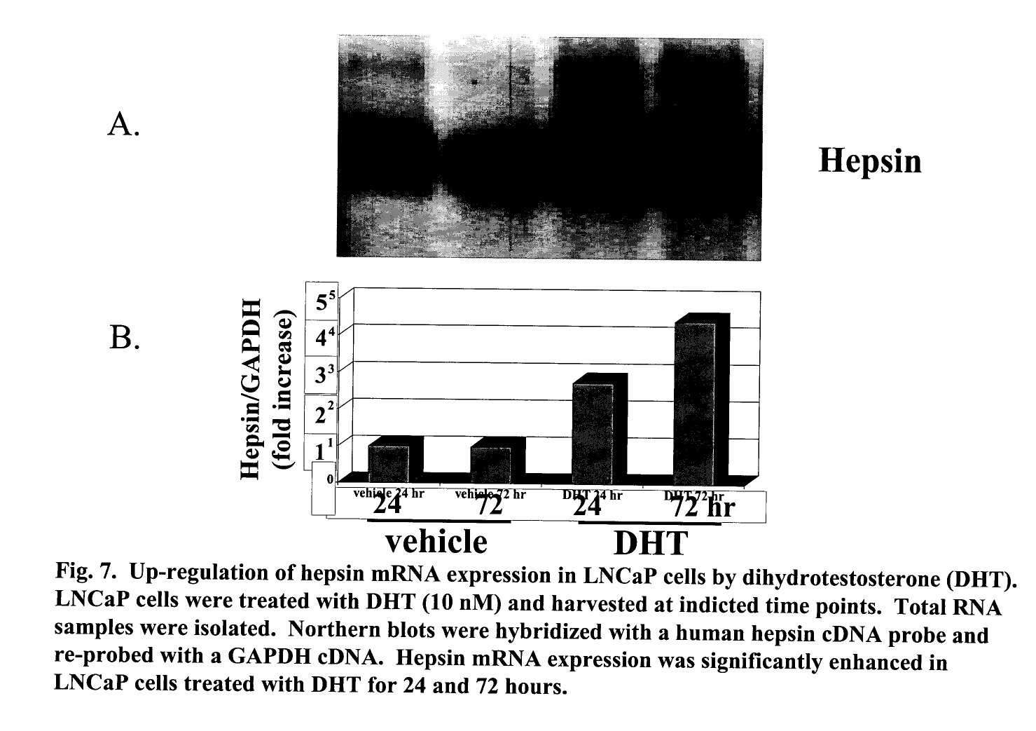

Total RNA was isolated from the cell lines using the RNeasy Maxi Kit (Qiagen), as described in section E above. The Northern gel and blot methods were performed as described in section A above. The results are shown in FIG. 7A.

The fold-increase of the amount of hepsin mRNA compared to GAPDH was performed according to section C above. The results, shown in FIG. 7B, indicated that hepsin mRNA transcipt levels were increased in LNCaP cells treated with DHT.

### Example 2

The following provides descriptions of the methods used to amplify human hepsin cDNA and to generate recombinant DNA molecules encoding modified hepsin molecules.

Amplification of Human Hepsin cDNA:

Oligonucleotide primers (sense 5′-AGA GGC AGT GAC ATG GCG CAG AAG GAG GGT-3′ and antisense 5′-TGG AGG CTG CGC AGC GAG AAG-3′) were designed based on the published human hepsin cDNA sequence (Leytus et al. (1988) Biochemistry. 27 (3):1067-74). A cDNA fragment spanning the entire coding region of human hepsin was amplified from total RNA derived from human hepatoma HepG2 cells using a RT-PCR-based method (cDNA Cycle Kit, Invitrogen). PCR products were subcloned into pCR vector (Invitrogen) and sequenced. The cDNA fragment was used as a template for construction of additional plasmid vectors expressing soluble forms of human hepsin.

Plasmid pAcGP67/hepED

Cloning of Hepsin Full Length ecto-Domain, i.e., the extracellular domain which includes the protease domain and scavenger receptor domain, was performed. The hepsin extracellular domain was cloned with a V5 and His tag at the carboxy end into the baculovirus transfer vector pAcGP67a (pAcGP67, from Pharmingen, is available in three reading frames; frame pAcGP67a was used).

PCR amplification of the V5 and 6His tag from pcDNA 3.11V5His (Invitrogen, San Diego) was performed using the upstream primer (V5HisFor—5′ CAGCTCGAATTCGGTAAGCCTATCCCT 3′) and the downstream primer (V5H is Rev-5′GATGCGGCCGCTTTAAACTCAATGGTG 3′).

PCR amplification of hepsin was performed using the upstream primer (NXhpsnfor—5′ CATATGCCCGGGAGGAGTGACCAGGAG 3′), the downstream primer (hpsnrev—5′ CTTACCGAATTCGAGCTGGGTCACCAT 3′).

PCR amplification was performed under conditions: 94° C. 1 cycle; 94° C. 30 sec, 68° C. 1 min 40 cycles; 70° C. 7 min 1 cycle; 4° C. hold. Advantage polymerase (proofreading) was used (Clontech) as per manufacturer's suggested protocol.

The amplicons, vector and insert, were run on a 1% TAE agarose gel and gel-isolated. Gel isolated fragments, vector and insert, was digested with NotI/XmaI. The vector and insert fragments were further purified, to remove digested ends, then ligated into the NotI/XmaI site in pOUT10 (i.e. pAcGP67a from Pharmingen). The recombinant plasmid was used to transform DH5α cells.

Plasmid pAcGP67/hepED/EK

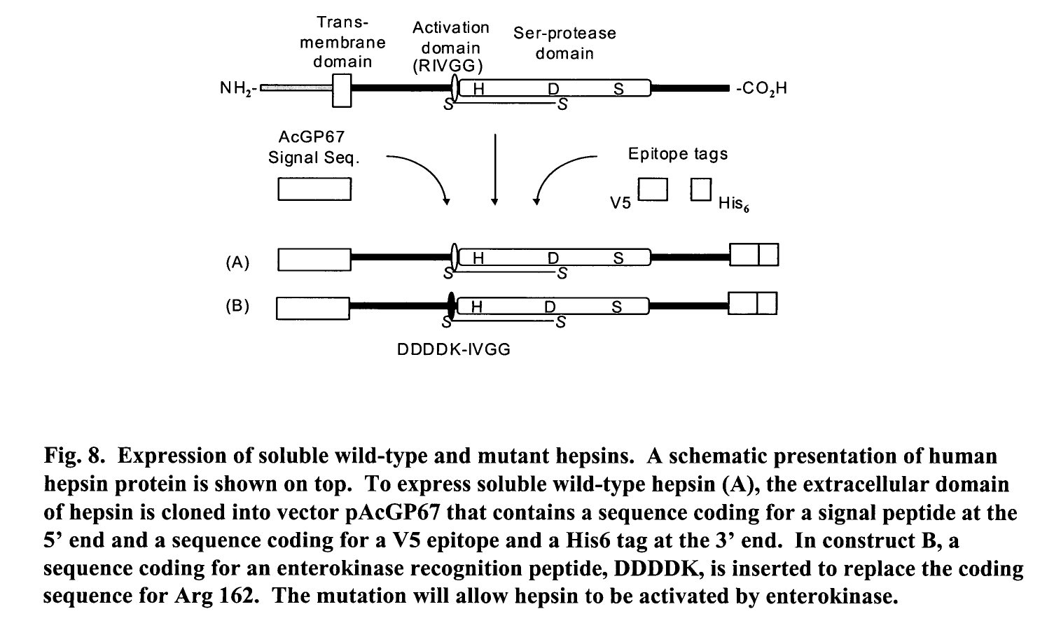

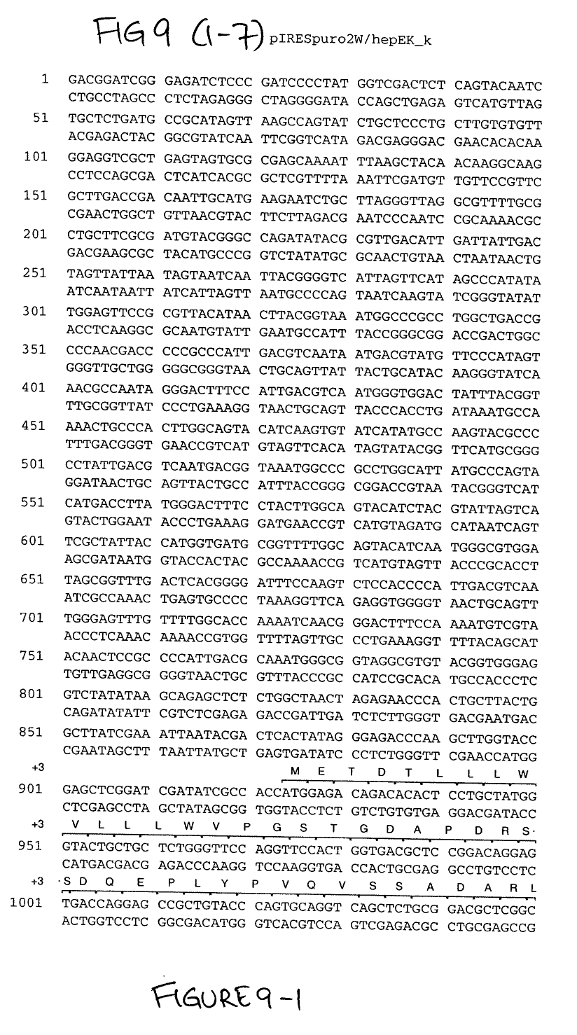

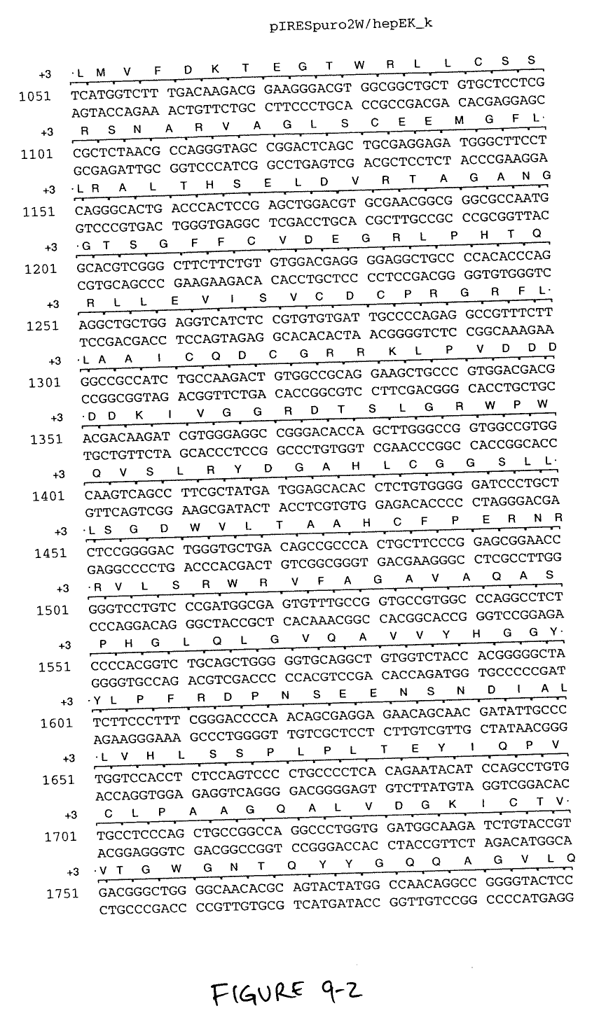

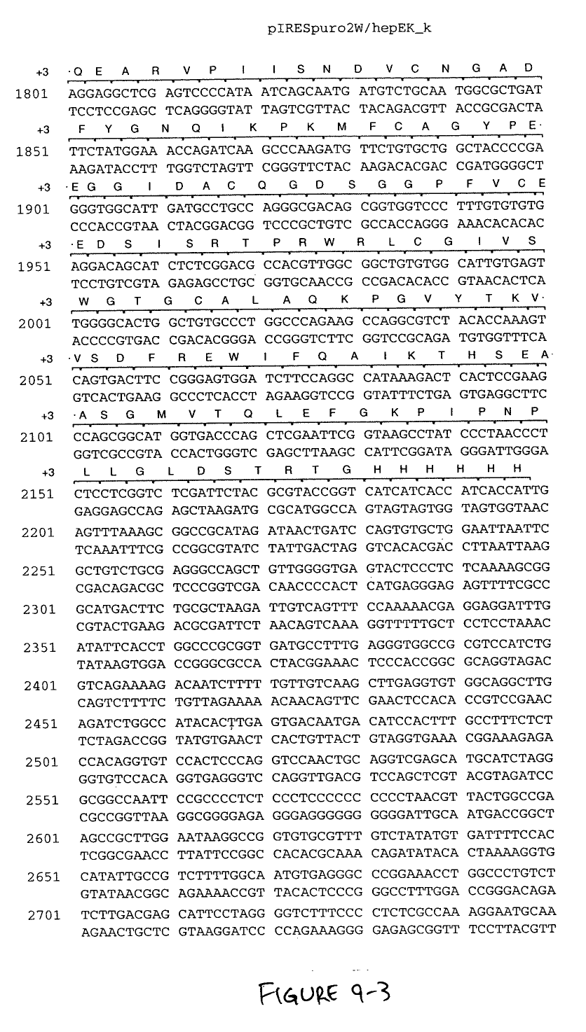

A recombinant DNA molecule, pAcGP67/hepED/EK, encoding the extracellular domain of hepsin and an enterokinase cleavage site was generated by the cloning method described infra. A schematic diagram of the recombinant plasmid is shown in FIG. 8.

The Hepsin EDEK sequence (Hepsin ectodomain with enterokinase recognition sequence) was cloned into the pIVEX (Roche Molecular Biochemicals) by PCR amplification of the hepsin fragment, subsequent restriction enzyme digestion of the fragment and ligation into the pIVEX vector. The hepsin EDEK insert in pIVEX hepsinEK was subcloned into pAcgp67 (Pharmingen) at the XmaI/NotI sites.

The pAcGP67-hepsin EK was transfected into insect cells using Baculogold transfection kit (Pharmingen) and virus isolated.

The expression of the hepsin molecule encoded by the plasmid construct was examined by western blot using anti-His antibody.

Plasmid pIRESpuro2W/hepEK

The plasmid construct pIRESpuro2W/hepEK having a hepsin insert was generated in order to express hepsin ED/EK in CHO cells (FIG. 9). (ED=ectodomain=extracellular domain; EK=enterokinase cleavage site)

The cDNA encoding soluble hepsin ED/EK was amplified as a PCR product with the primer pair of hepBspE1_F (CTGATCCGGAcAGGAGTGACCAGGAGCCGC) and hep_R2 (GCCGGGTC CCAGGAAAGGA). pAcGP67/hepED/EK, described supra, served as template. The PCR product was digested with BspEI+NotI for cloning into the expression vector pIRESpuro2W described infra. This PCR fragment includes hepsin ED/EK and two tags: V5 and 6-His.

An Igκ signal sequence was PCR amplified from pSecTag2A (Invitrogen) using the following primers: Igκ_F (gatcgatatcgccaccatggagacagacacactcctgctat gggtactgctgctctgggttccagg) and Igκ_R (atcgTCCGGAGCGTCACCAGTGGAACCT GGAACCCAGAGCAGCAGt). EcoRV and BspEI were used to create compatible ends for ligation.

pIRESpuro2W (a derivative of pIRESpuro2 that was originally purchased from ClonTech, modified in house) was linearized with EcoRV/NotI and used as vector backbone.

pIRESpuro2W/hepEK was constructed by three-way ligation (Fast-Link DNA ligation kit, Epicentre) of the restricted PCR fragments described above (Igκ signal sequence, hepsin ED/DK) into pIRESpuro2W.

Plasmid pCEP4W/hepEK

Plasmid pCEP4 (Invitrogen) was modified by inserting woodchuck hepatitis virus post-transcriptional regulatory element (WPRE) at the XhoI site. This modified plasmid was designated pCEP4W.

The KpnI-hepEK—NotI fragment from pIRESpuro2W/hepEK was cloned into the KpnI/NotI site of pCEP4W to create pCEP4W/hepEK (FIG. 10) for transient expression of hepsin ED/EK in 293EBNA cells (Edge Biosystems).

Plasmid pCEP4W/hepEK36 (i.e., pCEP4W/hep36)

A cDNA fragment encoding the serine protease domain of soluble hepsin ED/EK(hep36) was PCR amplified from pAcGP67/hepsin ED/EK using primers hep36_F (GAGATCCGGACCAAG ACTGTGGCCGTAGGAAGCTG) and hep36_R (GCCGGGTCCCAGGAA AGGA). The hepsin ED/EK fragment (from BspEI to NotI) in pCEP4W/hepEK was replaced by the PCR product of hep36 to create the construct pCEP4W/hepEK36 (FIG. 11).

pBACSurf-Hepsin-gp64

Hepsin ED was cloned into the KpnI site of pBacSurf1 (a baculovirus transfer plasmid from Novagen). This construct results in the fusion of the hepsin ectodomain to gp64.

Primers were synthesized that place flanking KpnI sites on the Hepsin ectodomain: srfhepfor2 (5′ TGCAGGTACCTAGGAGTGACCAGGAGCCGCTG 3′); srfheprev2 (5′ CCGGGGTACCAGCTGGGTCACCATGCCGCTGGC 3′).

The PCR amplification reaction using the primer was performed with the following conditions: 94° C. 4 minutes lx; 94° C. 30 seconds, 68° C. 2 minutes, 40×; 68° C. 10 minutes 1×. The reaction buffer contained Clontech taq polymerase cDNA buffer.

When the transfer plasmid recombines with wild-type virus, the gp64-hepsin protein is coexpressed with gp64. The wild-type virus produces the envelope glycoprotein gp64. Recombination of the transfer plasmid with the wild-type viral DNA results in the production of the gp64-fusion protein because it is driven by a separate and independent promoter. Therefore, both gp64 and gp64-hepsin proteins are expressed. The molecules expressed from this plasmid were later used to immunize mice for antibody production.

### Example 3

The following provides a description of the methods used to produce and isolate the modified hepsin molecule in eucaryotic cells.

Expression of Hepsin ED/EK and Hepsin Proteinase Domain in 293EBNA Cells

Transient transfections of 293EBNA cells (Edge Biosystems) was performed with pCEK4W/hepEK and pCEK4W/hepEK36.

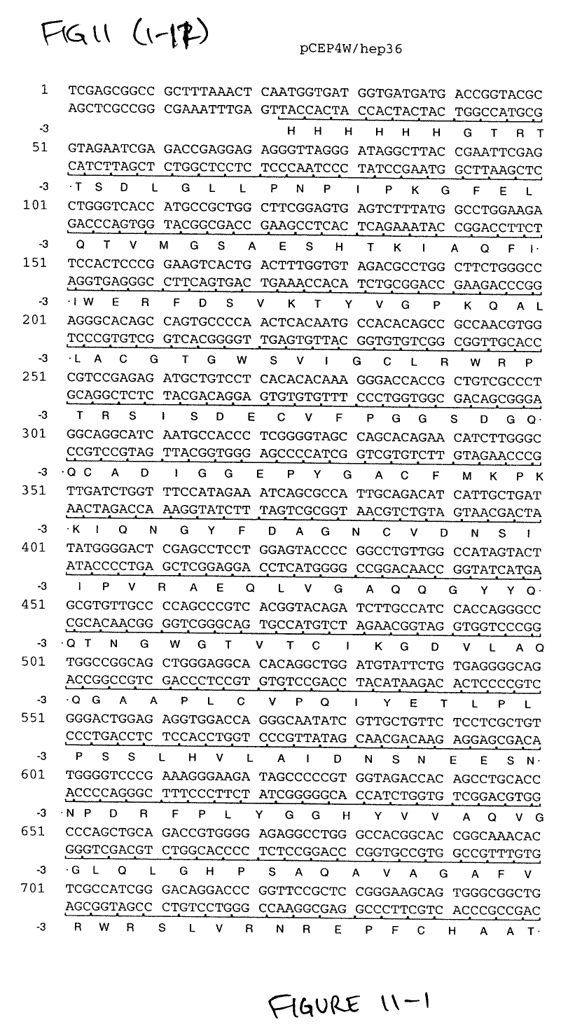

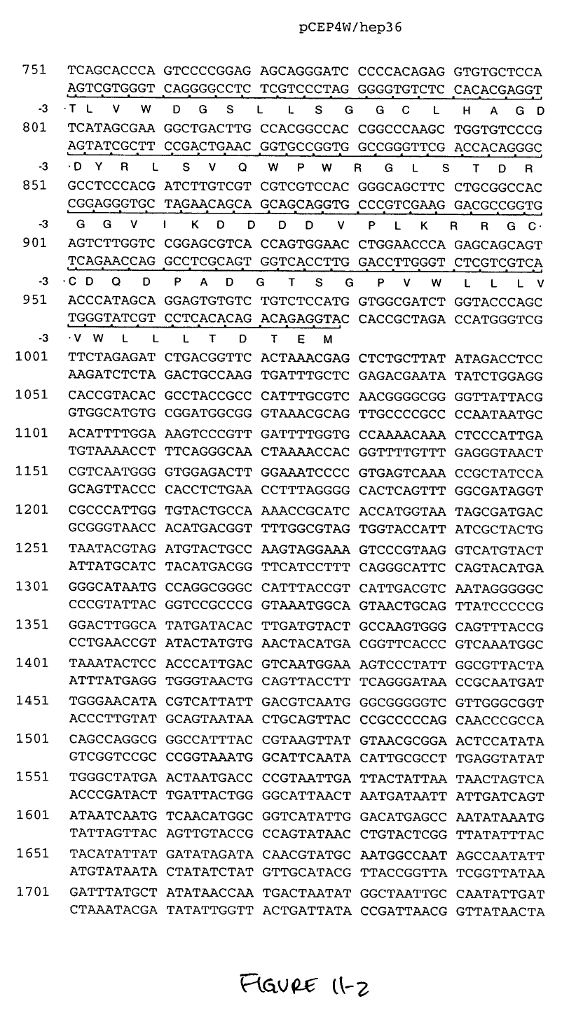

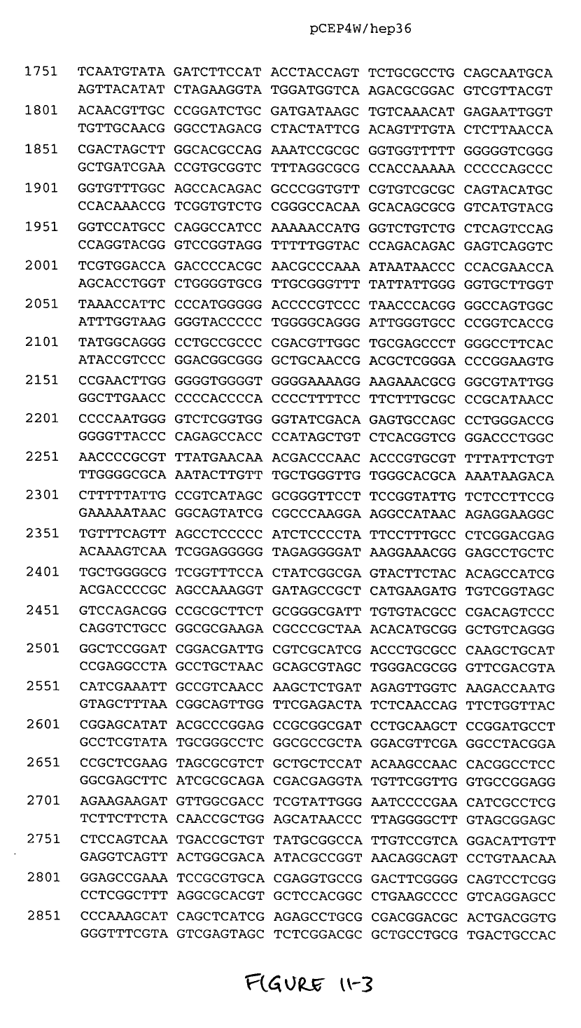

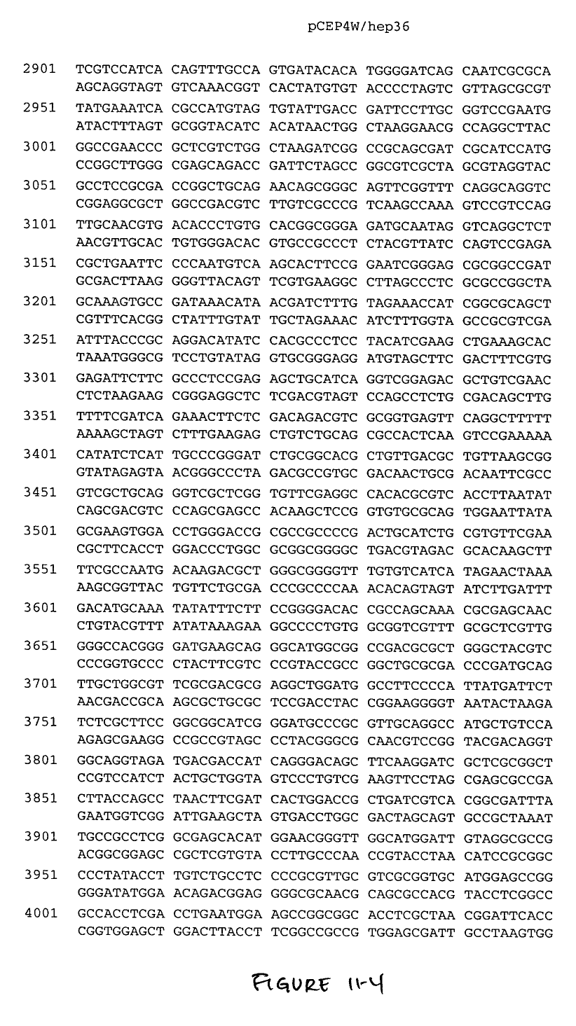

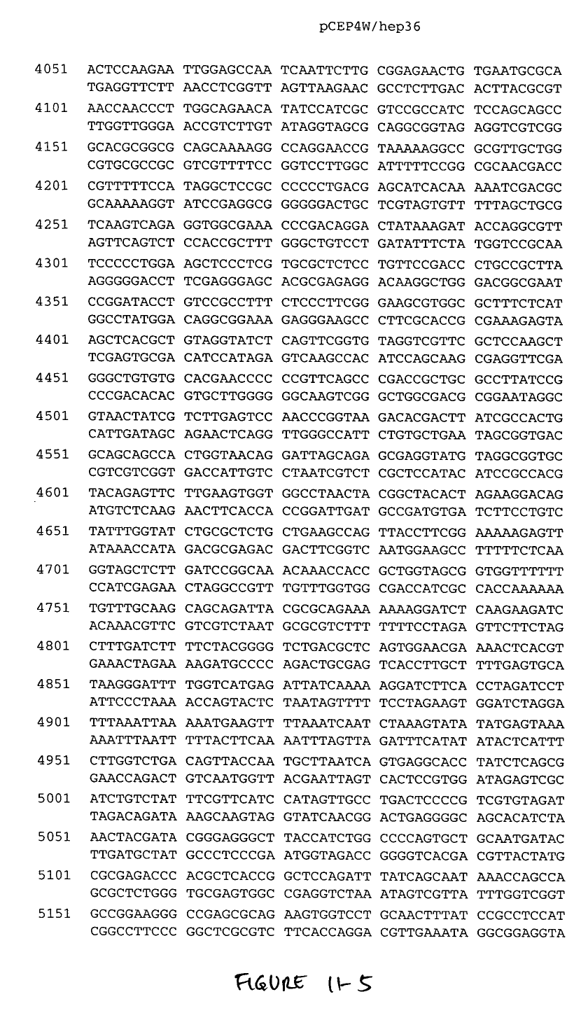

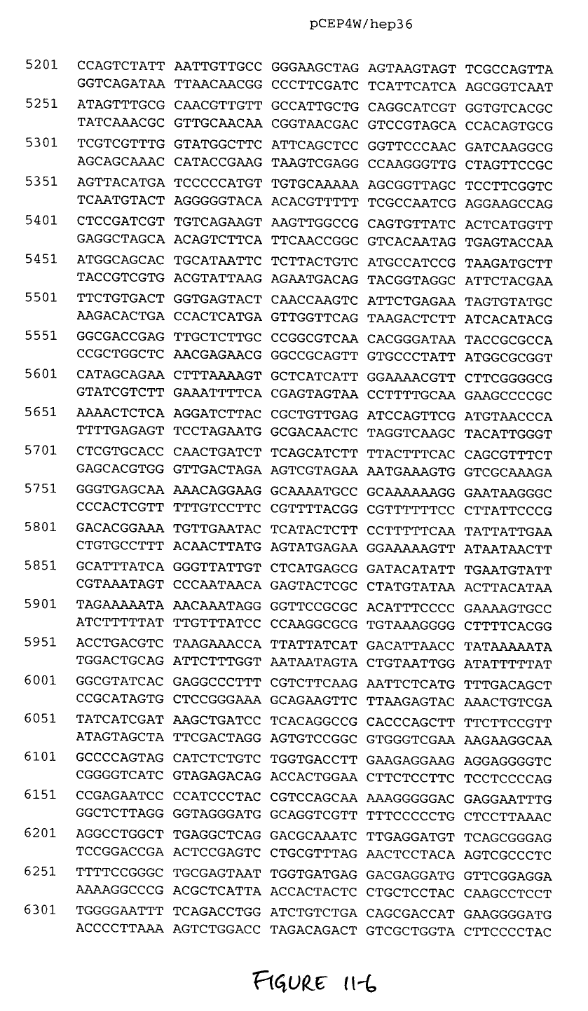

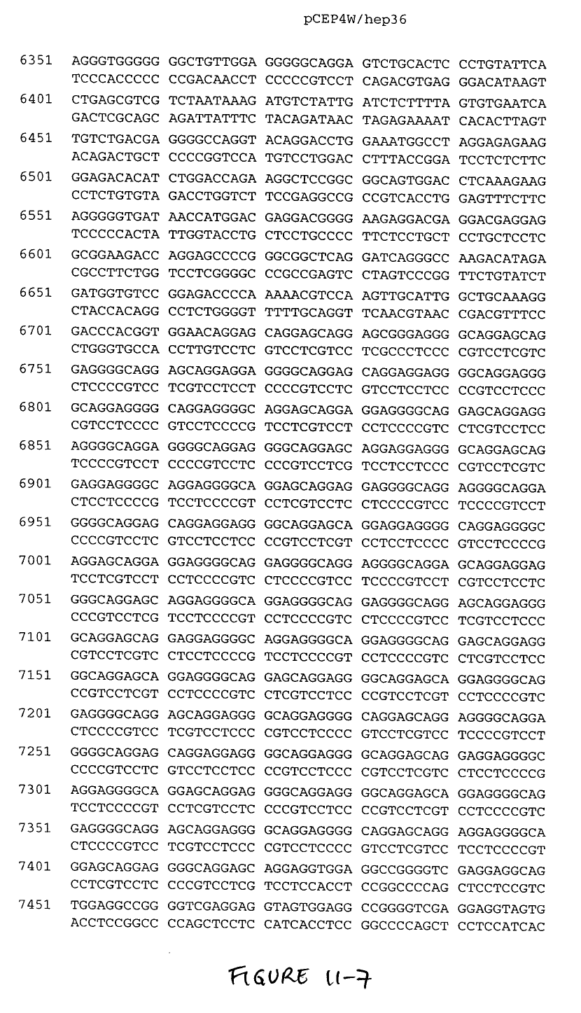

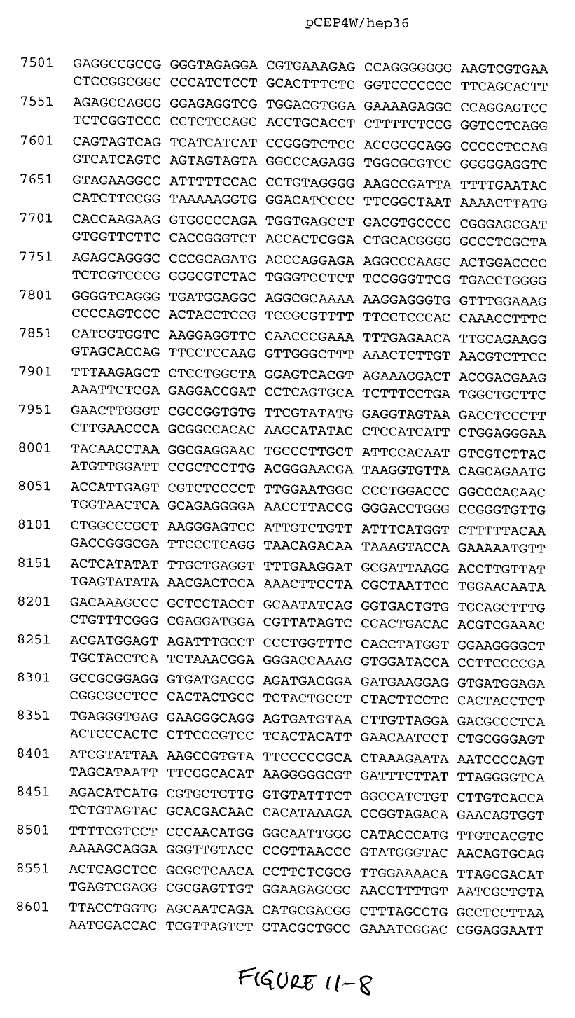

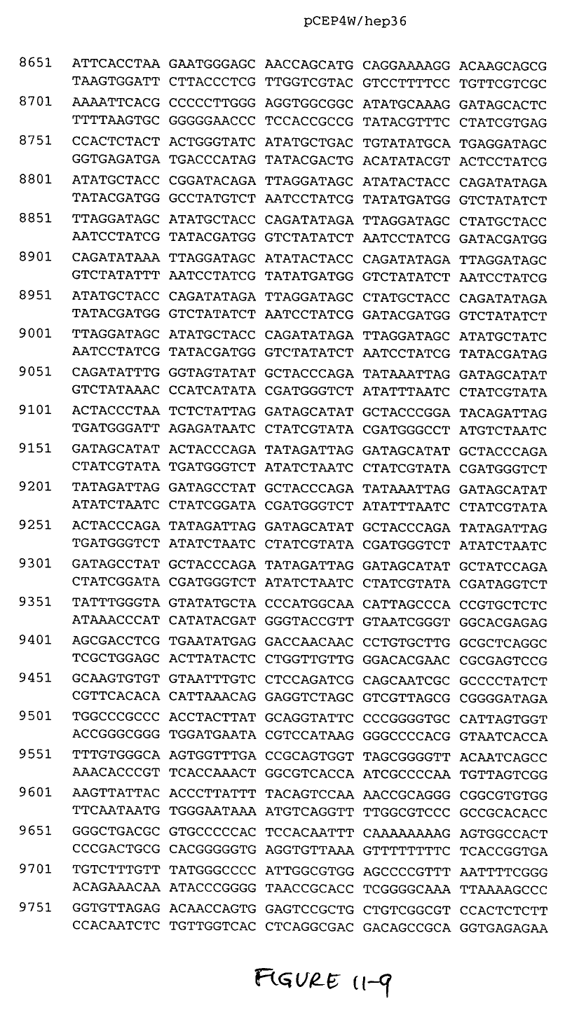

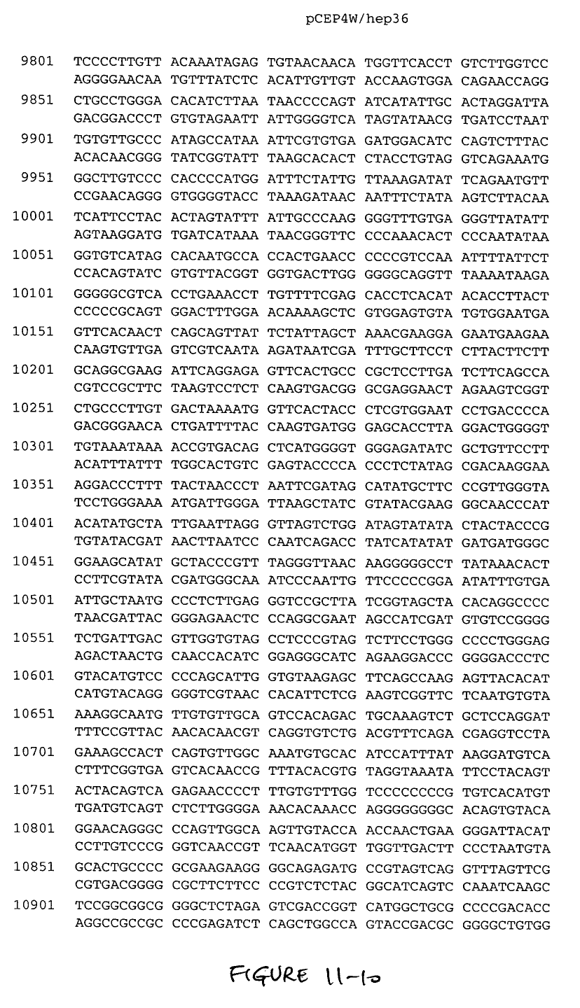

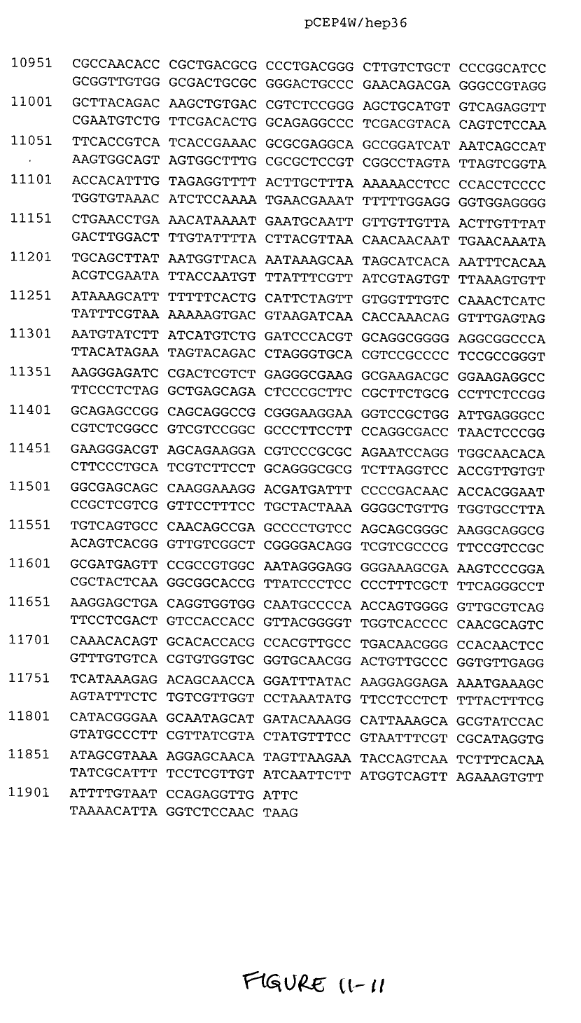

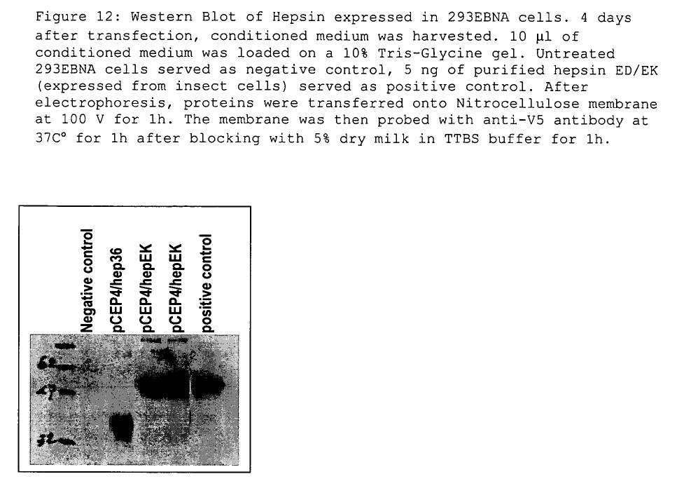

Suspension cultures of 293EBNA cells were maintained in 293SFMII (LTI Cat. No. 11686086) in spinner flasks. On the day of transfection, cells were washed twice with Ca⁺⁺ free DMEM, and diluted to a density of 0.5×10⁶ cells/ml×1L into 3L spinners with transfection medium (Ca⁺⁺ free DMEM supplement with 2% FBS, 2 mM L-Glutamine and 1 mM Sodium pyruvate). Cells were returned to the incubator and ready for transfection. All the cells and cell culture material were from Invitrogen. Transient transfection was performed with X-tremeGENE Ro1539 transfection reagent (Roche) following the manufacturer's protocol. Basically, mix 0.4 mg of DNA with 100 ml of DMEM Ca⁺⁺ free medium; add 1 ml of Ro1539 2 minutes later. DNA/Ro1539 mixture was then incubated at room temperature for 40 minutes to allow complex formation. Mix again, and add the mixture to the preprepared cells. Incubate the cells at 37C° for 4 days before harvesting the expressed hepsin (FIG. 12).

The suggested DNA/lipid ratio in 12-well plates is 0.8/8/well, while in spinners, 0.2-0.4 ug per ml of cells. Optimization can be done in 12-well plate by varying the ratio.

Once the cells are transfected, suspension cultures of the HEK-293EBNA cells can be maintained in 293SFMII supplemented with 4 mM L-glutamine.

Expression of Hepsin Using a Baculovirus-Mediated System in Insect Cells

Cell Transfection

Plasmids pAcGP67-HepsinED and pAcGP67-HepsinEDEK were used to transfect insect cells Sf21.

Insect cells Sf21 were plated in a 6 well plate at a concentration of 1E6 (i.e. 10⁶) in Grace's medium (Invitrogen, Catalog # 11605) plus 5% FBS. The cells were allowed to attach to the plates by incubating the cells for ½ hour at room temperature.

Using the BaculoGold kit (Pharmingen) for transfection of the baculovirus transfer vector comprising a hepsin molecule, media from two of the wells were removed and replaced with 0.5 ml of solution A.

In a sterile microfuge tube, 2 μg plasmid DNA (pAcGP67-HepsinED or pAcGP67-HepsinEDEK) was mixed with 5 μl of linear baculovirus DNA provided in the kit. The mixture was allowed to stand for 5 minutes before adding 0.5 ml of solution B. The viral mixture was diluted in Grace's medium and added dropwise to the cells in solution A. The plates were placed on a rocking platform for 1-4 hours at room temperature. After which, the media was removed from the cells and 1% agarose in 2 mls of Grace's medium with 5% FBS was added to each of the wells. The cells were incubated at 27° C. for 3-6 days or until plaques appear.

After 5 days a sample of the supernatant was collected. The concentration of the recombinant virus was titered and viral plaques were picked to infect Sf21 insect cells in a 6 well plate.

Assay for Expression

After 4 days the infected cells and supernatant was collected. The pellet was resuspended in the original volume using PBS. An equal volume of 2× sample buffer with 1 mM DTT was mixed with 5 μl of the cell suspension or supernatant. The samples were boiled and loaded on a 4-20% gel SDS Page and a Western Blot was generated.

Western Blot

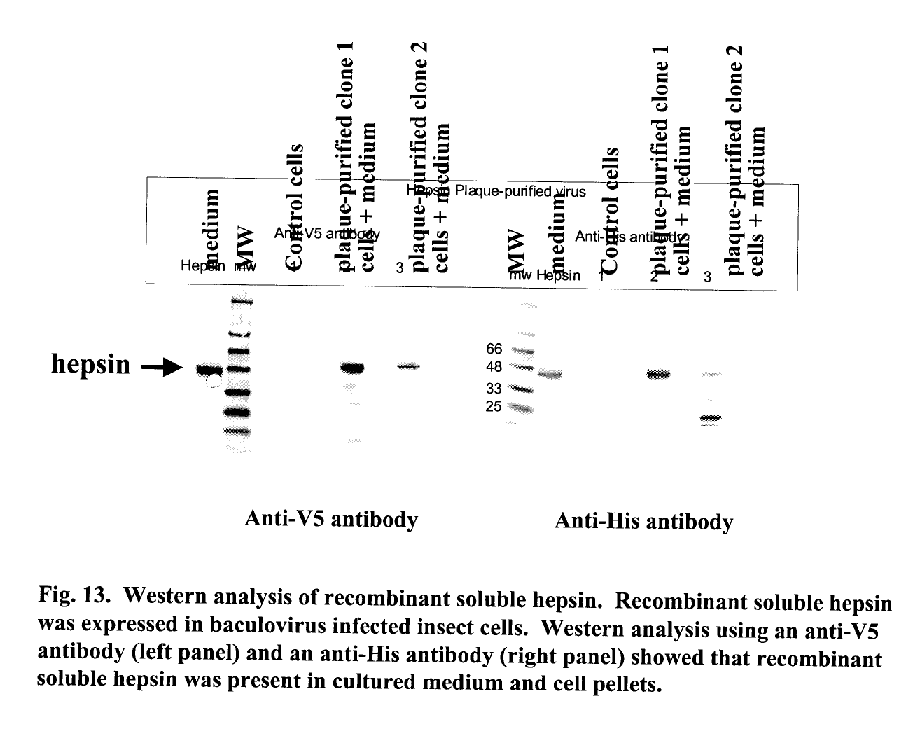

A Western blot, as shown in FIG. 13, was generated in the following manner in order to analyze the hepsin expressed by baculoviral infection of insect cells.

Harvest 50 μl of condition medium from the cells, spin 1 minute at the highest speed in bench top centrifuge, transfer the supernatant to a fresh tube, add 50 μl of SDS sample buffer, vortex, heat to 95C° for 5 minutes. Load up to 20 μl of samples per well on to a Tri-glycine gel in SDS running buffer (0.025M Tris Base, 0.192M glycine, 0.1% SDS)). Run gel at 180V until the bromophenol dye runs to the end of the gel. Place the gel next to a nitrocellulose membrane and transfer the protein from the gel to the nitrocellulose by running an electrical current of 300 mAMP through the Western transfer apparatus (XCELL II, Invitrogen) for 45 minutes. Block 1 hour with 5% dry milk in TTBS.

The Western blot was probed by exposing the blot to an anti-V5 conjugated to horse-radish-peroxidase (anti-V5-HRP) (1:5000 dilution in TTBS+1% BSA) for 1 hour. Wash the blot three times with TTBS at 10 minutes/each wash, then TBS once for 10 minutes. Develop the Western blots by incubating the blots in 10 ml of each ECL reagent (Amersham Pharmacia Biotech) for 1 minute, expose to film for a minimum of 30 minutes (FIG. 13, left panel).

The results of the of the expression and isolation of recombinant hepsin are shown in the Western blot of FIG. 12, where recombinant hepsin protein was shown to be expressed in baculovirus infected insect cells.

### Example 4

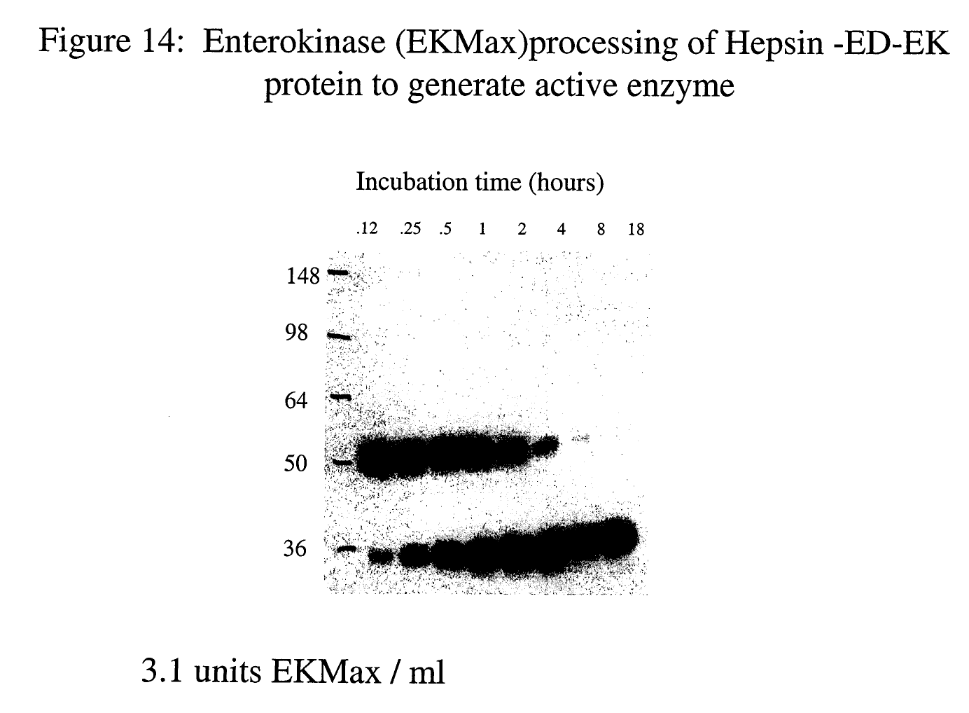

The following provides a description of the methods used to activate the modified hepsin molecule, as shown in FIG. 14, with enterokinase treatment to produce an activated modified hepsin protease and methods used to detect activated hepsin.

Activating Hepsin with Enterokinase

The modified hepsin molecule purified on the Phenyl-HIC column, as described above in Example 4, was in a final eluent of approximately 330 mM ammonium sulfate, 150 mM sodium chloride, 50 mM sodium acetate pH 5.5 (HIC eluent). This was diluted 1:2 in 150 mM sodium acetate pH 5.5 and concentrated to approximately 0.3 g/L.

The concentrated modified hepsin molecule was treated with enterokinase (Roche Diagnostics GmbH 1 351 311) at room temperature overnight, about 22 hours, at a mass ratio of 85:1 modified hepsin molecule to enterokinase. The enterokinase can be removed or can remain after the activation reaction.

The modified hepsin molecule can be activated by enterokinase treatment under a variety of conditions (pH 4.5-7.5). The lower pH results in less auto-degradation.

The activated, modified hepsin molecule was diluted to 100 nM in 100 mM Hepes, 100 mM NaCl pH 7.4, and 1.25 ml aliquots were placed in cryovials and the vials were flash frozen in liquid nitrogen for storage at −80 degrees C.

Detecting Activated Hepsin

The physiological substrate(s) of hepsin is not known (Wu, Frontiers in Bioscience 2001;6:d192-200), however hepsin was reported to activate blood coagulation factor VII (FVII) in vitro (Kazama et al. J. Biol. Chem. 1995;270:66-72).

A cell-based assay was established to detect the activity of hepsin on the cell surface. In this assay, human recombinant hepsin was stably expressed in baby hamster kidney (BHK) cells. The hepsin expressing cells (˜8×10⁵) or control BHK cells were incubated in a phosphate-beffered saline (pH 7.4) containing purified human plasma FVII (0.2 μg/ml) at 37° C. for 30 min. Conversion of FVII to FVIIa was analyzed either by Western blotting using a sheep anti-human FVII antibody to detect the cleavage of the molecule or by a chrombogenic substrate (S-2266, H-D-Val-Leu-Arg-pNA-2HCl)-based assay to detect the activity of FVIIa. This assay measures the activity of hepsin on the cell surface and can be used to screen inhibitors that interact with cell surface hepsin.

### Example 5

The following provides a description of the methods used to produce and characterize antibodies that bind the modified hepsin molecule.

Generating Antibodies Against Hepsin-ED-EK Protein

An immunogen, Hepsin-ED-EK, comprising native (i.e:, nondenatured) Hepsin ectodomain having an enterokinase cleavage site, V5 tag and 6×His tag was expressed in insect cells from plasmid pAcGP67/hepED/EK as described supra. The protein was purified using an affinity column with anti-V5 bound to Sepharose.™.

Two Hepsin Knock-out mice (U.S. Pat. No. 5,981,830), one female and one male, were immunized with native Hepsin-ectodomain using the Rapid Immunization Multiple Sites (RIMMS) method (Kilpatrick, K. E., et.al., 1997, Hybridoma, Volume 16, Number 4). The mice were initially immunized with 10 μg Hepsin/mouse in RIBI adjuvant (ImmunoChem Research, Inc.), then boosted four times with 51 μg Hepsin/mouse. Then the female mouse immunization was boosted an additional two times with native Hepsin-ED-EK Protein. Female mouse #1 protein immunized was chosen for fusion. The male mouse's immunization was boosted two times with SDS-Denatured Hepsin-ED-EK protein (the protein was denatured in 1% SDS with 10 min and boiling at 90° C.). All boosts were done with the RIMMS method.

The immunogen i.e., antigen (Ag), was injected sub-cutaneously at twelve sites proximal to the draining lymph nodes according to the following schedule:

Hepsin-ED-EK Protein Immunization Schedule

| | |
|---|---|
| Day 1: | Pre-Bleed, number mice and perform |
| | the 1^{st} immunization 10 μg/mouse |
| | RIMMS (65 μl Hepsin Protein, |
| | +500 μl RIBI 2x, +500 μl NaCl.) |
| Day 4: | 1^{st} boost, 5 μg/mouse, RIMMS (35 μl |
| | Hepsin Protein, +500 μl RIBI 2x, +500 μl NaCl.) |
| Day 6: | 2^{nd} boost, 5 μg/mouse, RIMMS (35 μl |
| | Hepsin Protein, +500 μl RIBI 2x, +500 μl NaCl.) |
| Day 8: | 3^{rd} boost, 5 μg/mouse, RIMMS (35 μl |
| | Hepsin Protein, +500 μl RIBI 2x, +500 μl NaCl.) |
| Day 11: | 4^{th} boost, 5 μg/mouse, RIMMS, (35 μl |
| | Hepsin Protein, +500 μl RIBI 2x, +500 μl NaCl.) |
| | Tail vein bleed. |

Generating Antibodies Against Hepsin-ED-gp64 Fusion Protein

A Baculovirus surface expressed Hepsin i.e., hepsin-ED-gp64 fusion protein, was expressed using the pBACSurf-Hepsin-gp64 recombinant plasmid described in Example 2, supra. The fusion protein was isolated using an affinity column with anti-V5 bound to Sepharose.™

Hepsin knock-out mice (U.S. Pat. No. 5,981,830), two females and one male, were immunized using the RIMMS protocol with pBacSurf Hepsin-gp64 expressed protein. Mice were initially immunized with 10 μg Hepsin/mouse in RIBI adjuvant, then boosted four times with 5 μg Hepsin/mouse according to the following schedule:

pBacSurf Hepsin-gp-64 Fusion Protein Immunization Schedule:

Day 1: Pre-Bleed, number mice and perform the 1^{st} immunization 10 μg/mouse RIMMS (150 μl pBACSurf-Hepsin-GP64, +450 μl RIBI 2×, +450 μl NaCl.)

Day 4: 1^{st} boost, 5 μg/mouse, RIMMS (75 μl BacSurf-Hepsin-GP64 Fusion Protein, +500 μl RIBI 2×, +500 μl NaCl.)

Day 6: 2^{nd} boost, 5 μg/mouse, RIMMS (75 μl BacSurf-Hepsin-GP64 Fusion Protein, +500 μl RIBI 2×, +500 μl NaCl.)

Day 8: 3^{rd} boost, 5 μg/mouse, RIMMS (75 μl BacSurf-Hepsin-GP64 Fusion Protein, +500 μl RIBI 2×, +500 μl NaCl.)

Day 11: 4^{th} boost, 5 μg/mouse, RIMMS, (75 μl BacSurf-Hepsin-GP64 Fusion Protein, +500 μl RIBI 2×, +500 μl NaCl.) Tail vein bleed and ELISA titer

ELISA Assay: Mouse Polyclonal Serum Titer

Two Dynatech Immulon II ELISA plates were coated with 100 ng/well (100 μl/well) Hepsin-ED-EK protein. Coating buffer, 50 mM Na₂CO₃ pH 9.6, was used to coat the wells with Hepsin-ED-EK protein in either ‘Native’ (without boiling in 1% SDS for 5 minutes at 90° C.) or ‘Denatured’ forms (with boiling in 1% SDS for 5 minutes at 90° C.) The plates were incubated overnight at 4° C. The wells were washed 3× with PBS 0.1% Tween-20, and blocked with 1 mg/ml BSA in PBS 0.1% Tween-20 for 30 minutes at room temperature. Added diluted mouse serum from 1:100 to 1:500,000 (dilutions done in 1 mg/ml BSA in PBS 0.1% Tween-20) in a 96-well tissue culture plate. The serum was incubated for one hour at 37° C., wash 3× with PBS 0.1% Tween-20. Goat-anti-mouse IgG (gamma chain specific at 1:10,000 in 1 mg/ml BSA in PBS 0.1% Tween-20) was added to the wells and incubated for 30 minutes at 37° C. (Sigma cat#A3673, lot #018H916). Each well was washed with 3× with PBS 0.1% Tween-20, developed with 100 μl Pierce TMB Kit for 3 minutes and stopped with 100 μl 1M H₂SO₄.

The Hepsin-ED-EK protein immunized mice had serum polyclonal antibodies that recognize Hepsin in the non-reduced and non-denatured form on Western Blot. The polyclonal antibodies did not recognize SDS denatured Hepsin-ED-EK protein on Western Blots.

Titer Levels

Protein Female #1: titer >1:500,000 serum dilution.

Protein Female #2: titer >1:500,000 serum dilution.

Protein Male: titer >1:100,000 serum dilution.

None of the BacSurf immunized mice had visible serum antibody titers. The BacSurf immunized mice did not recognize the denatured or native forms of Hepsin by Western Blot or ELISA (native only) analysis. This result was expected as reported in the BacSurf RIMMS paper.

Mouse Anti-Human-Hepsin MAbs

PEG Fusion Methods for Producing Hybridomas

The protein immunized mice Females #1 & 2 have polyclonal serum antibodies that are ELISA, FACS, and Western Blot Positive against native non-denatured Hepsin-ED-EK protein. The male protein immunized mouse has polyclonal serum antibodies that are ELISA and Western Blot positive against both native and denatured Hepsin-ED-EK protein. See above for data and notes on the immunizations.

Mouse myeloma cells (P3x63Ag8.653 mouse myeloma, LN2 ID#20164) were thawed into 10 ml warm IMDM+10% FBS in a 15 ml tube and spun for 8 minutes at 800 rpm. The supernatant was removed and the cells seeded in 20 ml IMDM+10% FBS in one T-75 flask.

The P3x63Ag8.653 cells were passaged according to standard methods. Briefly, the passages included the following:

Passage #2: cells in T-75 flask is 80% confluent and 95% viable. Seed the cells into one T-150 flask 1:3 to 60 ml, using the IMDM+1% FBS with HT media, to about 3.5% FBS concentration. Reseed T-75 to 20 ml with IMDM+1% FBS with HT media.

Passage #3-7: cells in T-150 flask is 90% confluent and 95% viable. Split the cells to T-150 flasks 1:3 to 60 ml each, using the IMDM+1% FBS with HT media, to about 1% FBS concentration.

Media Components:

100× 2-Mercapotethanol (2-ME):

100 ml DI H₂O Gibco catalog#15230-162,

1000× Transferrin (1 ml of 10 mg/ml)

9 ml IMDM salts (GIBCO CAT#12440-053; Sterile Filtered; 1000×=1 mg/ml)

IMDM+10% FBS:

IMDM 450 ml GIBCO CAT#12440-053

FBS 50 ml HYCLONE AHL9123 not heat inactivated.

L-GLU 5 ml 200 mM Gibco

1000× Transferrin 0.5 ml

2-ME 5 ML

Kanamycin 5 ML SIGMA K-0129 LOT#511K2381

Sterile filter 0.22 μm and store at 4° C.

IMDM+1% FBS:

550 ml IMDM salts and 5.5 ml FBS, and add all the above components using the same volumes as above.

And add HT 1VIAL SIGMA H-0137

Sterile filter 0.22 μm and store at 4° C.

Serum Free Media:

400 ml IMDM+4 ml Kanamycin and 4 ml 2-Mercaptoethanol, same components as above.

Red Blood Cell Lysing Solution (Sigma Catalog #R-7757)

Polyethylene Glycol (PEG) Solution:

Melted at 65° C. and add 5 ml of IMDM salts, sterile filtered, and keep at 65° C. until ready for fusion. Polyethylene Glycol (PEG): Sigma Catalog #P-2906

The P3xAg8.653 myeloma cells were prepared for PEG fusion using standard methods. Briefly, 300 ml of the myeloma cells were pooled from 4 T-150 flasks. The cells were counted, spun at 800 RPM for 8 minutes at room temperature, washed in IMDM Serum Free Media, and washed two more times. The myeloma cells were resuspended in a final volume of 25 ml of IMDM Serum Free Media.

The splenocytes were prepared according to standard methods. Briefly, the mice were killed by CO2 asphyziation and cervical dislocation. The blood was harvested via cardiac puncture, clotted, and spun at 10,000 RPM for 10 minutes. Approximately 1 ml of whole blood was harvested from each mouse. The lymph nodes and spleen were harvested. The spleenocytes and lymphocytes were harvested into 10 ml of IMDM Serum Free Media. The spleenocytes and lymphocytes were separated from the large particulate. The spleenocytes and lymphocytes were spun at 800 RPM for 8 minutes. The supernatant was aspirated and tossed. The red blood cells contained in the spleeenocyte/lymphocyte cell pellet were lysed with Red Blood Cell Lysing buffer with an under lay with 1 ml FBS, performed within 1 minute. Then Spun at 800 RPM for 8 minutes, removed the supernatant and FBS, resuspended the pellet in 10 ml IMDM Serum Free Media, counted the cells, and spun at 800 RPM for 8 minutes.

For the PEG fusion procedure, the myeloma and splenocytes were combined at a ratio of 1 myeloma cell: 5 splenocyte cells, in a 50 ml conical YES, and filled to 50 ml with IMDM Serum Free Media. The cells were spun at 800 RPM for 8 minutes, the pellet was warmed to 37 degrees in a water bath for 2 minutes then loosen the pellet. The PEG cell fusion step was performed in a 37 degree water bath. One ml 50% PEG was added over one minute, and mixed gently. The cells were spun at 400 RPM at room temperature for 2 minutes. To the cells were added 4.5 ml IMDM, 20% FBS complete media over 3 min. (without Aminopterin {A}). To the cells were added 5 ml IMDM, 20% FBS complete media (without Aminopterin {A}) over 2 min. The cells were spun at 800 RPM for 5 minutes at room temperature. The supernatant was removed. The pellet was resuspended in 35 ml IMDM, 20% FBS Complete Media (with HT and without Aminopterin {A}. The cells were allowed to anneal for 30 minutes at 37 degrees C. with gentle inversion and mixing.

Plating of Cells:

Fusion #1: The fused cells (Protein Female #1) were diluted to a plating volume of 650 ml in IMDM+20% FBS Complete Media with HT and plated at 125 micro liters per well. The fused cells were plated. A total of approximately 1.25×10⁸ cells were used for Fusion #1 and approximately 2.4×10⁴ cells per well.

Fusion #2: The fused cells (Protein Male) were diluted to a plating volume of 286 ml in IMDM+20% FBS Complete Media with HT and plated at 125 micro liters per well. The fused cells were plated. A total of approximately 5.5×10⁷ cells were used for Fusion #2 and approximately 2.4×10⁴ cells per well.

Electrofusion of Myeloma and Splenocytes

For the electrofusion procedure, the BTX Electro Cell Manipulator ECM 2001 was used. The BTX protocol 0116 was modified to use Mannitol instead of glucose. Combine the cells (ratio: 1 P3x63Ag8.653 to 5 spleenocytes) in one 50 ml conical and fill to 50 ml with IMDM Serum Free Media. Spin at 800 rpm for 8 minutes. Resuspend in 1 ml Fusion Buffer (0.3M Mannitol, 0.1 mM Ca⁺⁺ and 0.1 mM Mg⁺⁺ pH 7.0).

Electrofusion settings: Alignment Amplitude: 29 volts; Time: 10 seconds; Field Strength: 90V/cm; Electroporation Amplitude: 640 volts; Pulse Width: 30 micro seconds; Electrond: BTX Microslide P/N 453 (3.2 mm gap); Field Strength: 2 kV/cm. The optimal amplitude was determined by monitoring the alignment under an inverted microscope while adjusting the amplitude on the ECM 2001. In hood, sterilize the BTX Microslide P/N 453 (3.2 mm gap) by immersion in 95% ETOH with cover then dry in the hood by evaporation. Sterilize the Micrograbber cable by dipping in 95% ETOH. Using a sterile Pasteur Pipette, pipette the cell solution between the Microslide electrodes, 1 ml maximum. Place the sterile cover on the Microslide. Attach the Micrograbber cables to the posts of the microslide. Place the Microslide with cover in a newly opened and emptied sterile plastic bag. Close the bag by folding and taping the open side and allow the Micrograbber cables to extend out of the bag. Place the inverted microscope next to the BTX Electro Cell Manipulator ECM 2001. Bring the Microslide in the sterile and tapped shut bag to the inverted microscope. Tape the cables to the stage to prevent accidental movement of the Microslide. Connect the Micrograber cable to the BTX Electro Cell Manipulator ECM 2001. Press the automatic start button on the BTX Electro Cell Manipulator ECM 2001. The alignment of the cells can be monitored using the inverted microscope. Monitor the AC pulse on the BTX Enhancer 400 Graphic Pulse Display, and print out a copy of the display. After electrofusion carefully remove the Microslide from the sterile bag, in the hood. Using a sterile Pasteur Pipette, carefully aspirate the cells and place them in 35 ml of IMDM+20% FBS Complete Hybridoma platting media with HT. Wash the space between the electrodes two times with Fusion Buffer, to remove any remaining cells, using a sterile Pasteur Pipette. Keep these cells and add them to the cells previously harvested. The Microslide is now ready for the next fusion. Repeat steps the previous steps until all cells have been fused. Let cells anneal for a minimum of 30 minutes at 37° C. in 35 ml of IMDM+20% FBS Complete Hybridoma platting media with HT. Invert gently every 6 minutes.

Electrofusions:

Fusion #3

Electrofusion of the Protein Immunized Female Mouse #1 cells was performed in two batches, the first fusion with 1.0 ml of cells and the second with 0.5 ml of cells. All cells were combined. The cells were plated at a high density, approximately 5.4×10⁴ cells per well. Dilute Fusion #3 (Protein Female #1, Electrofusion) to plating volume of 287.5 ml in IMDM+20% FBS Complete Media with HT and plate at 125 micro liters per well. The cells were plated in 23 plates.

Fusion #4

Electrofusion of the Protein Immunized Male Mouse cells was done in one batch, with 1.0 ml of cells. The cells were plated at a high density, at approximately 2.6×10⁴ cells per well. Dilute Fusion #4 (Protein Male, Electrofusion) to plating volume of 262.5 ml in IMDM+20% FBS Complete Media with HT and plated at 125 micro liters per well. The cells were plated in 21 plates.

### Example 6

The following provides a description of the methods used for performing a Western blot analysis of the antibodies described in Example 5 above.

Polyclonal Antibodies

A 2-well prep gel 12% tri-glycine (Invitrogen, #EC6009) was used for the Western Blot. Prep gel sample solution was prepared using 4.5 micro gram protein: 1 micro liter modified hepsin zymogen, 124 micro liter water, 125 micro liter 2× sample buffer (Invitrogen, #LC2676) with 20% beta-Mercaptoethanol. Sample solution was heated to 100 degrees C. for 5 minutes then cooled on ice for 3 minutes. 250 micro liters of sample solution was loaded onto the gel. The gel ran at 200 V for 1 hour in tris-glycine running buffer (Invitrogen, #LC2675). The proteins were transferred i.e., blotted, onto PVDF membrane using seven volts overnight in a Novex transfer apparatus. The membrane was blocked for 4 days in phosphate buffer saline with 0.1% Tween (PBST) and 5% powdered milk. Polyclonal serum, 1:500 dilution, was incubated with the membrane for two hours at room temperature. The membrane was washed twice with PBST, ten minutes for each wash. At the end of the second wash the secondary antibody (Pierce, Cat#31444, anti mouse IgG/IgM, 1:5000) was added and incubated for one hour at room temperature. The membrane was washed as previously described then incubated in 5 ml of Amersham-ECL plus solution for 1 min. The membrane was covered with plastic wrap and exposed to film (Kodak Bio-Max MR) for one and five minutes then developed.

Monoclonal Antibodies

A 2-well prep gel 12% tri-glycine (Invitrogen, #EC6009) was used for the Western Blot. Prep gel sample solution was prepared using 4.5 micro gram protein: 1 micro liter modified hepsin zymogen (4.5 μg/μl concentration), 124 micro liter water, 125 micro liter 2× sample buffer (Invitrogen, #LC2676) with 20% beta-Mercaptoethanol. Sample solution was heated to 100 degrees C. for 5 minutes then cooled on ice for 3 minutes. 250 micro liters of sample solution was loaded onto the gel. The gel ran at 200 V for 1 hour in tris-glycine running buffer (Invitrogen, #LC2675). The proteins were transferred to PVDF membrane using seven volts overnight in a Novex transfer apparatus. The Blot was blocked for 4 days in phosphate buffer saline with 0.1% Tween (PBST) and 5% powdered milk. 600 micro liters of supernatant i.e., the hybridoma condition medium, was added to each slot, one slot for each hybridoma. The supernatant was incubated with the membrane for two hours at room temperature. The membrane was washed twice with PBST, ten minutes for each wash. At the end of the second wash the secondary antibody (Pierce, Cat#31444, anti mouse IgG/IgM, 1:5000) was added and incubated for one hour at room temperature. The membrane was washed as previously described then incubated in 5 ml of Amersham-ECL plus solution for 1 min. The membrane was covered with plastic wrap and exposed to film (Kodak Bio-Max MR) for one and five minutes then developed.

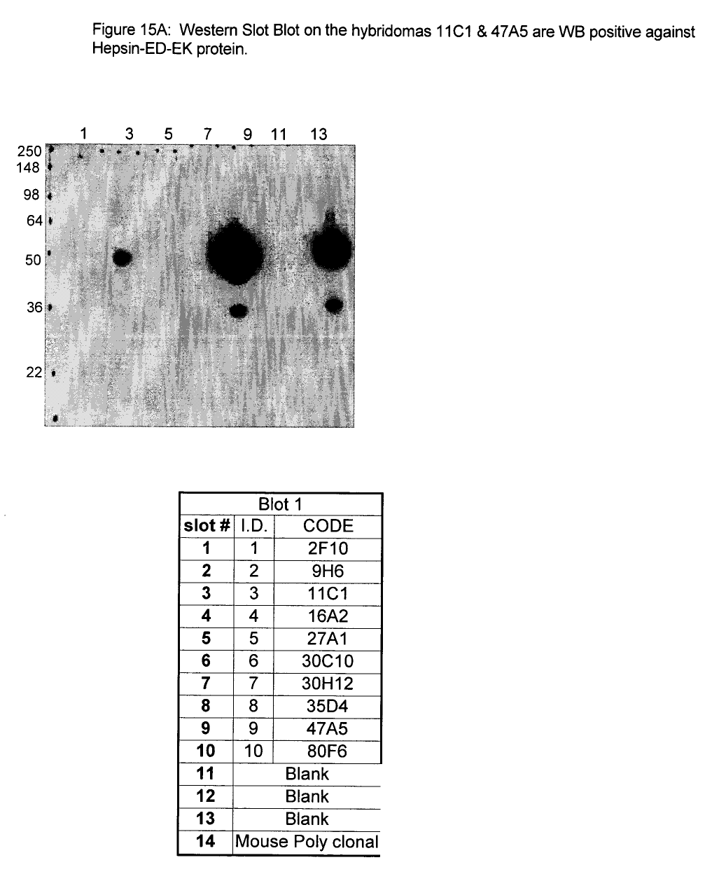

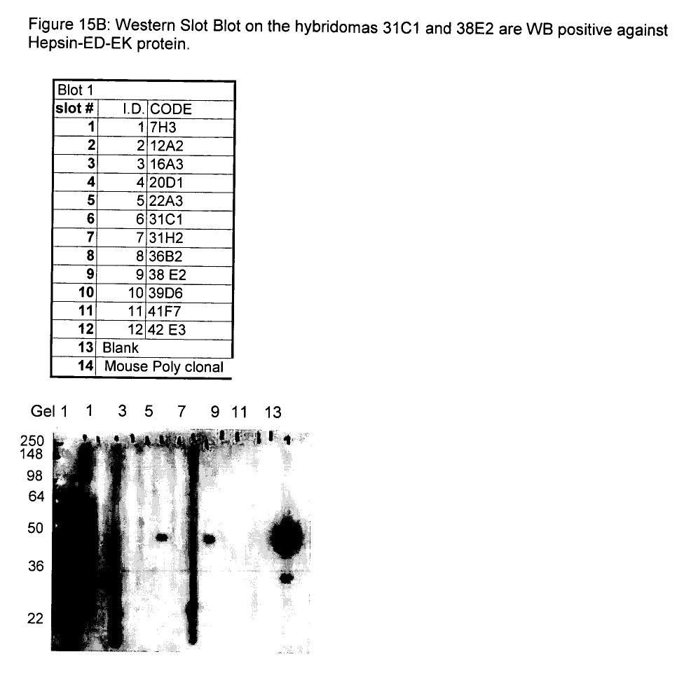

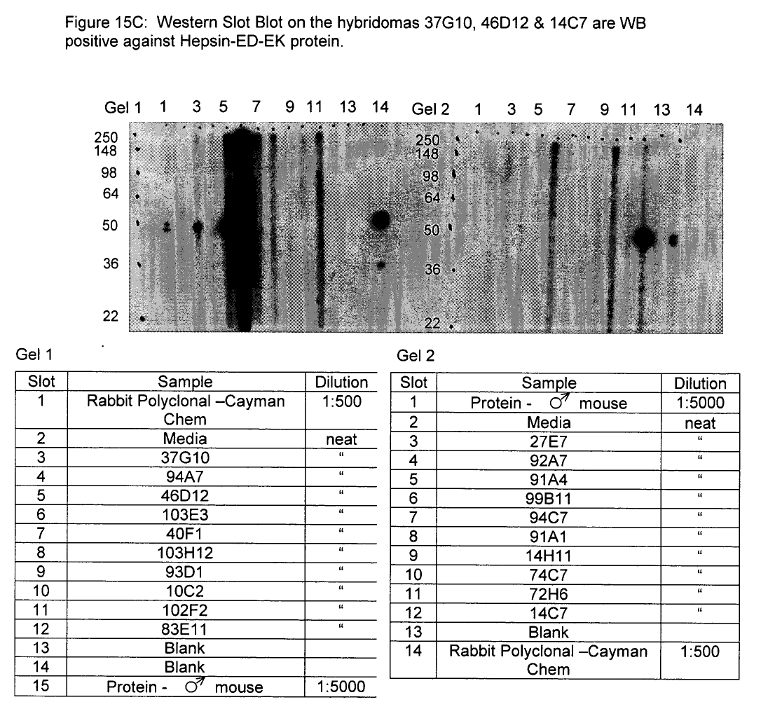

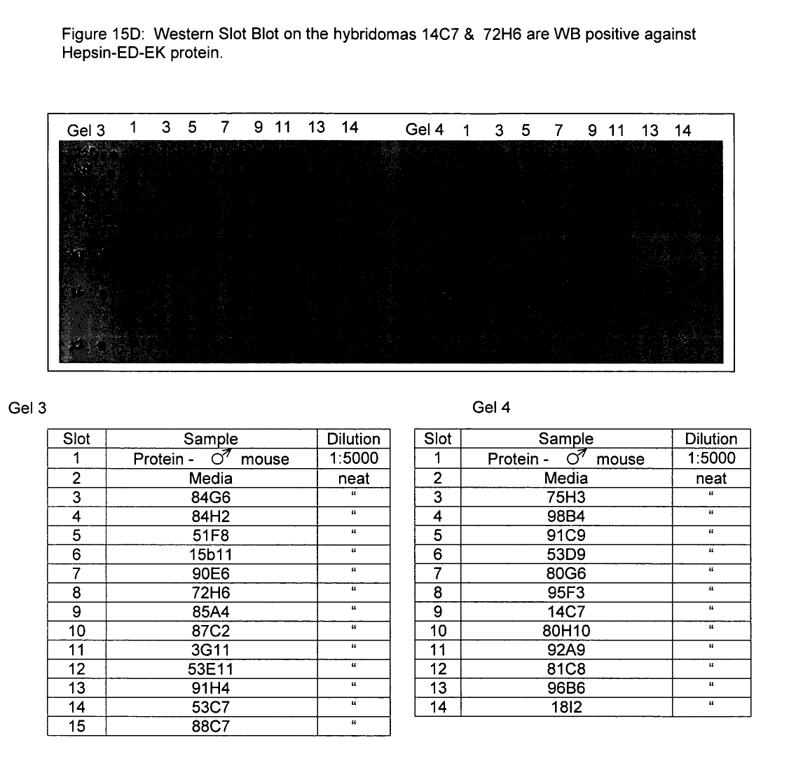

The following eight monoclonal antibodies bind to hepsin: 47A5, 14C7, 46D12, 38E2, 37G0, 31C1, 11C1 and 72H6 (FIGS. 15A-D).

### Example 7

The following provides a description of the Biacore methods used for performing the kinetic analyses, used to determine binding affinities and kinetic constants (KD, ka and kd) for the antibodies described in Example 5 above.

Rabbit polyclonal antibodies, specific for mouse Fc, were covalently attached to the CM5 Sensor Chip via primary amine coupling and used to immobilize mouse anti-hepsin monoclonal antibodies. Several concentrations of purified hepsin were then passed over the surface to enable binding to the immobilized antibody. Hepsin binding was correlated with increases in surface plasmon resonance (SPR) by the instrument. Release of the hepsin-antibody complex was similarly measured by passing buffer over the surface and measuring the decrease in SPR.

The rabbit anti-mouse Fc surface was prepared using standard primary amine coupling methods and antibody concentration of 100 ug/ml. The Protein G-purified antibodies described in Example 5 above (e.g., 94A7) were diluted to 200 nM in HEPES buffered saline, HBS-EP (Biacore, BR-1001-88). This buffer was also used as the mobile phase throughout the assay. The purified hepsin protein used was expressed in 293 cells and activated with enterokinase. Four concentrations of hepsin were run in the assay: 45, 22.5, 11.25, 5.625 and 0 nM. The low rate used in the assay was 10 ul per minute. The chip was regenerated between each cycle using 10 mM glycine, pH 1.8 to remove the captured antibody and antibody-bound hepsin, leaving the immobilized anti-mouse antibody ready for the subsequent cycle.

Using this method and the BiaEvaluation 3.0 software provided with the instrument, both the association rate constant (kₐ), the dissociation rate constant (k_{d}) and the equilibrium constant (K_{D}) were calculated using a 1:1 Langmuir model to fit the association and dissociation phases. Rate constants for two monoclonal antibodies: 1A12 and 94A7 are shown below.

| Antibody | Kₐ (1/Ms) | k_{d} (1/s) | KD (M) | Chi2 |
|---|---|---|---|---|
| 1A12 (IgG1k) | 1.90 × 10⁵ | 3.49 × 10⁻⁴ | 1.84 × 10⁻⁹ | 9.31 |
| 94A7 (IgG2ak) | 5.35 × 10⁵ | 9.1 × 10⁻⁵ | 2.31 × 10⁻¹⁰ | 1.95 |

### Example 8

The following provides a description of the methods used for performing an assay for neutralization activity of the antibodies described in Example 5 above.

Antibody neutralization was tested by preincubating the purified antibodies with hepsin expressing BHK cells, (i.e., control cells), and then assaying hepsin activity on the cells using factor VII.

Typical assay conditions were as follows:

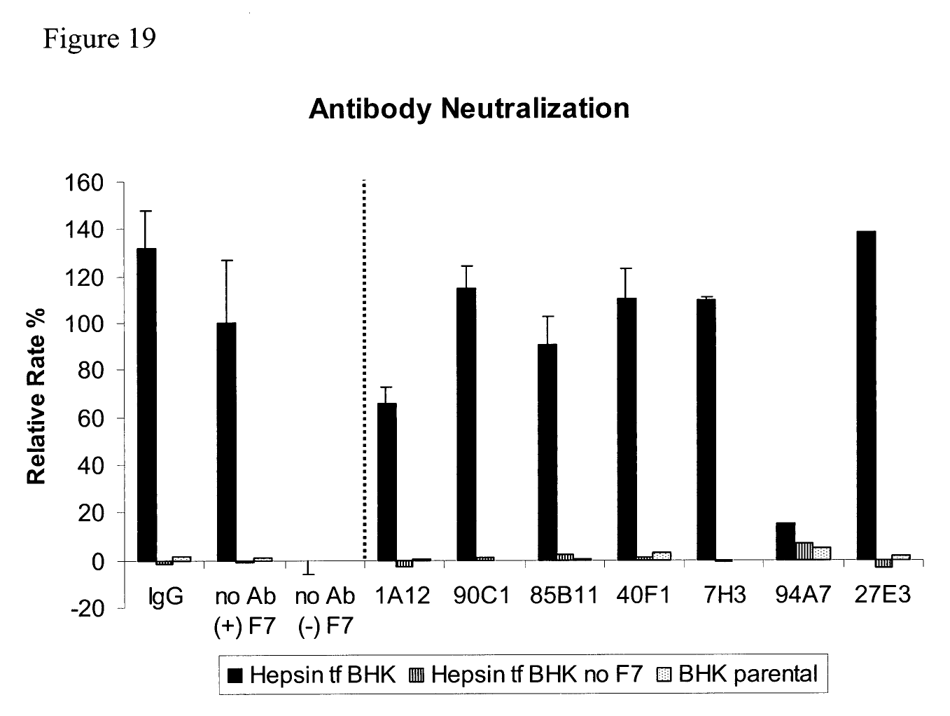

1.75 million cells were incubated with factor VII (10 ug) in Tris buffer pH 7.4 (50 mM), NaCl (150 mM), CaCl₂ (2.5 mM), PEG 6000 (0.1%), at 37° C. for 20 mins. Thereafter the suspension was centrifuged to remove the cells and the supernatant assayed for VIIa activity. This was carried out in using chromogenic substrate S-2266 (Chromogenix) (1.6 mM) in the same buffer. Enzyme activity was followed by measuring O.D.405 over a 15 min time period (See, for example, FIG. 19).

### Example 9

The following provides a description of the immunohistochemistry methods used for detecting expression of naturally-occurring hepsin molecule in tissue samples from prostate cancer patients.

Tissue samples from prostate cancer patients were embedded in paraffin, sliced and placed on microscope slides. The slides were bathed in Xylene 3 times, and in 100% and 95% ethyl alcohol, 3 times each for 2 minutes, and washed in PBS. The slides were incubated in Peroxo-Block (Zymed Lab) for 1 minute, and washed in PBS. The slides were incubated in Protein blocking solution (Dako) for 10-15 minutes. The slides were incubated in the following antibodies or control solutions overnight at room temperature.

Polyclonal Antibody Staining

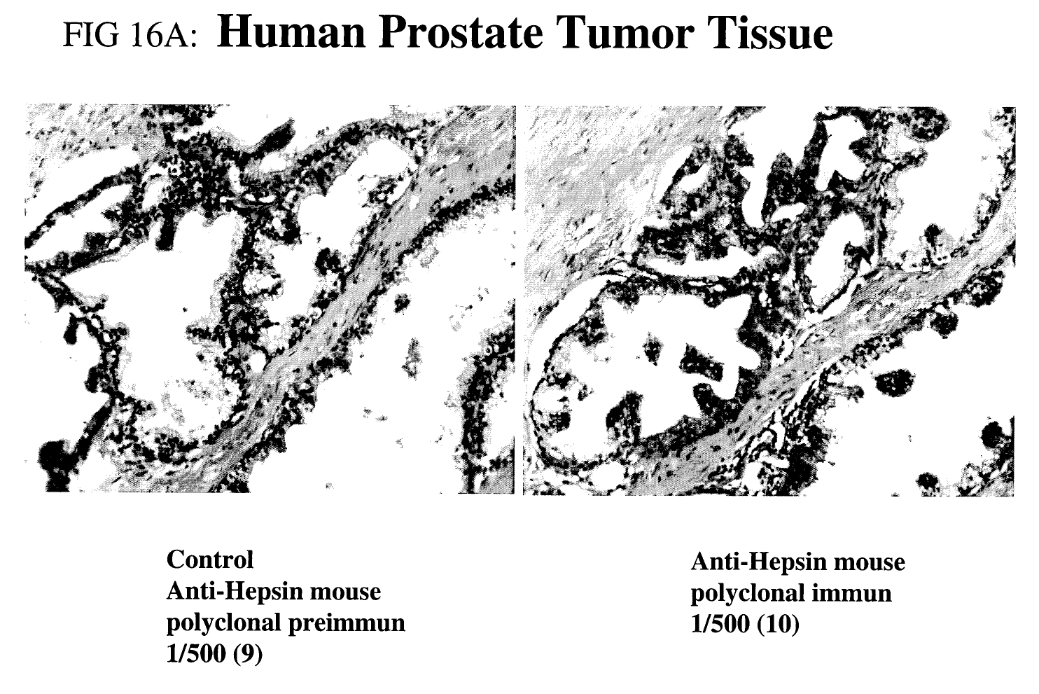

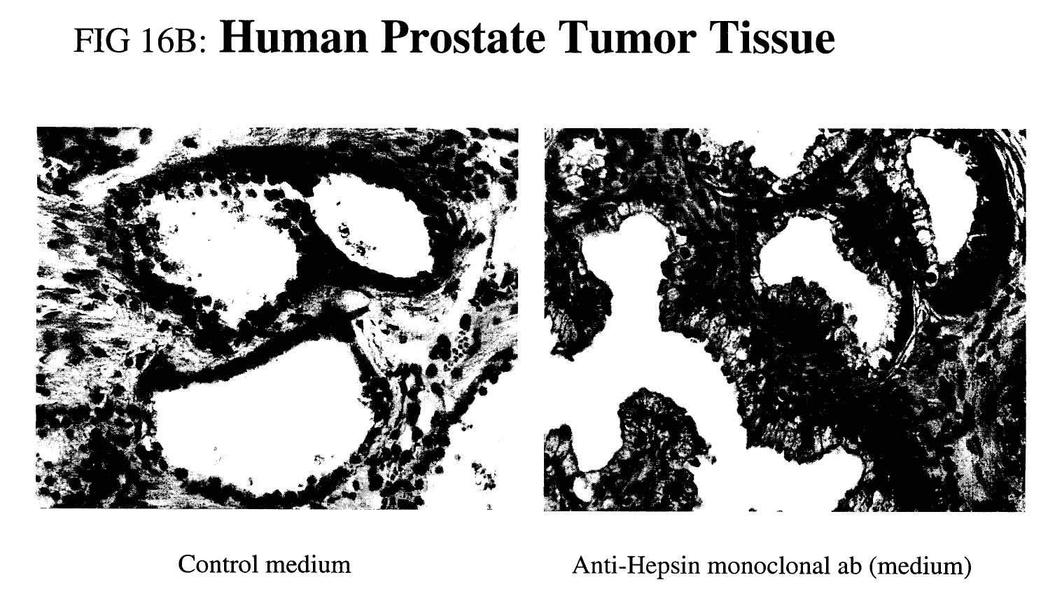

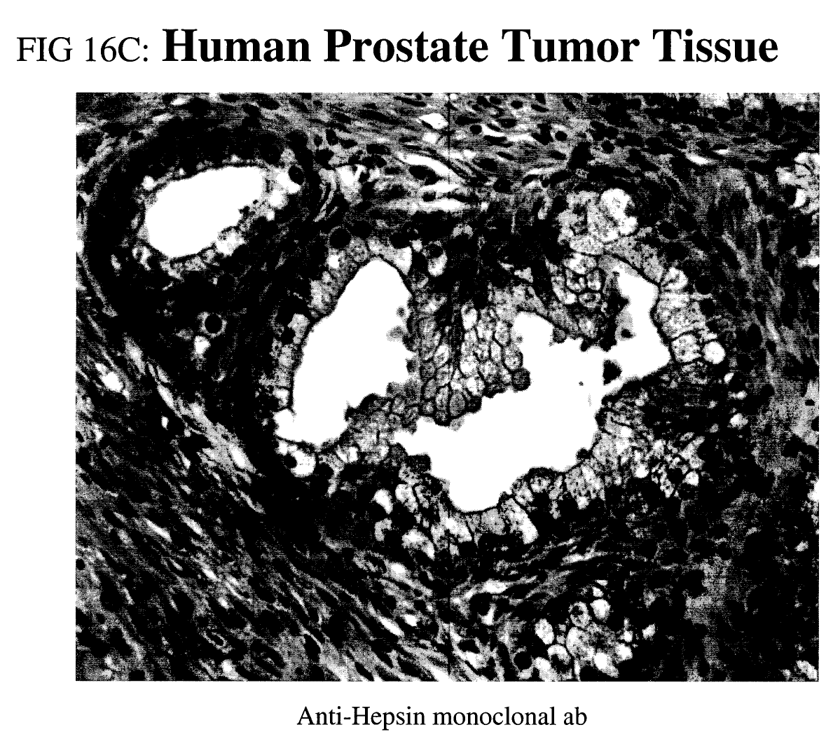

Mouse #10 polyclonal anti-hepsin antibody immune-serum, at 1/500 dilution, was used to stain human prostate tumor tissue (FIG. 16A: left panel). Pre-immune-serum i.e., serum taken from a mouse before immunization, for mouse #9 was used as negative control to stain human prostate tumor tissue (FIG. 16A: right panel).

Monoclonal Antibody Staining

Mouse monoclonal anti-hepsin antibody culture supernatant, i.e. the supernatant from hybridoma cell lines that contain anti-hepsin monoclonal antibody 11C1, was used to stain human prostate tumor tissue (FIG. 16B; right panel; FIG. 16C). Cell culture media was used as a negative control to stain human prostate tumor tissue (FIG. 16B; left panel).

The slides were washed in PBS, and incubated with anti-mouse biotinylated secondary antibody at 2 ug/ml for 45 minutes. The slides were washed in PBS, and incubated in Strepavidin conjugated horse-radish peroxidase (2 ug/ml) for 40 minutes at room temperature. The slides were washed in PBS.

The slides were developed using NeoRed Substrate kit (Vector Laboratories, Inc) for 8-10 minutes. The slides were washed in PBS. The slides were counterstained using QS hemotoxylin for 1 minute, and washed in water.

The slides were dehydrated in 95%, 100% ethanol and Xylene 3 times each for 1 minute. The slides were viewed under the microscope.

### Example 10

The following provides a description of methods for detecting the presence of cells expressing cell surface hepsin molecule, using fluorescence-activated cell sorting (FACS).

The FACS Analysis can use one T150 and one T75 Flask of 80% confluent HEPG2 cells. HEPG2 is a human hepatoma derived cell line which was used originally by Leytus to clone the hepsin gene (Leytus et al., 1988, Biochemistry. 27 (3):1067-74; Knowles et al., 1980, Human hepatocellular carcinoma cell lines secrete the major plasma proteins and hepatitis B surface antigen. Science 209:497-499; Fair and Bahnak, 1984, Human hepatoma cells secrete single chain factor X, prothrombin, and antithrombin III. Blood 64:194-204; Darlington et al., 1987, Growth and hepatospecific gene expression of human hepatoma cell in a defined medium. In Vitro Cell Dev Biol. 23:349-354)

The cells are rinsed with phosphate buffered saline (PBS, without Mg²⁺ and Ca²⁺). Twenty to thirty ml of Cell Dissociation Buffer, (Invitrogen, #13151-014) is added to the flasks to release the cells from the flask surface. The detached cells are centrifuged into a pellet and resuspended in 15 ml ice-cold PBS. The cells are divided into aliquots of 6.1×10⁶ cells/ml and are centrifuged into a pellet. The cells are resuspended in a primary antibody (see below) at 1:100 in 100 micro liter PBS. The cells are incubated on ice for 1 hour and washed twice with PBS.

The cells are incubated with a secondary antibody (1:100 Flourescein-anti-mouse IgG H+L in PBS, Vector Laboratories, Inc., #FI-2000), incubated for 30 minutes on ice, and washed twice with PBS (1 ml per wash) after incubation. The cells are resuspended in 350 micro liters PBS, transferred to a FACS tube, and 5 micro liters of propidium Iodine is added to each sample. Each sample is then read on a FACS machine.

The primary antibodies (e.g., polyclonal, mouse anti-serum) includes: pre-bleed male mouse; HEPG2-immunized male mouse; pre-bleed female mouse #1; HEPG2-immunized female mouse #1; pre-bleed female mouse #2; and HEPG2-immunized female mouse #2.

### Example 11

The following provides a description of the methods used for performing a chromogenic and fluorogenic, substrate-based assay for detecting the presence of activated, modified hepsin protease and/or for identifying a compound of interest from a library of candidate compounds, where the compound of interest inhibits the activity of the modified hepsin protease.

Detecting Activated, Modified Hepsin

The modified hepsin molecule is purified using an affinity column with anti-V5 bound to Sepharose.™

Activation of the V5-purified modified hepsin molecule includes 2 μM modified hepsin molecule, 3 units/ml enterokinase (EKMax, Invitrogen) in a 5 mM Tris, 25 mM NaCl, EKMax buffer at about pH 8.08. Hepsin activity is check at various time points. The enterokinase can be removed or remain in the activation mixture after the hepsin is activated.

The presence of activated modified hepsin protease is detected in an assay, including 1 nM or 5 nM of the modified hepsin protease, and 200 μM substrate in 100 mM Hepes, 100 mM NaCl pH 7.4 at room temperature, and is monitored at 405 nm on Molecular Devices SpectraMax 250.

The assay is performed in 384-well plates at pH 7.4. The modified activated hepsin protease (e.g., activated by the enterokinase reaction described in Example 4, supra) is reacted with the chromogenic substrate Val-Leu-Arg-pNA (Chromogenix, cat. # S-2266). In this assay, the modified hepsin protease liberates the para-nitro-analine (pNA), resulting in absorbance at 405 nm. The assay buffer includes 100 mM HEPES pH 7.4, 100 mM NaCl. The concentration of the modified hepsin protease is 250 pM, the substrate concentration was 40 uM, and the candidate compounds are screened at 2 uM. The assay is performed for 90 minutes and the reaction is terminated by adding 5 micro liters of 0.15N HCl. The absorbance at 405 nm is read using a Wallac “Victor V”.

The chromogenic substrates are obtained from Chromogenix. The kinetic constants for the various substrates are shown in Table 2.

Detecting Hepsin Inhibitors

The screening assay for identifying compounds exhibiting inhibitory activity of the modified hepsin protease is performed by screening molecules for hepsin-inhibitory activity.

Approximately 460,000 molecules from the Berlex compound library are tested in the hepsin inhibitor screen.

Out of the initial unconfirmed hits, fewer compounds are confirmed upon retest. Unconfirmed hits are defined as any compound that inhibited hepsin activity by at least 60% in terms of the average between the x and y locations of that particular compound (compounds appear twice in their respective library pools). The maximum allowed delta between the x and y results is 20%. Confirmed hits are defined as having an IC50 value<=10 uM, and a hill slope<2.0.

What is claimed is:

1. An isolated, modified hepsin molecule, or fragment or derivative thereof, comprising a substitute activation sequence.

2. The isolated molecule of claim 1, wherein the substitute activation sequence replaces a wildtype activation sequence RIVGG of FIG. 17.

3. The isolated molecule of claim 1, wherein the substitute activation sequence is DDDDKIVGG as shown in FIG. 18.

4. The isolated molecule of claim 1, having the amino acid sequence as shown in FIG. 18.

5. The isolated molecule of claim 1, wherein the substitute activation sequence is any one of SEQ ID NOS: 1-4.

6. The isolated molecule of claim 1, wherein the substitute activation sequence is recognized and cleaved by a protease.

7. The isolated molecule of claim 1, wherein the substitute activation sequence is recognized and cleaved by a serine protease.

8. The isolated molecule of claim 1, wherein the substitute activation sequence is recognized and cleaved by a type II transmembrane protease.

9. The isolated molecule of claim 1, wherein the substitute activation sequence is DDDDK-IVGG (SEQ ID NO.: 3), which is recognized and cleaved by enterokinase.

10. The isolated molecule of claim 1, wherein the substitute activation sequence is recognized and cleaved by thrombin, clotting factor Xa, furin, trypsin, chymotrypsin, elastase, thrombin, plasmin, kallikrein, aerosin, human airway trypsin-like protease (HAT), mast cell tryptase, MBL-associated serine proteases (MASP-1 and MASP-2), corin, MT-SP1/matryptase, TMPRSS2 or Stubble-stubbloid

11. The isolated molecule of claim 1, further comprising a signal peptide sequence.

12. The isolated molecule of claim 11, wherein the signal peptide sequence is bacterial, fungal, insect, plant, or animal.

13. The isolated molecule of claim 11, wherein the signal peptide is an Igκ signal sequence.

14. The isolated molecule of claim 1, further comprising an epitope tag.

15. The isolated molecule of claim 14, wherein the epitope tag is an amino acid tag.

16. The isolated molecule of claim 14, wherein the epitope tag is histidine or cysteine.

17. The isolated molecule of claim 14, wherein the epitope tag is V5 or flag.

18. The isolated molecule of claim 1, which is from a prokaryote or eukaryote source.

19. The isolated molecule of claim 18, wherein the eukaryote is a mammal.

20. The isolated molecule of claim 19, wherein the mammal is bovine, porcine, murine, equine, canine, feline, avian, piscine, ovine, insects, simian, or human animal.

21. An activated modified hepsin molecule, comprising a substitute activation sequence cleaved by a protease.

22. A method for detecting hepsin cleavage activity in a sample, comprising contacting the functionally-active hepsin molecule of claim 21 with a substrate under conditions so that the functionally active hepsin molecule cleaves the substrate and detecting the substrate cleavage products thereby indicating hepsin cleavage activity.

23. The method of claim 22, wherein the substrate is a chromogenic or fluorogenic substrate.

24. The method of claim 22, wherein the substrate is N-benzoyl-Leu-Ser-Arg-pNA.HCl, N-benzoyl-Ile-Glu-Phe-Ser-Arg-pNA.HCl, or N-benzoyl-Phe-Val-Arg-pNA.HCl.

25. The isolated, modified hepsin molecule of claim 1, wherein the substitute activation sequence has been cleaved thereby producing a modified activated hepsin molecule.

26. An isolated nucleic acid molecule encoding the modified hepsin molecule of claims 1 or 25.

27. A complementary nucleic acid molecule, comprising a nucleotide sequence complementary to the nucleic acid molecule of claim 26.

28. The nucleic acid molecule of claim 25 which is DNA or RNA.

29. The nucleic acid molecule of claim 26 which is a peptide nucleic acid molecule (PNA).

30. The nucleic acid molecule of claim 26 which is a phosphorothioate derivative molecule.

31. The nucleic acid molecule of claim 26 which is labeled so as to directly or indirectly produce a detectable signal with a compound selected from the group consisting of a radiolabel, an enzyme, a chromophore and a fluorescer.

32. A vector comprising the nucleic acid molecule of claim 26.

33. The vector of claim 32, wherein the vector is a plasmid, cosmid, BAC, YAC, PAC or a phagemid.

34. A host vector system comprising the vector of claim 32 in a suitable host cell.

35. The host vector system of claim 34, wherein the suitable host cell is a prokaryotic or eukaryotic cell.

36. The host vector system of claim 35, wherein the prokaryotic cell is a bacterial cell.

37. The host vector system of claim 35, wherein the eukaryotic cell is a yeast, plant, insect or mammalian cell.

38. The host vector system of claim 37, wherein the insect cell is Sf21.

39. A DNA sequence as depicted in FIG. 9, FIG. 10, or FIG. 11.

40. A method for detecting in a sample the presence of a nucleic acid molecule encoding a modified hepsin molecule, comprising contacting the sample with the nucleic acid molecule of claim 26 and detecting a complex formed between the nucleic acid molecule and a constituent in the sample or between the complementary nucleic acid molecule and a constituent in the sample, wherein the complex indicates the presence of the nucleic acid molecule encoding a modified hepsin molecule in the sample.

41. The method of claim 40, wherein the constituent is an RNA or cDNA molecule.

42. The method of claim 40, wherein the sample is a tissue, a cell, or a biological fluid.

43. The method of claim 42, wherein the biological fluid is urine, blood sera or phlegm.

44. The method of claim 42, wherein the sample is from prostate, liver, kidney, pancreas, stomach, thyroid, testes, or ovary.

45. A method for inducing an immune response in a subject, comprising administering the modified hepsin molecule of claim 1 to the subject.

46. A method for producing an antibody, comprising administering the modified hepsin molecule of claim 1 to a subject.

47. The method of claim 46 wherein the subject is a hepsin knock-out mouse.

48. An antibody, or fragment or derivative thereof, which binds a modified hepsin molecule.

49. An Fab, F(ab′)₂ or Fv fragment of the antibody of claim 48.

50. The antibody of claim 48 which is a polyclonal antibody or monoclonal antibody.

51. A recombinant protein comprising the antigen-binding region of the antibody of claim 48.

52. An antibody which competes for binding to the same epitope as the epitope bound by the antibody of claim 48.

53. The antibody of claim 48 which is a chimeric antibody.

54. The antibody of claim 53, wherein the chimeric antibody comprises a human region and a murine region.

55. The antibody of claim 48 which is a humanized antibody.

56. The antibody of claim 48 which is a neutralizing antibody.

57. An idiotypic antibody of the modified hepsin molecule of claim 1.

58. An immunoconjugate comprising the antibody of claim 48 joined to a therapeutic agent.

59. The immunoconjugate of claim 58, wherein the therapeutic agent is a cytotoxic agent.

60. The immunoconjugate of claim 59, wherein the cytotoxic agent is selected from the group consisting of ricin, doxorubicin, daunorubicin, taxol, ethiduim bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, glucocorticoid and radioisotopes.

61. A hybridoma which produces the antibody of claim 48.

62. A hybridoma deposited with the American Type Culture Collection and designated ATCC PTA-4561.

63. A hybridoma deposited with the American Type Culture Collection and designated ATCC ______.

64. A monoclonal antibody produced by the hybridoma of claim 61.

65. A pharmaceutical composition, comprising the antibody of claim 48 and a suitable carrier.

66. A pharmaceutical composition, comprising the molecule of claim 1 and a suitable carrier.

67. The pharmaceutical composition of claim 65 or 66, wherein the suitable carrier is selected from a group consisting of a phosphate buffered saline solution, water, emulsions, oil/water emulsion, wetting agents, sterile solutions, excipients, starch, milk, sugar, clay, gelatin, stearic acid, salts of stearic acid, magnesium stearate, calcium stearate, talc, vegetable fats or oils, gums, and glycols.

68. The pharmaceutical composition of claim 65 or 66 which is formulated as a liposome, polymeric composition, or polymer microsphere.

69. The pharmaceutical composition of claim 65 or 66 which is formulated as a tablet, coated tablet, or capsule.

70. A method for binding a hepsin molecule, comprising contacting a sample with the antibody of claim 48 so as to bind the hepsin molecule.

71. A method for detecting a hepsin molecule, comprising contacting a sample with the antibody of claim 48 and detecting the binding of the antibody with the hepsin molecule in the sample.

72. The method of claim 71, wherein the detecting comprises determining whether a complex is formed between the hepsin molecule and the antibody, wherein the complex indicates the presence of the hepsin molecule in the sample.

73. A method for detecting the presence of hepsin molecule in a subject, comprising administering to the subject the antibody of claim 48, and detecting the binding of the hepsin molecule with the antibody with the hepsin molecule in the subject.

74. The method of claim 73, wherein the detecting comprises determining whether a complex is formed between the hepsin molecule and the antibody, wherein the complex indicates the presence of the hepsin molecule in the subject.

75. A method for diagnosing a cancer expressing hepsin in a subject, comprising quantitatively determining in a sample from the subject the amount of a hepsin molecule using the antibody of claim 48, and comparing the amount of the hepsin molecule in a sample from a normal subject, the presence of a measurably different amount of the hepsin molecule between the sample from the subject and the sample from the normal subject indicating the presence of a cancer expressing hepsin in the subject.

76. A method for measuring the prognosis of a cancer expressing hepsin molecule in a subject, comprising quantitatively determining in a sample from the subject the amount of a hepsin molecule using the antibody of claim 48, and comparing the amount of the hepsin molecule in a sample from a normal subject, the presence of a measurably different amount of the hepsin molecule between the sample from the subject and the sample from the normal subject indicating the prognosis of the cancer expressing hepsin in the subject.

77. A method for monitoring the course of a cancer expressing hepsin molecule in a subject, comprising quantitatively determining in a first sample from the subject the amount of a hepsin molecule using the antibody of claim 48, and comparing the amount so determined with the amount of hepsin molecule present in a second sample from the subject, wherein the first and second samples are obtained from the subject at different points in time, a difference in the amounts of hepsin molecule in the first and second sample being indicative of the course of the cancer expressing hepsin molecule in the subject.

78. A method for inhibiting growth of a cell expressing hepsin molecule, comprising contacting the cell with the antibody of claim 48, so as to inhibit growth of the cell.

79. A method for killing a cell expressing hepsin, comprising contacting the cell with the antibody of claim 48 so as to kill the cell.

80. A method for inhibiting metastasis of a cancer cell expressing hepsin, comprising contacting the cancer cell with the antibody of claim 48.

81. A method for inhibiting angiogenesis of a cancer cell expressing hepsin, comprising contacting the cell with the antibody of claim 48.

82. The method of claim 78 or 79, wherein the cell is from a prostate, prostate cancer, metastasis of prostate cancer, liver, liver cancer, metastasis of liver cancer, kidney, kidney cancer, metastasis of kidney cancer, pancreas, pancreatic cancer, metastasis of pancreatic cancer, stomach, stomach cancer, metastasis of stomach cancer, thyroid, thyroid cancer, metastasis of thyroid cancer, testes, testicular cancer, metastasis of testicular cancer, ovary, ovarian cancer, or metastasis of ovarian cancer.

83. A method for producing an antibody that recognizes endogenous hepsin, comprising administering a modified hepsin molecule to a subject and producing the antibody.

84. A vaccine comprising the molecule of claim 1.

85. A kit comprising the nucleic acid molecule of claim 26.
